(19) United States
(12) Patent Application Publication
(10) Pub. No.: US 2007/0202528 A1
(43) Pub. Date: Aug. 30, 2007

(54) Methods for modulating proteins not previously known as proteases

(76) Inventors: Anthony G. Day, San Francisco, CA (US); David A. Estell, San Mateo, CA (US); Eric H. Lyons, El Cerrito, CA (US); Jian Yao, Sunnyvale, CA (US)

Correspondence Address: GENENCOR INTERNATIONAL, INC.;ATTENTION: LEGAL DEPARTMENT, 925 PAGE MILL ROAD, PALO ALTO, CA, 94304, US

(21) Appl. No.: 11/707,223

(22) Filed: Feb. 15, 2007

Related U.S. Application Data
(63) Continuation of application No. 10/618,281, filed on Jul. 11, 2003, now abandoned.
(60) Provisional application No. 60/395,325, filed on Jul. 12, 2002.

(51) Int. Cl. C12Q 1/68; G01N 33/574; C12P 21/06; C12N 9/64; C07K 16/40

(52) U.S. Cl. 435/6; 435/7.23; 435/68.1; 435/226; 530/388.26

(57) ABSTRACT

The present invention relates to the proteins not previously identified as proteases; the use of those peptides in screening for compounds that modulate protease activity; treating individuals in need of treatment with the compounds or proteases; and in methods for diagnosing a disease or disorder associated with a protease of the instant invention.

### RELATED APPLICATIONS

The present application claims priority to U.S. Provisional Patent Application Ser. No. 60/395,325, entitled “Methods for Modulating Proteins Not Previously Known as Proteases” (Attorney Docket No. GC773P) filed Jul. 12, 2002 by Day et al.

### FIELD OF THE INVENTION

The present invention relates to enzymes which, hitherto, have not been used to hydrolyze peptide bonds and have not been identified as having proteolytic activity, and their novel use as proteases and to the identification of compounds that modulate their protease activity. The invention also relates to the use of the novel proteases and identified compounds to treat individuals having a disease or disorder involving a protease-mediated pathway.

### BACKGROUND

Proteases are enzymes that breakdown peptide bonds by irreversibly catalyzing the hydrolysis of bond(s) in substrates. They are generally classified as either exopeptidases that cleave amino acids from the ends of a protein, or as endopeptidases, which cleave peptide bonds within the protein. Some recognize specific sequences and cleave proteins only once or twice, while others degrade proteins completely into amino acids. Some proteases are secreted to cause the destruction of proteins in extracellular material while others are secreted into an. area, such as the stomach, to breakdown proteins, such as those present in foods. Others are involved in regulating physiological processes via biological cascades, and may be expressed intracellularly or extracellularly and may be soluble membrane anchored or integral membrane proteins.

Proteolytic mechanisms are involved in a large number of diverse processes within the body. Their normal functions include modulation of apoptosis (caspases) (Salvesen and Dixon, Cell, 1997, 91:443-46), control of blood pressure (renin, angiotensin-converting enzymes) (van Hooft et al., 1991, N Engl J Med. 324 (19):1305-11, and chapters 254 and 359 in Barrett et al., HANDBOOK OF PROTEOLYTIC ENZYMES, 1998, Academic Press, San Diego), tissue remodeling and tumor invasion (collagenase) (Vu et al., 1998, Cell 93:411-22, Werb, 1997, Cell, 91:439-442), development of Alzheimer's Disease (.beta.-secretase) (De Strooper et al., 1999, Nature 398:518-22), protein turnover and cell-cycle regulation (proteosome) (Bastians et al., 1999, Mol. Biol. Cell. 10:3927-41, Gottesman, et al., 1997, Cell, 91:435-38, Larsen et al., 1997, Cell, 91:431-34), inflammation (TNF-.alpha. convertase) (Black et al., Nature, 1997, 385:729-33), and protein turnover (Bochtler et al., 1999, Annu. Rev. Biophys Biomol Struct. 28:295-317). Proteases may be classified into several major groups including serine proteases, cysteine proteases, aspartyl proteases, metalloproteases, threonine proteases, and other proteases.

### 1. Aspartyl Proteases

Aspartyl proteases, also known as acid proteases, are a widely distributed family of proteolytic enzymes in vertebrates, fungi, plants, retroviruses and some plant viruses. Aspartate proteases of eukaryotes are monomeric enzymes which consist of two domains. Each domain contains an active site centered on a catalytic aspartyl residue. The two domains most probably evolved from the duplication of an ancestral gene encoding a primordial domain. Enzymes in this class include cathepsin E, renin, presenilin (PS 1), and the APP secretases.

### 2. Cysteine Proteases

Another class of proteases which perform a wide variety of functions within the body are the cysteine proteases. Among their roles are the processing of precursor proteins, and intracelluar degradation of proteins marked for disposal via the ubiquitin pathway. Eukaryotic cysteine proteases are a family of proteolytic enzymes which contain an active site cysteine. Catalysis proceeds through a thioester intermediate and is facilitated by a nearby histidine side chain; an asparagine completes the essential catalytic triad. Peptidases in this family with important roles in disease include the caspases, calpain, hedgehog, and Ubiquitin hydolases.

Cysteine proteases are produced by a large number of cells including those of the immune system (macrophages, monocytes, etc.). These immune cells exercise their protective role in the body, in part, by migrating to sites of inflammation and secreting molecules, among the secreted molecules are cysteine proteases.

Under some conditions, the inappropriate regulation of cysteine proteases of the immune system can lead to autoimmune diseases such as rheumatoid arthritis. For example, the over-secretion of the cysteine protease cathepsin C causes the degradation of elastin, collagen, laminin, and other structural proteins found in bones. Bone subjected to this inappropriate digestion is more susceptible to metastasis.

Caspase—Apopotosis

A cascade of protease reactions is believed to be responsible for the apoptotic changes observed in mammalian cells undergoing programmed cell death. This cascade involves many members of the aspartate-specific cysteine proteases of the caspase family, including caspases 2, 3, 6, 7, 8 and 10 (Salvesen and Dixit, Cell 1997, 91:443-446). Cancer cells that escape apoptotic signals, generated by cytotoxic chemotherapeutics or loss of normal cellular survival signals (as in metastatic cells), can go on to develop palpable tumors.

Calpain—Axonal Death, Dystrophies

Calcium-dependent cysteine proteases, collectively called calpain, are widely distributed in mammalian cells (Wang, 2000, Trends Neurosci. 23(1):20-26). The calpains are nonlysosomal intracellular cysteine proteases. The mammalian calpains include 2 ubiquitous proteins, CAPN1 and CAPN2, as well as 2 stomach-specific proteins, and CAPN3, which is muscle-specific (Herasse et al., 1999, Mol. Cell. Biol. 19 (6):4047-55). The ubiquitous enzymes consist of heterodimers with distinct large subunits associated with a common small subunit, all of which are encoded by different genes. The large subunits of calpains can be subdivided into 4 domains; domains I and III, whose functions remain unknown, show no homology with known proteins. The former, however, may be important for the regulation of the proteolytic activity. Domain II shows similarity with other cysteine proteases, which share histidine, cysteine, and asparagine residues at their active sites. Domain IV is calmodulin-like. CAPN5 and CAPN6 differ from previously identified vertebrate calpains in that they lack a calmodulin-like domain IV (Ohno et al., 1990, Cytogenet. Cell Genet. 53 (4):225-29).

Hedgehog—Cancer

The organization and morphology of the developing embryo are established through a series of inductive interactions. One family of vertebrate genes has been described related to the Drosophila gene ‘hedgehog’ (hh) that encodes inductive signals during embryogenesis (Johnson and Tabin, 1997, Cell 90:979-990). “Hedgehog” encodes a secreted protein that is involved in establishing cell fates at several points during Drosophila development (Marigo et al., 1995, Genomics 28:44-51). There are three known mammalian homologs of hh: Sonic hedgehog (Shh), Indian hedgehog (Ihh), and desert hedgehog (Dhh) (Johnson and Tabin, 1997, Cell 90:979-990). Like its Drosophila cognate, Shh encodes a signal that is instrumental in patterning the early embryo. It is expressed in Hensen's node, the floorplate of the neural tube, the early gut endoderm, the posterior of the limb buds, and throughout the notochord (Chiang et al., 1996, Nature 383:407-413). It has been implicated as the key inductive signal in patterning of the ventral neural tube, the anterior-posterior limb axis, and the ventral somites. Oro et al., Science 276: 81 7-821, 1997, showed that transgenic mice overexpressing SHH in the skin developed many features of the basal cell nevus syndrome, demonstrating that SHH is sufficient to induce basal cell carcinomas (BCCs) in mice. The data suggested that SHH may have a role in human tumorigenesis. Activating mutations of SHH or another ‘hedgehog’ gene may be an alternative pathway for BCC formation in humans. The human mutation his133tyr (his134tyr in mouse) is a candidate. It is distinct from loss-of-function mutations reported for individuals with holoprosencephaly (Oro et al., 1997, Science 276:817-821). His133 lies adjacent in the catalytic site to his134, one of the conserved residues thought to be necessary for catalysis. SHH may be a dominant oncogene in multiple human tumors, a mirror of the tumor suppressor activity of the opposing ‘patched’ (PTCH) gene (Aszterbaum et al., 1998, J. Invest. Derm. 110:885-888). The rapid and frequent appearance of Shh-induced tumors in the mice suggested that disruption of the SHH-PTC pathway is sufficient to create BCCs.

Ubiquitin Hydrolases—Apoptosis, Checkpoint Integrity

Ubiquitin carboxyl-terminal hydrolases (3.1.2.15) (deubiquitinating enzymes) are thiol proteases that recognize and hydrolyze the peptide bond at the C-terminal glycine of ubiquitin. These enzymes are involved in the processing of poly-ubiquitin precursors as well as that of ubiquinated proteins. In eukaryotic cells, the covalent attachment of ubiquitin to proteins plays a role in a variety of cellular processes. In many cases, ubiquitination leads to protein degradation by the 26S proteasome. Protein ubiquitination is reversible, and the removal of ubiquitin is catalyzed by deubiquitinating enzymes, or DUBs. A defect in these enzymes, catalyzing the removal of ubiquitin from ubiquinated proteins, may be characteristic of neurodegenerative diseases such as Alzheimer's, Parkinson's, progressive supranuclear palsy, and Pick's and Kuf's disease. Papain—Cathepsins K S and B, are also useful for bone resorbtion, and Ag processing (Prosite PS00139).

Cysteine Protease AEP

The cysteine protease AEP plays another role in the immune functions. It has been implicated in the protease step required for antigen processing in B cells. Manoury et al. Nature 396:695-699 (1998).

### 3. Metalloproteases

Collagenase—Invasion

Matrix degradation is an essential step in the spread of cancer. The 72- and 92-kD type IV collagenases are members of a group of secreted zinc metalloproteases which, in mammals, degrade the collagens of the extracellular matrix. Other members of this group include interstitial collagenase and stromelysin (Nagase et al., 1992, Matrix Suppl. 1:421-424). By targeted disruption in embryonic stem cells, Vu et al. (Cell, 1998, 934:11-22) created homozygous mice with a null mutation in the MMP9/gelatinase B gene. These mice exhibited an abnormal pattern of skeletal growth plate vascularization and ossification. Growth plates from MMP9-null mice in culture showed a delayed release of an angiogenic activator, establishing a role for this proteinase in controlling angiogenesis.

MMP2 (gelatinase A) have been associated with the aggressiveness of human cancers (Chenard et al., 1999, Int. J. Cancer, 82:208-12). In a study comparing basal cell carcinomas (BCC) with the more aggressive squamous cell carcinomas (SCC), both MMP2 and MMP9 were expressed at a higher level in SCC (Dumas et al., 1999, Anticancer Res., 19 (4B):2929-38). Additionally, expression of MMP2 and MMP9 in T lymphocytes has recently been shown to be modulated by the Ras/MAP kinase signaling pathways (Esparza et al., 1999, Blood, 94:2754-66) (see also, Li et al., 1998, Biochim. Biophys. Acta, 1405:110-20).

ADAMS—TNF, Inflammation Growth Factor Processing

The ADAM peptidases are a family of proteins containing a disintegrin and metalloproteinase (ADAM) domain (Werb and Yan, Science, 1998, 282:1279-1280). Members of this family are cell surface proteins with a unique structure possessing both potential adhesion and protease domains (Primakoff and Myles, Trends in Genet., 2000, 16:83-87). Activity of these proteases can be linked to TNF, inflammation, and/or growth factor processing.

ADAM proteases have also been characterized as having a pro- and metalloproteinase domain, a disintegrin domain, a cysteine-rich region and an EGF repeat (Blobel, 1 997, Cell, 90:589-592 which is hereby incorporated herein by reference in its entirety including any figures, tables, or drawings). They have been associated with the release from the plasma membrane of numerous proteins including Tumor Necrosis Factor-.alpha. (TNF-.alpha.), kit-ligand, TGF.alpha., Fas-ligand, cytokine receptors such as the II-6 receptor and the NGF receptor, as well as adhesion proteins such as L-selectin, and the b amyloid precursor proteins. (Blobel, 1997, Cell, 90:589-592).

Tumor necrosis factor-.alpha. is synthesized as a proinflammatory cytokine from a 233-amino acid precursor. Conversion of the membrane-bound precursor to a secreted mature protein is mediated by a protease termed TNF-.alpha. convertase. TNF-.alpha. is involved in a variety of diseases. ADAM17, which contains a disintegrin and metalloproteinase domains, is also called ‘tumor necrosis factor-.alpha. converting enzyme’ (TACE) (Black et al., Nature, 1997, 385:729-33). The gene encodes an 824-amino acid polypeptide containing the features of the ADAM family: a secretory signal sequence, a disintegrin domain, and a metalloprotease domain. Expression studies showed that the encoded protein cleaves precursor tumor necrosis factor-.alpha. to its mature form. This enzyme may also play a role in the processing of Transforming Growth Factor-.alpha. (TGF-.alpha.), as mice which lack the gene are similar in phenotype to those that lack TGF-.alpha. (Peschon et al., Science, 282:1281-1284, 1998).

Neprylisin—Endothelin-Converting Enzyme

Carboxypeptidases specifically remove COOH-terminal basic amino acids (arginine or lysine). They have important functions in many biologic processes, including activation, inactivation, or modulation of peptide hormone activity, neurotransmitter processing, and alteration of physical properties of proteins and enzymes.

Dipeptidase—ACE

Angiotensin I converting enzyme (EC 3.4.15.1), or kininase II, is adipeptidyl carboxypeptidase that plays an important role in blood pressure regulation and electrolyte balance by hydrolyzing angiotensin I into angiotensin II, a potent vasopressor, and aldosterone-stimulating peptide. The enzyme is also able to inactivate bradykinin, a potent vasodilator. Although angiotensin-converting enzyme has been studied primarily in the context of its role in blood pressure regulation, this widely distributed enzyme has many other physiologic functions. There are two forms of ACE: a testis-specific isozyme and a somatic isozyme which has two active centers.

Matrix Metalloproteases—Tissue Remodeling And Inflammation

The matrix metalloproteases (MMPs) are a family of related matrix-degrading enzymes that are important in tissue remodeling and repair during development and inflammation (Belotti et al., 1999, Int. J. Biol. Markers 14(4):232-38). Abnormal expression is associated with various diseases such as tumor invasiveness (Johansson and Kahari, 2000, Histol. Histopathol. 15(l):225-37), arthritis (Malemud et al., 1999, Front. Biosci. 4:D762-71), and atherosclerosis (Nagase, 1997, Biol. Chem. 378 (3-4):151-60). MMP activity may also be related to tobacco-induced pulmonary emphysema (Dhami et al., Am. J. Respir. Cell Mol. Biol., 2000, 22:244-52).

Metalloprotease Processing of Growth Factors

In addition to the processing of TGF-.alpha. described above, metalloproteases have been directly demonstrated to be active in the processing of the precursor of other growth factors such as heparin-binding EGF (proHB-EFG) (Izumi et al., EMBO J, 1998, 17:7260-72), and amphiregulin (Brown et al., 1998, J. Biol. Chem., 27:17258-68).

Additionally, metalloproteases have recently been shown to be instrumental in the communication whereby stimulation of a GPCR pathway results in stimulation of the MAP kinase pathway (Prenzel et al., 1999, Nature, 402:884-888). The growth factor intermediate in the pathway, HB-EGF is released by the cell in a proteolytic step regulated by the GPCR pathway involving an uncharacterized metalloprotease. After release, the HB-EGF is bound by the extracellular matrix and then presented to the EGF receptors on the surface, resulting in the activation of the MAP kinase pathway (Prenzel et al., 1999, Nature, 402:884-888).

A recent study by Gallea-Robache et al., 1997, Cytokine, (5):340-6, has also implicated a metalloprotease family displaying different substrate specificites in the shedding of other growth factors including macrophage colony-stimulating factor (M-CSF) and stem cell factor (SCF) (Gallea-Robache et al., 1997, Cytokine 9:340-46). The shedding of M-CSF (also known as CSF-1) has been linked to activation of Protein Kinase C by phorbol esters (Stein et al., 1991, Oncogene, 6:601-05).

### 4. Serine Proteases

The serine proteases are a class which includes trypsin, kallikrein, chymotrypsin, elastase, thrombin, tissue plasminogen activator (tPA), urokinase plasminogen activator (uPA), plasmin (Werb, Cell, 1997, 91:439-442), kallikrein (Clements, Biol. Res., 1998, 31 (3): 151-59), and cathepsin G (Shamamian et al., Surgery, 2000, 127:142-47). These proteases have in common a well-conserved catalytic triad of amino acid residues in their active site consisting of histidine-57, aspartic acid-102, and serine-195 (using the chymotrypsin numbering system). Serine protease activity has been linked to coagulation and they may have use as tumor markers.

Serine proteases can be further subclassified by their specificity in substrates. The elastases prefer to cleave substrates adjacent to small aliphatic residues such as valine, chymases prefer to cleave near large aromatic hydrophobic residures, and tryptases prefer positively charged residues. One additional class of serine protease has been described recently which prefers to cleave adjacent to a proline. This prolyl endopeptidase has been implicated in the progression of memory loss in Alzheimer's patients (Toide et al., 1998, Rev. Neurosci. 9 (1):17-29).

A partial list of proteases known to belong to this large and important family include: blood coagulation factors VII, IX, X, XI and XII; thrombin; plasminogen; complement components C1r, C1s, C2; complement factors B, D and I; complement-activating component of RA-reactive factor; elastases 1, 2, 3A, 3B (protease E); hepatocyte growth factor activator; glandular (tissue) kallikreins including EGF-binding protein types A, B, and C; NGF-.gamma. chain, gamma.renin, and prostate specific antigen (PSA); plasma kallikrein; mast cell proteases; myeloblastin (proteinase 3) (Wegener's autoantigen); plasminogen activators (urokinase-type, and tissue-type); and the trypsins I, II, III, and IV. These peptidases play key roles in coagulation, tumorigenesis, control of blood pressure, release of growth factors, and other roles. (http://www.babraham.co.uk/Merops/Merops.htm).

### 5. Threonine Peptidases—(Prosite PDOC00326/PDOC00668) Proteasomal Subunits

The proteasome is a multicatalytic threonine proteinase complex involved in ATP/ubiquitin dependent non-lysosomal proteolysis of cellular substrates. It is responsible for selective elimination of proteins with aberrant structures, as well as naturally occurring short-lived proteins related to metabolic regulation and cell-cycle progression (Momand et al., 2000, Gene 242 (1-2):15-29, Bochtler et al., 1999, Annu. Rev. Biophys Biomol Struct. 28:295-317). The proteasome inhibitor lactacystin reversibly inhibits proliferation of human endothelial cells, suggesting a role for proteasomes in angiogenesis (Kumeda, et al., Anticancer Res. 1999 September-October; 19 (5B):3961-8). Another important function of the proteasome in higher vertebrates is to generate the peptides presented on MHC-class 1 molecules to circulating lymphocytes (Castelli et al., 1 997, Int. J. Clin. Lab. Res. 27 (2):103-10). The proteasome has a sedimentation coefficient of 26S and is composed of a 20S catalytic core and a 22S regulatory complex. Eukaryotic 20S proteasomes have a molecular mass of 700 to 800 kD and consist of a set of over 15 kinds of polypeptides of 21 to 32 kD. All eukaryotic 20S proteasome subunits can be classified grossly into 2 subfamilies, alpha. and beta., by their high similarity with either the alpha. or beta. subunits of the archaebacterium Thermoplasma acidophilum (Mayr et al., 1999, Biol. Chem. 380 (10):1183-92). Several of the components have been identified as threonine peptidases, suggesting that this class of peptidases plays a key role in regulating metabolic pathways and cell-cycle progression, among other functions (Yorgin et al., 2000, J. Immunol 164 (6):2915-23).

### 6. Peptidases of Unknown Catalytic Mechanism

The prenyl-protein specific protease responsible for post-translational processing of the Ras proto-oncogene and other prenylated proteins falls into this class. This class also includes several viral peptidases that may play a role in mammalian infection, including cardiovirus endopeptidase 2A (encephalomyocarditis virus) (Molla et al., 1993, J. Virol 67 (8):4688-95), NS2-3 protease (hepatitis C virus) (Blight et al., 1998, Antivir. Ther. 3 (Suppl 3):71-81), endopeptidase (infectious pancreatic necrosis virus) (Lejal et al., J. Gen. Virol., 2000, 81:983-992), and the Npro endopeptidase (hog cholera virus) (Tratschin et al., 1998, J. Virol. 72 (9):7681-84).

Consequently, proteases, as well as protease agonists and antagonists, are useful as therapeutic agents in treating various conditions or diseases and in diagnostic and research practices.

Proteases are also of commercial and industrial importance, as they are used to process leather and wool, produce food and beverages and to manufacture of cleaning products.

### SUMMARY

The present disclosure identifies the proteins having SEQ ID NOs 1-92 as proteases where the sequences had not been so identified. As a result, the present invention is directed to a method of identifying a test or endogenous compound that modulates the protease activity of a protein selected from the group consisting of SEQ ID NOs. 1-92, or a functional variant thereof, comprising (i) combining (a) a protease comprising a sequence of any one of SEQ ID NOs. 1-92, or a functional variant or fragment thereof, (b) a compound and (c) a substrate for said protein and (ii) detecting an alteration in the interactions between the protease and the substrate in the presence and absence of the test compound.

Thus the present invention provides proteases described in any one of SEQ ID NOs. 1-92. See “List 1” below. The present invention also provides nucleic acid sequences encoding proteins described in any one of SEQ ID NOs. 1-92.

Thus, the present invention contemplates a method of cleaving a peptide bond in a desired protein comprising contacting said desired protein with a protease comprising a sequence selected from the group consisting of SEQ ID NOs. 1-92, under conditions wherein the protease hydrolyzes at least one peptide bond in the desired protein.

Another embodiment is to a method for identifying a compound that modulates the activity of a protease comprising, (a) contacting a protease having an amino acid sequence selected from the group consisting SEQ ID NOs. 1-92 or a functional fragment or variant thereof, with a test compound; (b) measuring the activity of said protease before and after said contacting step; and (c) determining whether said test compound modulates the activity of said protease.

In one embodiment, the method further comprises contacting a substrate for the protease before and after contacting the protease with the test compound. In another embodiment, the detecting step comprises measuring the level of proteolytic activity. In another embodiment, this detecting step comprises measuring the amount of product generated from cleavage of the substrate by the protease. In yet another embodiment, the test compound is an inhibitor of proteolytic function of the protease. In another embodiment, the test compound is a competitive inhibitor. In one other embodiment, the test compound is an activator of proteolytic function of the protease.

The present invention also contemplates a method for identifying a compound that modulates the activity of a protease in a cell comprising (a) expressing, in a cell, a protease having an amino acid sequence selected from the group consisting SEQ ID NOs 1-92; (b) exposing said cell to a test compound; and (c) monitoring an alteration in cell phenotype or proteolytic activity.

In another embodiment, the invention envisions method for treating a disease or disorder by administering to a patient in need of such treatment a compound that modulates the activity of a protease having an amino acid sequence selected from the group consisting of SEQ ID NOs 1-92. In one embodiment, the compound modulates protease activity in vitro. In another embodiment, the compound is a protease inhibitor.

In yet another aspect of the present invention, there is provided a method for detection of a protease in a sample as a diagnostic tool for a disease or disorder, comprising (a) contacting the sample with a nucleic acid probe which hybridizes under hybridization assay conditions to a nucleic acid target encoding a protease having an amino acid sequence selected from the group consisting of SEQ ID NOs 1-92, or fragments thereof, or the complements of the sequences and fragments thereof; and (b) detecting the presence or amount of the probe:target region hybrid as an indication of the disease.

In another aspect, a method for detection of a protease in a sample as a diagnostic tool for a disease or disorder is provided. This method comprises (a) comparing a nucleic acid target region encoding a protease in a sample, wherein the protease has an amino acid sequence selected from the group consisting of SEQ ID NOs 1-92 or one or more fragments thereof, with a control nucleic acid target region encoding the protease polypeptide, or one or more fragments thereof; and (b) detecting differences in nucleotide or predicted amino acid sequence or amount between the target region and the control target region, as an indication of said disease or disorder.

Another method of the present invention is for treating a disease or disorder by administering to a patient in need of such treatment a pharmaceutical composition comprising a compound that modulates the activity of a protease having an amino acid sequence selected from the group consisting of SEQ ID NOs 1-92.

In another aspect, a method for treating a disease or disorder is provided, wherein the method comprises administering to a patient in need of such treatment a pharmaceutical composition comprising a protease having an amino acid sequence selected from the group consisting of SEQ ID NOs 1-92.

In either method, the pharmaceutical composition further comprises an excipient selected from the group consisting of calcium carbonate, calcium phosphate, various sugars, starches, cellulose derivatives, gelatin, and polymers such as polyethylene glycols.

Also provided by the present invention is an antibody that binds to a part of a protein comprising the sequence described in any one of SEQ ID NOs. 1-92. In another embodiment, the antibody is used to identify and/or detect the presence of protease polypeptides in a sample. In another embodiment, the antibody is used to monitor cell cycle regulation or to determine immuno-localization of protease polypeptides within a cell. In another embodiment, the antibody is therapeutically effective.

The present invention also contemplates a method of treating an individual in need of treatment, comprising administering to the individual a protein comprising a sequence described in any one of SEQ ID NOs. 1-92, or a functional variant thereof. In one embodiment, the administering step is achieved by injecting, swallowing, infusing, topically applying or inhaling an aerosol. In another embodiment, the protein may be in the form of a pharmaceutical composition.

In another embodiment, the individual is a mammal. In another embodiment, the mammal is selected from the group consisting of a human, primate, rat, mouse, rabbit, pig, cattle, sheep, goat, cat or dog. In another embodiment, the mammal is a human.

Yet another aspect of the invention envisions a method for identifying a compound that modulates the activity of a protease comprising, (a) contacting a protease having an amino acid sequence selected from the group consisting SEQ ID NOs 1-92, or a functional variant thereof with a test compound; (b) measuring the catalytic activity of the protease; and (c) determining whether the test compound modulates the activity of the protease and/or binds to the protease.

A further aspect entails a method for identifying a compound that modulates (e.g., inhibits or stimulates) the activity of a protease in a cell comprising (a) expressing, in a cell, a protease having an amino acid sequence, or a fragment thereof, selected from the group consisting SEQ ID NOs 1-92; (b) exposing the cell to a test compound; and (c) monitoring a change in cell phenotype or proteolytic activity. In one other aspect, the invention provides a method for treating a disease or disorder by administering to a patient in need of such treatment a compound that modulates the activity of a protease having an amino acid sequence selected from the group consisting of SEQ ID NOs 1-92. In one embodiment, the compound modulates protease activity in vitro. In another embodiment, the compound is a protease inhibitor.

The present invention may be used to treat diseases or disorders which involve, as an example without limitation, the following genes: GD2, Lewis-Y, 72 kd glycoprotein (gp72, decay-accelerating factor, CD55, DAF, C3/C5 convertases), CO17-1A (EPCAM, 17-1A, EGP-40), TAG-72, CSAg-P (CSAp), 45kd glycoprotein, HT-29 ag, NG2, A33 (43kd gp), 38kd gp, MUC-1, CEA, EGFR (HER1), HER2, HER3, HER4, HN-1 ligand, CA125, Syndecan-1, Lewis-X, PgP, FAP stromal Ag (fibroblast activation protein), EDG Receptors (endoglin receptors), ED-B, Laminin-5 (gamma2), Cox-2(+LN-5), AlphaVbeta3 integrin, AlphaVbeta5 integrin, uPAR (urokinase plasminogen activator receptor), Endoglin (CD105) and Folate receptor osteopontin. Others involved are well-known by those skilled in the art. Or, other diseases or disorders discloses herein or which are well-known in the art.

Thus, in another embodiment, the disease or disorder is selected from the group consisting of cancers, immune-related diseases and disorders, cardiovascular disease, brain or neuronal-associated diseases, and metabolic disorders. The disease or disorder is selected from the group consisting of cancers of tissues; cancers of hematopoietic origin; diseases of the central nervous system; diseases of the peripheral nervous system; Alzheimer 's disease; Parkinson 's disease; multiple sclerosis; amyotrophic lateral sclerosis; viral infections; infections caused by prions; infections caused by bacteria; infections caused by fungi; and ocular diseases.

In another embodiment, the disease or disorder is selected from the group consisting of migraines; pain; sexual dysfunction; mood disorders; attention disorders; cognition disorders; hypotension; hypertension; psychotic disorders; neurological disorders; dyskinesias; metabolic disorders; and organ transplant rejection.

One other aspect of the invention envisages a method for detecting a protease in a sample as a diagnostic tool or marker or biomarker for a disease or disorder, comprising (a) contacting the sample with a nucleic acid probe which hybridizes under hybridization assay conditions to a nucleic acid target encoding a protease having an amino acid sequence selected from the group consisting of SEQ ID NOs 1-92, or a functional variant thereof, or complements thereof; and (b) detecting the presence or amount of the probe:nucleic acid target hybrid as an indication of the disease.

### DETAILED DESCRIPTION OF THE PREFERRED EMBODIMENTS

The present invention uses proteins which, hitherto, have not been used to hydrolyze peptide bonds and have not been identified as having proteolytic activity, to screen for compounds that modulate protease activity and for treating individuals having a disease or disorder involving a pathway in which one or more protease are involved via the compound or protease, itself.

The inventors recognized that isolated proteins having sequences described in SEQ ID NOs. 1-92, or a functional variant thereof are capable of hydrolyzing peptide bonds because their primary amino acid structure comprises proteolytic domains, when previously not though to do so. Accordingly, the invention provides novel uses of proteins as protease enzymes. The term “protease” refers to a protein or polypeptide sequence represented by SEQ ID NOS: 1-92 and includes functional variants thereof, as well as fragments derived from the polypeptides and variants. Variants and fragments of the invention have protease activity. The full-length protein sequence, a variant or a fragment thereof, can be isolated or purified from a cell that naturally expresses it, or produced by recombinant, chemical, or known protein synthesis methods, as provided herein.

A polypeptide that retains “protease activity” is one that retains the ability to catalyze the hydrolysis of a peptide bond. The ninety-two proteins identified as proteases in the present invention, can be serine-, cysteine-, aspartic-, threonine-, or metallo-proteases, based upon the sequences of their active and catalytic domains. The “active domain” refers to the region of a protein having a sequence described in any one of SEQ ID NOs. 1-92, that contains amino acid residues that perform the catalytic function of the protease; see Table 2 below which lists the boundaries of the “active domains” for each of the ninety-two identified proteases of the present invention. Similarly, the “catalytic domain” refers to the amino acid residues in any one of the protein sequences of SEQ ID NOs. 1-92 that are integral in catalyzing a chemical reaction, such as in hydrolysis of peptide bonds. Thus, the term “catalytic activity” defines the rate at which a protease catalytic domain cleaves a substrate. The term “substrate” as used herein refers to a polypeptide or protein or other molecule known to one skilled in the art which is cleaved by a protease of the invention.

The term “cleaved” refers to the severing of a covalent bond between amino acid residues or other moieties.

The term “therapeutic effect” refers to the inhibition, activation or replacement of factors causing or contributing to the abnormal condition. A therapeutic effect relieves to some extent one or more of the symptoms of the abnormal condition. In reference to the treatment of abnormal conditions, a therapeutic effect can refer to, without limitation, one or more of the following: (a) an increase in the proliferation, growth, and/or differentiation of cells; (b) inhibition (i.e., slowing or stopping) of cell death; (c) inhibition of degeneration; (d) relieving to some extent one or more of the symptoms associated with the abnormal condition; and (e) enhancing the function of the affected population of cells.

An “abnormal condition” refers to a function in the cells or tissues of an organism that deviates from their normal functions in that organism. An abnormal condition can relate to, for example without limitation, cell proliferation, cell differentiation, or cell survival. Abnormal cell proliferative conditions include, for example, cancers such as fibrotic and mesangial disorders, abnormal angiogenesis and vasculogenesis, wound healing, psoriasis, diabetes mellitus, and inflammation. Abnormal differentiation conditions include, but are not limited to neurodegenerative disorders, slow wound healing rates, and slow tissue grafting healing rates. Abnormal cell survival conditions relate to, for example without limitation, conditions in which programmed cell death (apoptosis) pathways are activated or abrogated. A number of proteases are associated with the apoptosis pathways.

The abnormal condition can be prevented or treated with an identified test compound or novel protease of the invention when the cells or tissues of the organism exist within the organism or outside of the organism. Cells existing outside the organism can be maintained or grown in cell culture dishes. For cells harbored within the organism, many techniques exist in the art to administer compounds, including (but not limited to) oral, parenteral, dermal, injection, and aerosol applications. For cells outside of the organism, multiple techniques exist in the art to administer the compounds, including (but not limited to) cell microinjection techniques, transformation techniques, and carrier techniques.

A “functional part,” “functional variant” or “functional fragment” is a portion of a full-length protease of any one of SEQ ID NOs. 1-92 that comprises the amino acid residues required to catalyze hydrolysis of a peptide bond, i.e., residues that convey proteolytic activity upon a protein of SEQ ID NOs. 1-92. SEQ ID NOs. 1.

A “variant” polypeptide of the invention can differ in amino acid sequence from a protease selected from the sequences represented in SEQ ID NOs. 1-92, or a functional variant thereof by one or more substitutions, deletions, insertions, inversions, and truncations or a combination of any of these. Any one of the novel proteases can be made to contain amino acid substitutions that substitute a given amino acid with another amino acid of similar characteristics. See Bowie et al., Science 247:1306-1310 (1990). A “variant,” according to the invention retains protease activity.

The term “polyclonal” refers to antibodies that are heterogenous populations of antibody molecules derived from the sera of animals immunized with an antigen or an antigenic functional derivative thereof. For the production of polyclonal antibodies, various host animals may be immunized by injection with the antigen. Various adjuvants may be used to increase the immunological response, depending on the host species.

“Monoclonal antibodies” are substantially homogenous populations of antibodies to a particular antigen. They may be obtained by any technique which provides for the production of antibody molecules by continuous cell lines in culture. Monoclonal antibodies may be obtained by methods known to those skilled in the art (Kohler et al., Nature, 1 975, 256:495-497, and U.S. Pat. No. 4,376,110, both of which are hereby incorporated by reference herein in their entirety including any figures, tables, or drawings).

The term “antibody fragment” refers to a portion of an antibody, often the hypervariable region and portions of the surrounding heavy and light chains, that displays specific binding affinity for a particular molecule. A hypervariable region is a portion of an antibody that physically binds to the polypeptide target. “Operatively linked” indicates that the inventive protease sequence and the heterologous protein are both in-frame or are chemically attached to each other.

The term “specific binding affinity” describes an antibody that binds to a protease polypeptide with greater affinity than it binds to other polypeptides under specified conditions. Antibodies can be used to identify an endogenous source of protease polypeptides, to monitor cell cycle regulation, and for immuno-localization of protease polypeptides within the cell. They may also be used therapeutically.

The term “antibody fragment” refers to a portion of an antibody, often the hypervariable region and portions of the surrounding heavy and light chains, that displays specific binding affinity for a particular molecule. A hypervariable region is a portion of an antibody that physically binds to the polypeptide target.

An antibody fragment of the present invention includes a “single-chain antibody,” a phrase used in this description to denote a linear polypeptide that binds antigen with specificity and that comprises variable or hypervariable regions from the heavy and light chain chains of an antibody. Such single chain antibodies can be produced by conventional methodology. The Vh and VI regions of the Fv fragment can be covalently joined and stabilized by the insertion of a disulfide bond. See Glockshuber, et al., Biochemistry 1362 (1990). Alternatively, the Vh and VI regions can be joined by the insertion of a peptide linker. A gene encoding the Vh, VI and peptide linker sequences can be constructed and expressed using a recombinant expression vector. See Colcher, et al., J. Nat'l Cancer Inst. 82:1191(1990). Amino acid sequences comprising hypervariable regions from the Vh and VI antibody chains can also be constructed using disulfide bonds or peptide linkers.

The identified serine-, cysteine-, aspartic-, threonine-, and metallo-proteases of the present invention were found to either

(i) share less than 90% sequence identity to known proteases;

(ii) share less than 90% sequence identity to a protein encoded by a gene of known function which is not identified as a protease;

(iii) be identical to a protein product of a gene of unknown function;

(iv) be identical to a protein product of a gene of known function, which is not identified as a protease; or

(v) share less than 90% identity to a protein product of a gene of unknown function.

The proteins of the present invention may be modified, for example, so as to change residues which do not abrogate proteolytic activity. Amino acids that are not critical for function can be identified by methods known in the art, such as site-directed mutagenesis, crystallization, nuclear magnetic resonance, photoaffinity labeling or alanine-scanning mutagenesis (Cunningham et al., Science 244:1081-1085 (1989); Smith et al., J. Mol. Biol. 224:899-904 (1992); de Vos et al. Science 255:306-312 (1992)). Modified proteins can be tested for biological activity such as protease binding to substrate, cleavage, or in vitro, or in vitro activity. Such modifications are described in detail in the art. See, for example, U.S. Pat. No. 6,331,427 to Robison. The proteins of the present invention may also be used for targeted enzyme prodrug therapy (“TEPT”) which are described in U.S. provisional application Ser. Nos. 60/225,774 and 60/279,609, and which are incorporated herein by reference.

As an embodiment of the invention, any one of the proteases can be made to contain amino acid substitutions.

A polypeptide having the full-length sequence of any one of SEQ ID NOs. 1-92, or a functional part thereof, can also be joined to another polypeptide with which it is not normally associated. Thus, a protease amino acid sequence of SEQ ID NOs. 1-92 is operatively linked, at either its N-terminus or C-terminus, or in a side chain, to a heterologous protein having an amino acid sequence not substantially homologous to the protease

A fusion protein may, or may not, affect the protease activity of a protein having a sequence of any one of SEQ ID NOs. 1-92, or a functional part thereof. For example, the fusion protein can be a GST-fusion protein in which the protease sequences are fused to the C-terminus of the GST sequences or an influenza HA marker. Other types of fusion proteins include, but are not limited to, enzymatic fusion proteins, for example beta-galactosidase fusions, yeast two-hybrid GAL fusions, poly-His fusions and Ig fusions. Such fusion proteins, particularly poly-His fusions, can facilitate the purification of protease of the invention. In certain host cells, expression and/or secretion of a protein can be increased by using a heterologous signal sequence fused to a protease of the invention that transports the protease to an extracellular matrix or localizes the protease in the cell membrane.

Other fusion proteins may affect the protease activity of a protein having a sequence of any one of SEQ ID NOs. 1-92, or of a functional part thereof. For example, without limitation, one or more of the protease domains (or parts thereof in any one of SEQ ID NOs. 1-92 may be replaced by domains from another protease or other type of protease. Similarly, a substrate binding, or subregion thereof, can be replaced, for example, with the corresponding domain or subregion from another protease with different substrate specificity. Accordingly, chimeric proteases can be produced from any one of SEQ ID NOs. 1-92, or a functional variant thereof which have altered cleavage characteristics, such that release of substrate is faster or slower than that of the unmodified protease or sequence recognized by the protease is altered Likewise, the affinity for substrate can be altered or even proteolysis of the substrate prevented. Non-functional variants of SEQ ID NOs. 1-92 may be engineered to contain one or more amino acid substitutions, deletions, insertions, inversions, or truncations in a critical residue or critical region. Modifications can be made to SEQ ID NOs. 1-92 to affect the function, for example, of one or more of the regions corresponding to substrate binding, subcellular localization (such as membrane association), proteolytic cleavage or effector binding.

Biologically active fragments of SEQ ID NOs. 1-92 can comprise a domain or region identified by analysis of the polypeptide sequence by well-known methods, Such biologically active fragments include, but are not limited to domains comprising one or more cleavage sites, substrate binding sites, glycosylation sites, cAMP and cGMP-dependent phosphorylation sites, N-myristoylation sites, activator binding sites, casein kinase II phosphorylation sites, palmitoylation sites, amidation sites. Such domains or sites can be identified by means of routine procedures for computerized homology or motif analysis.

Variants of the polypeptides of the invention having the sequences described in SEQ ID NOs. 1-92 also encompass derivatives or analogs in which (i) an amino acid is substituted with an amino acid residue that is not one encoded by the genetic code, (ii the mature polypeptide is fused with another compound, such as a compound to increase the half-life of the polypeptide (for example, polyethylene glycol), or (iii) additional amino acids are fused to the mature polypeptide, such as a leader or secretory sequence or a sequence for purification of the mature polypeptide or a pro-protein sequence. Known modifications include, but are not limited to, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

Particularly common modifications include glycosylation, lipid attachment, sulfation, gamma-carboxylation of glutamic acid residues, hydroxylation and ADP-ribosylation. See PROTEINS—STRUCTURE AND MOLECULAR PROPERTIES, 2nd Ed., T. E. Creighton, W. H. Freeman and Company, New York (1993); Wold, F., POSTTRANSLATIONAL COVALENT MODIFICATION OF PROTEINS, B. C. Johnson, Ed., Academic Press, New York 1-12 (1983); Seifter et al. (Meth. Enzymol. 182: 626-646 (1990)) and Rattan et al. (Ann. N.Y. Acad. Sci. 663:48-62 (1992)).

Modifications can be made anywhere in a polypeptide, including the peptide backbone, the amino acid side-chains and the amino or carboxyl termini. Blockage of the amino or carboxyl group in a polypeptide, or both, by a covalent modification, is common in naturally-occurring and synthetic polypeptides.

A protease of the present invention may be modified by the process in which it is synthesized. With recombinantly-produced polypeptides, for example, the modifications will be determined by the host cell post-translational modification capacity and the modification signals in the polypeptide amino acid sequence. Accordingly, when glycosylation is desired, a polypeptide should be expressed in a glycosylating host, generally a eukaryotic cell. The same type of modification may be present in the same or varying degree at several sites in a given polypeptide. Also, a given polypeptide may contain more than one type of modification.

The protein sequences of SEQ ID NOs. 1-92, or a functional variant thereof, can be used to identify compounds that modulate protease activity. Such compounds may increase or decrease affinity or rate of binding to a substrate or activator, compete with substrate or activator for binding to the protease or displace substrate or activator bound to the protease. For instance, a compound may be a mutated protease or a functional variant thereof, or appropriate fragments containing mutations that compete for substrate, activator or other protein that interacts with the protease. Accordingly, a fragment that competes for substrate or activator, for example with a higher affinity, or a fragment that binds substrate or activator but does not allow release, is encompassed by the invention.

Thus, compounds that activate or inactivate or bind to (i.e., “modulate”) a protease having a primary amino acid sequence described in SEQ ID NOs. 1-92 of the instant invention can be identified by a simple screening assay.

According to the present invention, the newly identified protease protein can be used in an assay for screening for a compound that modulates the activity of a protein which comprises the steps of (i) combining a protease having a sequence of any one of SEQ ID NOs. 1-92, or a functional variant thereof with a test compound and substrate and (ii) detecting a biochemical change in an interaction between the protease and the substrate in the presence and absence of the test compound.

The activity of the novel proteases can be determined by examining the ability to cleave substrate in the presence of chemically synthesized peptide ligands. Thus, modulators of the protease polypeptide 's activity may, among other things, alter a protease function, such as a binding property of a protease for a natural or synthetic substrate or inhibitor, or an activity such as cleaving protein or polypeptide substrates, membrane localization, processing the pro-form of a polypeptide chain to the active product, transmembrane signaling of various forms, and/or the modification of the extracellular matrix or small molecule fluorescent substrate. (see, for example, THE HANDBOOK OF PROTEOLYTIC ENZYMES, 1998, Academic Press, San Diego, which is hereby incorporated by reference, including any drawings).

According to the assays of the present invention, one of skill in the art may determine the effect, if any, of the test compound upon proteolytic cleavage; upon a cellular response, such as development, differentiation, apoptosisor rate of proliferation; or upon a change in substrate levels. An indicator of a compound's ability to modulate a protease of the invention may be measured by parameters other than those intrinsic to the function of the specific protease. A screening assay may also involve monitoring biological events that are affected by the action of the test compound, such as, for example, when the action of a pathway in which the protease functions, or is made to function, that indicate protease activity. Thus, the expression or activity of genes that are up- or down-regulated in response to a protease-dependent cascade can be assayed.

A screening assay of the invention may also expose a test compound to some or all of the proteases of the invention to determine the specificity of the compound in modulating the novel proteases. The present invention is particularly useful for screening compounds by using a protease polypeptide in any of a variety of drug screening techniques. The compounds to be screened include, but are not limited to, extracellular, intracellular, biological or chemical origin. The protease polypeptide employed in such a test may be in any form, such as free in solution, attached to a solid support, borne on a cell surface or located intracellularly. One skilled in the art can measure the change in rate that a protease of the invention cleaves a substrate (See, for example, THE HANDBOOK OF PROTEOLYTIC ENZYMES, 1998, Academic Press, San Diego.) One skilled in the art can also, for example, measure the formation of complexes between a protease polypeptide and the compound being tested. Alternatively, one skilled in the art can examine the diminution in complex formation between a protease polypeptide and its substrate caused by the compound being tested.

Examples of assays include, but are not limited to, a yeast growth assay, an Aequorin assay, a Luciferase assay, a mitogenesis assay, a quench fluorescent substrate cleavage assay, as well as other binding and/or catalytic function-based assays of protease activity that are generally known in the art. See, for example, THE HANDBOOK OF PROTEOLYTIC ENZYMES, 1998, Academic Press, San Diego.

The use of cDNAs encoding proteins in drug discovery programs is well-known. Assays capable of testing thousands of unknown compounds per day in high-throughput screens (HTSs) are thoroughly documented. The literature is replete with examples of the use of enzymatic assays in HTS binding assays for drug discovery (see, Williams, Medicinal Research Reviews, 1991, 11:147-184.; Sweetnam, et al., J. Natural Products, 1993, 56:441-455 for review). Recombinant proteins are preferred for enzymatic binding assay HTS because they allow for better specificity (higher relative purity), provide the ability to generate large amounts of material, and can be used in a broad variety of formats (see Hodgson, Bio/Technology, 1992, 10:973-980 which is incorporated herein by reference in its entirety). To this end, a variety of heterologous systems is available for functional expression of recombinant proteins that are well known to those skilled in the art. Such systems include bacteria (Strosberg, et al., Trends in Pharmacological Sciences, 1992, 13:95-98), yeast (Pausch, Trends in Biotechnology, 1997, 15:487-494), several kinds of insect cells (Vanden Broeck, Int. Rev. Cytology, 1996, 164:189-268), amphibian cells (Jayawickreme et al., Current Opinion in Biotechnology, 1997, 8:629-634) and several mammalian cell lines (CHO, HEK293, COS, etc.; see, Gerhardt, et al., Eur. J. Pharmacology, 1997, 334:1-23). These examples do not preclude the use of other possible cell expression systems, including cell lines obtained from nematodes (PCT application WO 98/37177).

The invention also contemplates production of the protease. The invention further includes a method for producing a protease having an amino acid sequence selected from the group consisting of SEQ ID NOs: 1-92 by recombinant techniques, by culturing recombinant prokaryotic or eukaryotic host cells comprising nucleic acid sequence encoding said protease under conditions effective to promote expression of the protein, and subsequent recovery of the protein from the host cell or the cell culture medium.

Foreign protein production, including the production and secretion of mammalian proteins, has been reported previously in filamentous fungi. See U.S. Pat. Nos. 6,103,490, 5,840,570, 5,679,543 and 5,364,770.

The invention also contemplates the ability of determining whether a protease can bind to a substrate, inhibitor or other molecule can also be determined by real-time Bimolecular Interaction Analysis (BIA). Sjolander, S. and Urbaniczky, C. (1991) Anal. Chem., 63:2338-2345 and Szabo et al. (1995) Curr. Opin. Struct. Biol., 5:699-705. “BIA” is a technology for studying biospecific interactions in real time, without labeling any of the interactants. Changes in the optical phenomenon surface plasmon resonance (SPR) can be used as an indication of real-time reactions between biological molecules. Similarly, a microphysiometer can be used to detect the interaction of a test compound with the polypeptide without the labeling of either the test compound or the polypeptide. McConnell, H. M. et al. (1992) Science, 257:1906-1912.

The proteins of SEQ ID NOs. 1-92 can also be used in a two-hybrid assay or three-hybrid assay (see, e.g., U.S. Pat. No. 5,283,317; Zervos et al. (1993) Cell, 72:223-232; Madura et al. (1993) J. Biol. Chem., 268:12046-12054; Bartel et al. (1993) Biotechniques, 14:920-924; Iwabuchi et al. (1993) Oncogene, 8:1693-1696; and Brent W094/10300), to identify other proteins which bind to or interact with the proteins of the invention and modulate their activity.

Binding can be determined by binding assays which are well known to the skilled artisan, including, but not limited to, gel-shift assays, Western blots, radiolabeled competition assay, phage-based expression cloning, co-fractionation by chromatography, co-precipitation, cross linking, interaction trap/two-hybrid analysis, southwestern analysis, ELISA, and the like, which are described in, for example, Current Protocols in Molecular Biology, 1999, John Wiley & Sons, New York, which is incorporated herein by reference in its entirety. The compounds to be screened include, but are not limited to, compounds of extracellular, intracellular, biological or chemical origin.

Other assays can be used to examine enzymatic activity including, but not limited to, photometric, radiometric, HPLC, electrochemical, and the like, which are described in, for example, ENZYME ASSAYS: A PRACTICAL APPROACH, eds. R. Eisenthal and M. J. Danson, 1992, Oxford University Press, which is incorporated herein by reference in its entirety.

Test compounds of the present invention can be obtained, for example, without limitation, from biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to polypeptide libraries, while the other four approaches are applicable to polypeptide, non-peptide oligomer or small molecule libraries of compounds (Lam, K. S. (1997) Anticancer Drug Des. 12:145). Examples of methods for the synthesis of molecular libraries can be found in the art, for example in DeWitt et al. (1993) Proc. Natl. Acad. Sci. U.S.A., 90:6909; Erb et al. (1994) Proc. Natl Acad. Sci. U.S.A., 91:11422; Zuckermann et al. (1994). J. Med. Chem., 37:2678; Cho et al.(1993) Science, 261:1303; Carell et al. (1994) Angew. Chem. Int. Ed. Engl., 33:2059; Carell et al. (1994) Angew. Chem. Int. Ed. Engl., 33:2061; and in Gallop et al. (1994) J. Med. Chem., 37:1233.

The invention does not restrict the sources for suitable test compounds, which may be obtained from natural sources such as plant, animal or mineral extracts, or non-natural sources such as small molecule libraries, including the products of combinatorial chemical approaches to library construction, and peptide libraries.

Libraries of compounds may be presented in solution (e.g., Houghten (1992) Biotechniques, 13:412-421), or on beads (Lam(1991) Nature, 354:82-84), chips (Fodor (1993) Nature, 364;555-556), bacteria (Ladner U.S. Pat. No. 5,223,409), spores (Ladner U.S. Pat. No. '409), plasmids (Cull et al. (1992) Proc. Natl. Acad. Sci. U.S.A., 89:1865-1869) or on phage (Scott and Smith (1990) Science, 249:386-390); (Devlin (1990) Science, 249:404-406); (Cwirla et al. (1990) Proc. Natl. Acad. Sci., 97:6378-6382); (Felici (1991) J. Mol. Biol., 222:301-310); (Ladner supra or a library of mammalian cells Test compounds include, for example, peptides such as soluble peptides, including Ig-tailed fusion peptides and members of random peptide libraries (see, e.g., Lam et al., Nature 354:82-84 (1991); Houghten et al., Nature 354:84-86 (1991)) and combinatorial chemistry-derived molecular libraries made of D- and/or L-configuration amino acids; phosphopeptides (e.g., members of random and partially degenerate, directed phosphopeptide libraries, see, e.g., Songyang et al., Cell 72:767-778 (1993)); antibodies (e.g., polyclonal, monoclonal, humanized, anti-idiotypic, chimeric, and single chain antibodies as well as Fab, F(ab′)₂, Fab expression library fragments, and epitope-binding fragments of antibodies); and small organic and inorganic molecules such as those obtained from combinatorial and natural product libraries. Preferably, these inhibitors will have molecular weights from 100 to 200 daltons, from 200 to 300 daltons, from 300 to 400 daltons, from 400 to 600 daltons, from 600 to 1000 daltons, from 1000 to 2000 daltons, from 2000 to 4000 daltons, from 4000 to 8000 daltons and from 8000 to 60 daltons.

The test compound may also be a drug or a chemical. Examples of such compounds include, but are not limited to, phenylmethylsulfonyl fluoride (PMSF), diisopropylfluorophosphate (DFP) (chapter 3, Barrett et al., Handbook of Proteolytic Enzymes, 1998, Academic Press, San Diego), 3,4-dichloroisocoumarin (DCI) (Id., chapter 16), serpins (Id., chapter 37), E-64 (trans-epoxysuccinyl L-leucylamido-(4-guanidino) butane) (Id., chapter 188), peptidyl-diazomethanes, peptidyl-O-acyl-hydroxamates, epoxysuccinyl-peptides (Id., chapter 210), DAN, EPNP (1,2-epoxy-3(p-nitrophenoxy)propane) (Id., chapter 298), thiorphan (dl-3-Mercapto-2-benzylpropanoyl-glycine) (Id., chapter 362), CGS 26303, PD 069185 (Id., chapter 363), and COT989-00 (N-4-hydroxy-N1-[1-(s)-(4-aminosulfonyl)phenylethylaminocarboxyl-2-cyclohexylethyl)-2R-[4-methyl)phenylpropyl]succinamide) (Id., chapter 401). Other protease inhibitors include, but are not limited to, aprotinin, amastatin, antipain, calcineurin autoinhibitory fragment, and histatin 5 (Id.). Compounds that can traverse cell membranes and are resistant to acid hydrolysis are potentially advantageous as therapeutics as they can become highly bioavailable after being administered orally to patients.

Compounds identified through such screening assays that modulate the activity of a protein having a sequence described in any one of SEQ ID NOs. 1-92, or a functional variant thereof can be used to treat a subject with a disorder mediated by a protease pathway, by treating cells that express the protease. These methods of treatment include the steps of administering the compound(s) that modulate activity, for example in a pharmaceutical composition to a subject in need of such treatment.

Alternatively, or in conjunction, a protease of SEQ ID NOs. 1-92 may be therapeutically administered to a subject in need of such treatment in a pharmaceutical composition. Such substances, useful for treatment of protease-related disorders or diseases, preferably show positive results in one or more in vitro assays for an activity corresponding to treatment of the disease or disorder in question.

A compound identified according to an assay described herein, or a protein having a sequence of any one of SEQ ID NOs. 1-92, or a functional variant thereof may be administered to an individual to compensate for reduced or aberrant expression or activity of an endogenous protein in vivo. Accordingly, methods for treatment include the use of soluble protease or fragments of the protease protein that compete, for example, with activator or substrate binding. These proteases or fragments can have a higher affinity for the activator or substrate so as to provide effective competition.

The compound(s) and protease(s) or variants thereof, can be administered to a human patient directly, or in the form of a pharmaceutical composition, admixed with other active ingredients, as in combination therapy, or suitable carriers or excipient(s). Techniques for formulation and administration of the compounds of the instant application may be found in REMINGTON'S PHARMACEUTICAL SCIENCES, Mack Publishing Co., Easton, Pa., latest edition. All methods are well-known in the art.

Many of the protease modulating compounds of the invention may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents that are the corresponding free base forms.

Pharmaceutical compositions suitable for use in the present invention include compositions where the active ingredients, i.e., a compound identified from a screening assay described herein, or any one of the novel proteases having a sequence described in SEQ ID NOs. 1-92, or a functional variant thereof, are contained in an amount effective to achieve its intended purpose. More specifically, a therapeutically effective amount of a compound or novel protease means an amount of compound effective to prevent, alleviate or ameliorate symptoms of disease or prolong the survival of the subject being treated. Determination of a therapeutically effective amount is well within the capability of those skilled in the art, especially in light of the detailed disclosure provided herein.

A protease of the present invention may also be used as a diagnostic marker of a disease or disorder. One may compare a nucleic acid target obtained from an individual that encodes a protease of SEQ ID NOs. 1-92, or a functional variant thereof with that of a control nucleic acid target encoding the protease; and then (b) detecting differences in sequence or amount between the target region and the control target region, as an indication of said disease or disorder. A method for detecting a protease in a sample as a diagnostic marker of a disease or disorder may comprise (a) contacting the sample with a nucleic acid probe which hybridizes under hybridization assay conditions to a nucleic acid target encoding a protease having an amino acid sequence selected from the group consisting of SEQ ID NOs 1-92, or a functional variant thereof or the complements of said sequences and fragments thereof; and (b) detecting the presence or amount of the probe:nucleic acid target region hybrid as an indication of the disease.

Methods for using nucleic acid probes include detecting the presence or amount of protease RNA in a sample by contacting the sample with a nucleic acid probe under conditions such that hybridization occurs and detecting the presence or amount of the probe bound to protease RNA. The nucleic acid duplex formed between the probe and a nucleic acid sequence coding for a protease polypeptide may be used in the identification of the sequence of the nucleic acid detected (Nelson et al., in NONISOTOPIC DNA PROBE TECHNIQUES, Academic Press, San Diego, Kricka, ed., p. 275, 1992, hereby incorporated by reference herein in its entirety, including any drawings, figures, or tables). In another aspect, the invention describes a recombinant cell or tissue comprising a nucleic acid molecule encoding a protease polypeptide having an amino acid sequence selected from the group consisting of those set forth in SEQ ID NOs. 1-92, or a functional variant thereof. Accordingly, such a cell or tissue may be grown or differentiated and introduced into an individual in need of treatment. In such fashion, the novel protease may be introduced into an individual by cellular administration of cells or tissues, rather than by direct injection. Accordingly, cells or tissues may be taken from the individual in question, modified so as to contain cells expressing a protease of any one of SEQ ID NOs. 1-92, or a functional variant thereof and then reintroduced into the same individual. Mesenchymal stem cells and bone marrow stem cells are examples of cells that may be modified and used in such fashion.

The novel proteases will be useful for screening for compounds that modulate (e.g., activate or inhibit) the catalytic activity of the encoded protease with potential utility in treating cancers, immune-related diseases and disorders, cardiovascular disease, brain or neuronal-associated diseases, and metabolic disorders. More specifically disorders including cancers of tissues, blood, or hematopoietic origin, particularly those involving breast, colon, lung, prostate, cervical, brain, ovarian, bladder, or kidney; central or peripheral nervous system diseases and conditions including migraine, pain, sexual dysfunction, mood disorders, attention disorders, cognition disorders, hypotension, and hypertension; psychotic and neurological disorders, including anxiety, schizophrenia, manic depression, delirium, dementia, severe mental retardation and dyskinesias, such as Huntington's disease or Tourette's Syndrome; neurodegenerative diseases including Alzheimer's, Parkinson's, multiple sclerosis, and amyotrophic lateral sclerosis; viral or non-viral infections caused by HIV-1, HIV-2 or other viral- or prion-agents or fungal- or bacterial-organisms; metabolic disorders including Diabetes and obesity and their related syndromes, among others; cardiovascular disorders including reperfusion restenosis, coronary thrombosis, clotting disorders, unregulated cell growth disorders, atherosclerosis; ocular disease including glaucoma, retinopathy, and macular degeneration; inflammatory disorders including rheumatoid arthritis, chronic inflammatory bowel disease, chronic inflammatory pelvic disease, multiple sclerosis, asthma, osteoarthritis, psoriasis, atherosclerosis, rhinitis, autoimmunity, and organ transplant rejection.

Antibody generation

The protein sequences of SEQ ID NOs. 1-92 are also useful for producing antibodies specific for the protease, regions, or fragments. The antibody preferably binds to the target protease polypeptide with greater affinity than it binds to other inhibitor polypeptides under specified conditions. Antibodies or antibody fragments are polypeptides that contain regions that can bind other polypeptides. An antibody or antibody fragment with specific binding affinity to a protease polypeptide of the invention can be isolated, enriched, or purified from a prokaryotic or eukaryotic organism. Routine methods known to those skilled in the art enable production of antibodies or antibody fragments, in both prokaryotic and eukaryotic organisms. Purification, enrichment, and isolation of antibodies, which are polypeptide molecules, are described above.

Antibodies having specific binding affinity to a protease of the invention may be used in methods for detecting the presence and/or amount of protease polypeptide in a sample by contacting the sample with the antibody under conditions such that an immunocomplex forms and detecting the presence and/or amount of the antibody conjugated to the protease polypeptide. In another aspect, the invention features an antibody (e.g., a monoclonal or polyclonal antibody) having specific binding affinity to a protease polypeptide or a protease polypeptide domain or fragment where the polypeptide is selected from the group having a sequence at least about 90% identical to an amino acid sequence selected from the group consisting of those set forth in SEQ ID NO:1-92. Preferably the polypeptide is has at least about 90%, 91%, 92%, 93%, 94%, 95%, 96%, 97%, 98% 99% or 100% identity with the sequences listed above. By “specific binding affinity” is meant that the antibody binds to the target protease polypeptide with greater affinity than it binds to other polypeptides under specified conditions. Antibodies or antibody fragments are polypeptides that contain regions that can bind other polypeptides. The term “specific binding affinity” describes an antibody that binds to a protease polypeptide with greater affinity than it binds to other polypeptides under specified conditions. Antibodies can be used to identify an endogenous source of protease polypeptides, to monitor cell cycle regulation, and for immuno-localization of protease polypeptides within the cell.

An antibody of the present invention includes “humanized” monoclonal and polyclonal antibodies. Humanized antibodies are recombinant proteins in which non-human (typically murine) complementarity determining regions of an antibody have been transferred from heavy and light variable chains of the non-human (e.g. murine) immunoglobulin into a human variable domain, followed by the replacement of some human residues in the framework regions of their murine counterparts. Humanized antibodies in accordance with this invention are suitable for use in therapeutic methods. General techniques for cloning murine immunoglobulin variable domains are described, for example, by the publication of Orlandi et al., Proc. Nat'l Acad. Sci. USA 86: 3833 (1989). Techniques for producing humanized monoclonal antibodies are described, for example, by Jones et al., Nature 321:522 (1986), Riechmann et al., Nature 332:323 (1988), Verhoeyen et al., Science 239:1534 (1988), Carter et al., Proc. Nat'l Acad. Sci. USA 89:4285 (1992), Sandhu, Crit. Rev. Biotech. 12:437 (1992), and Singer et al., J. Immun. 150:2844 (1993).

Antibodies or antibody fragments having specific binding affinity to a protease polypeptide of the invention may be used in methods for detecting the presence and/or amount of protease polypeptide in a sample by probing the sample with the antibody under conditions suitable for protease-antibody immunocomplex formation and detecting the presence and/or amount of the antibody conjugated to the protease polypeptide. Diagnostic kits for performing such methods may be constructed to include antibodies or antibody fragments specific for the protease as well as a conjugate of a binding partner of the antibodies or the antibodies themselves.

An antibody or antibody fragment with specific binding affinity to a protease polypeptide of the invention can be isolated, enriched, or purified from a prokaryotic or eukaryotic organism. Routine methods known to those skilled in the art enable production of antibodies or antibody fragments, in both prokaryotic and eukaryotic organisms. Purification, enrichment, and isolation of antibodies, which are polypeptide molecules, are described above.

Antibodies having specific binding affinity to a protease polypeptide of the invention may be used in methods for detecting the presence and/or amount of protease polypeptide in a sample by contacting the sample with the antibody under conditions such that an immunocomplex forms and detecting the presence and/or amount of the antibody conjugated to the protease polypeptide. Diagnostic kits for performing such methods may be constructed to include a first container containing the antibody and a second container having a conjugate of a binding partner of the antibody and a label, such as, for example, a radioisotope. The diagnostic kit may also include notification of an FDA approved use and instructions therefor.

In another aspect, the invention features a hybridoma which produces an antibody having specific binding affinity to a protease polypeptide or a protease polypeptide domain, where the polypeptide is selected from the group consisting of those set forth in any one of SEQ ID Nos 1-92.

Table 1 shows each of the ninety-two proteins according to their protease family and percent sequence similarity to known and unknown proteins. None of the proteases are described in publicly available protein databases as possessing protease activity (i e., as having protease activity or are used as proteases).

Table 2 shows the beginning and end of the active domain for each of the proteases having a sequence described in SEQ ID NOS: 1-92. A functional variant of one of SEQ ID NOs. 1-92 can be determined in reference to Table 2. For example, one skilled in the art could use a delimited domain, as determined by multiple alignments, to determine which part of a sequence has catalytic activity and is therefore a functional variant, in spite of the fact that the sequences are not full-length sequences.

TABLE 1
Classification of novel proteases

| Cysteine peptidase | Serine peptidase | Aspartic peptidase | Threonine peptidase | Metallopeptidase |
|---|---|---|---|---|
| <90% identity to known | <90% identity to known | <90% identity to known | Identical to gene of | Identical to gene of |
| protease | protease | protease | unknown function | unknown function |
| SEQ ID NO. 3 | SEQ ID NO. 4 | SEQ ID NO. 1 | SEQ ID NO. 12 | SEQ ID NO. 15 |
| Identical to gene of | SEQ ID NO. 5 | SEQ ID NO. 2 | SEQ ID NO. 23 | |
| unknown function | | | | |
| SEQ ID NO. 10 | Identical to gene of | SEQ ID NO. 6 | Identical to a gene of | |
| | unknown function | | known function (non- | |
| | | | protease) | |
| SEQ ID NO. 17 | SEQ ID NO. 11 | <90% identity to known | SEQ ID NO. 32 | |
| | | gene of known function | | |
| | | (non-protease) | | |
| SEQ ID NO. 18 | SEQ ID NO. 13 | SEQ ID NO. 7 | SEQ ID NO. 45 | |
| SEQ ID NO. 19 | SEQ ID NO. 16 | SEQ ID NO. 8 | SEQ ID NO. 53 | |
| SEQ ID NO. 25 | SEQ ID NO. 20 | SEQ ID NO. 9 | <90% identity to gene of | |
| | | | unknown function | |
| SEQ ID NO. 29 | SEQ ID NO. 21 | Identical to gene of | SEQ ID NO. 90 | |
| | | unknown function | | |
| Identical to a gene of | SEQ ID NO. 22 | SEQ ID NO. 14 | | |
| known function (non- | | | | |
| protease) | | | | |
| SEQ ID NO. 30 | SEQ ID NO. 24 | Identical to a gene of | | |
| | | known function (non- | | |
| | | protease) | | |
| SEQ ID NO. 33 | SEQ ID NO. 26 | SEQ ID NO. 35 | | |
| SEQ ID NO. 34 | SEQ ID NO. 27 | SEQ ID NO. 41 | | |
| SEQ ID NO. 37 | SEQ ID NO. 28 | SEQ ID NO. 43 | | |
| SEQ ID NO. 38 | Identical to a gene of known | SEQ ID NO. 47 | | |
| | function (non-protease) | | | |
| SEQ ID NO. 42 | SEQ ID NO. 31 | SEQ ID NO. 49 | | |
| SEQ ID NO. 44 | SEQ ID NO. 36 | SEQ ID NO. 52 | | |
| SEQ ID NO. 51 | SEQ ID NO. 39 | SEQ ID NO. 60 | | |
| SEQ ID NO. 55 | SEQ ID NO. 40 | SEQ ID NO. 70 | | |
| SEQ ID NO. 56 | SEQ ID NO. 46 | SEQ ID NO. 71 | | |
| SEQ ID NO. 57 | SEQ ID NO. 48 | SEQ ID NO. 74 | | |
| SEQ ID NO. 62 | SEQ ID NO. 50 | SEQ ID NO. 75 | | |
| SEQ ID NO. 63 | SEQ ID NO. 54 | SEQ ID NO. 76 | | |
| SEQ ID NO. 66 | SEQ ID NO. 58 | SEQ ID NO. 78 | | |
| SEQ ID NO. 67 | SEQ ID NO. 59 | SEQ ID NO. 82 | | |
| SEQ ID NO. 68 | SEQ ID NO. 61 | <90% identity to gene of | | |
| | | unknown function | | |
| SEQ ID NO. 69 | SEQ ID NO. 64 | SEQ ID NO. 83 | | |
| SEQ ID NO. 72 | SEQ ID NO. 65 | SEQ ID NO. 86 | | |
| SEQ ID NO. 77 | SEQ ID NO. 73 | SEQ ID NO. 88 | | |
| SEQ ID NO. 80 | SEQ ID NO. 79 | SEQ ID NO. 91 | | |
| SEQ ID NO. 81 | <90% identity to gene of | SEQ ID NO. 92 | | |
| | unknown function | | | |
| <90% identity to gene of | SEQ ID NO. 85 | | | |
| unknown function | | | | |
| SEQ ID NO. 87 | SEQ ID NO. 89 | | | |

TABLE 2
Regions demarcating the active domain of each novel protease

| | Residue number | Residue number |
|---|---|---|
| Protease | marking the start of | marking the end of |
| SEQ ID NO.: | the active domain | the active domain |
| 1 | 104 | 231 |
| 2 | 66 | 360 |
| 3 | 1 | 122 |
| 4 | 3 | 393 |
| 5 | 15 | 153 |
| 6 | 235 | 396 |
| 7 | 117 | 294 |
| 8 | 164 | 303 |
| 9 | 384 | 613 |
| 10 | 76 | 271 |
| 11 | 36 | 240 |
| 12 | 234 | 403 |
| 13 | 56 | 371 |
| 14 | 1 | 108 |
| 15 | 258 | 457 |
| 16 | 59 | 285 |
| 17 | 637 | 780 |
| 18 | 44 | 227 |
| 19 | 97 | 292 |
| 20 | 6 | 217 |
| 21 | 118 | 305 |
| 22 | 1 | 239 |
| 23 | 92 | 227 |
| 24 | 26 | 166 |
| 25 | 192 | 711 |
| 26 | 148 | 425 |
| 27 | 294 | 476 |
| 28 | 51 | 298 |
| 29 | 175 | 328 |
| 30 | 2 | 545 |
| 31 | 149 | 761 |
| 32 | 593 | 1829 |
| 33 | 722 | 914 |
| 34 | 687 | 884 |
| 35 | 181 | 346 |
| 36 | 120 | 282 |
| 37 | 411 | 586 |
| 38 | 258 | 444 |
| 39 | 49 | 236 |
| 40 | 500 | 741 |
| 41 | 889 | 1101 |
| 42 | 648 | 836 |
| 43 | 106 | 318 |
| 44 | 988 | 1252 |
| 45 | 1 | 648 |
| 46 | 22 | 558 |
| 47 | 304 | 433 |
| 48 | 137 | 411 |
| 49 | 414 | 492 |
| 50 | 84 | 382 |
| 51 | 243 | 354 |
| 52 | 21 | 130 |
| 53 | 19 | 442 |
| 54 | 158 | 445 |
| 55 | 650 | 838 |
| 56 | 470 | 528 |
| 57 | 698 | 909 |
| 58 | 22 | 270 |
| 59 | 741 | 923 |
| 60 | 68 | 261 |
| 61 | 140 | 385 |
| 62 | 30 | 170 |
| 63 | 564 | 679 |
| 64 | 154 | 707 |
| 65 | 110 | 413 |
| 66 | 1067 | 1190 |
| 67 | 1078 | 1357 |
| 68 | 304 | 558 |
| 69 | 650 | 838 |
| 70 | 138 | 402 |
| 71 | 34 | 297 |
| 72 | 493 | 668 |
| 73 | 42 | 333 |
| 74 | 124 | 388 |
| 75 | 13 | 240 |
| 76 | 54 | 260 |
| 77 | 184 | 294 |
| 78 | 130 | 409 |
| 79 | 13 | 254 |
| 80 | 1113 | 1298 |
| 81 | 412 | 598 |
| 82 | 673 | 864 |
| 83 | 227 | 378 |
| 84 | 137 | 411 |
| 85 | 288 | 465 |
| 86 | 18 | 120 |
| 87 | 1 | 126 |
| 88 | 1 | 124 |
| 89 | 154 | 288 |
| 90 | 108 | 285 |
| 91 | 117 | 294 |

LIST 1

SEQ ID NO. 1

NMAILCANVVGVGLIAGLAVGLTRSCDSSGDGGLGTVPAPSHLPSSTASPSGPPAQDQDICPSSEDESGQWKNFTAE
LRQPGPDLHVKPLLEEDTYTGTVSISINLSTPTRHLWLHLGESRITWLPDTRHLWLHLQETRITWLPEMKRPSGDQV
QIRRCFEYKKQEYVVVEAEEESGDGLYLLTMEFAGWLNSSLLGFTYTENGQVKSIAATDHEPTDARKFFPCFDKPNK
KATYTISVTHPKEYEALSHMPVAKEESVDDKWNQTTFKKSVPMSMYLVCFAVHQFHTVKTISDIGKPVSLII

SEQ ID NO. 2

MSPPLLLLPLLLLLPLLNVEPAGATLDPVWIPLRQVHPGRRTLNLLRGWGKPAELPKLGPSPGDKPASVPLSKFLDA
QYFGEIGLGTPPQNFTVAFDTGSSNLWVPSRRCHFFSVPCWFHHRFNPNASSSFKPSGTKFAIQYGTGRVDGILSED
KLTIGGIKGASVIFGEALWESSLVFTVSRPDGILRLGFPILSVEGVRPPLDVLVEQGLLDKPVFSFYFNRCWGGGXG
CAMYCRIVRLEDPLDTGTPVIVGPTEEIGPCMQPLGESLLAGEYIIRCSEIPKLPAVSLLIGGVWFNLTAQDYVIQF
AQGDVRLCLSGFRALDIASPPVPVWILGDVFLGAYVTVFDRGDMKSGARVGLARARPGADLGRRETQAQYRGCRPGD
HAHRVALALLSKNPIFPLNEPA

SEQ ID NO. 3

MDTAAKAIILEQSGKNQGYRDADIRSFWPEGGVCLPGSPDVLESGVCMKAVCKRVAVEGVDVIFSRDAGRYVCDYTY
YLSLHHGKGCAALIHVPPLSRGLPASLLGRALRVIIQEMLEEVGK

SEQ ID NO. 4

IYVSSWAVQVSQGNREVERLARKFGFVNLGPIFPDGQYFHLRHRGVVQQSLTPHWGHRLHLKKNPKVQWFQQQTLQR
RVKRSVVVPTDPWFSKQWYMNSEAQPDLSILQAWSQGLSGQGIVVSVLDDGIEKDHPDLWANYDPLASYDFNDYDPD
PQPRYTPSKENRHGTRCAGEVAANANNGFCGVGVAFNARIGGVRMLDGTITDVIEAQSLSLQPQHIHIYSASWGPED
DGRTVDGPGILTREAFRRGVTKGRGGLGTLFIWASGNGGLHYDNCNCDGYTNSIHTLSVGSTTQQGRVPWYSEACAS
TLTTTYSSGVATDPQIVTTDLHHGCTDQHTGTSASAPLAAGMIALALEANPFLTWRDMQHLVVRASKPAHLQAEDWR
TNGVGRQG

SEQ ID NO. 5

LVGYAIQYGCIAHCASEYVGGVVMCSGPSMEPTIQNSDTVFAQNLSRHFDSIQRGDIVIAKSPSDPTSNICKRVTGL
EGDKILTTSPSDFFKSYSYYPVGHVWLEGDLQNSTDSSYYGPIPYELIRGRIFFIRPLSDFGFLCASLNGHRFSDD

SEQ ID NO. 6

MLITVYCVRRDLSEVTFSLQVSPDFELRNFKVLCEAESRVPVEEIQIIHMERLLIEDHCSLGSYGLKDGDIVVLLQK
DNVGPRAPGRAPNQPRVDFSGIAVPGTSSSRPQHPGQQQQRTPAAQRSQGLASGEKVAGLQGLGSPALIRSMLLSNP
HDLSLLKERNPPLAEALLSGSLETFSQVLMEQQREKALREQERLRLYTADPLDREAQAKIEEEIRQQNIEENMNIAI
EEAPESFGQVTMLYINCKVNGHPLAKAFVDSGAQMTIMSQACAERCNIMRLVDRRWAGVAKGVGTQRIIGRVHLAQI
QIEGDFLQCSFSILEDQPMDMLLGLDMLRRHQCSIDLKKNVLVIGTTGTQTYFLPEGELPLCSRMVSGQDESSDKEI
THSVNDSGRKEH

SEQ ID NO. 7

HGRVLLPLNLQLGAKVSFVCDEGFRLKGRSASHCVLAGMKALWNSSVPVCERIICGLPPTIANGDFTSISREYFHYG
SVVTYHCNLGSRGKKVFELVGEPSIYCTSKDDQVGIWSGPAPQCIIPNKCTPPNVENGILVSDNRSLFSLNEVVEFR
CQPGFGMKGPSHVKCQALNKWEPELPSCSRVCQPPPDVLHAERTQRDKDNFSPGQEVFYSCEPGYDLRGSTYLHCTP
QGDWSPAAPRCEVKSCDDFLGQLPNGHVLFPLNLQLGAKVDFVCDEGFQLKGSSASYCVLAGMESLWNSSVPVCERK
SCETPPVPVNGMVHVITDIHVGSRINYSCTTGSASHCVLAGTKALWNSSVPVCEQIFCPNPPAILNGRHTGTPPGDI
PYGKEVSYTCDPHPDRGMTFNLIGESTIRRTSEPHGNGVWSSPAPRCELPVGAVIFSTHLITLFYCLGTLLGTIIFI
LIIIFLY

SEQ ID NO. 8

GGGSPGWGCAGIPDSAPGAGVLQAGAVGPARGGQGAEEVGESAGGGEERRVRHPQAPALRLLNRKPQGGSGEIKTPE
NDLQRGRLSRGPRTAPPAPGMGDRSGQQERSVPHSPGAPVGTSAAAVNGLLHNGFHPPPVQPPHVCSRGPVGGSDAA
PQRLPLLPELQPQPLLPQHDSPAKKCRLRRRMDSGRKNRPPFPWFGMDIGGTLVKLVYFEPKDITAEEEQEEVENLK
SIRKYLTSNTAYGKTGIRDVHLELKNLTMCGRKGNLHFIRFPSCAMHRFIQMGSEKNFSSLHTTLCATGGGAFKFEE
DFRMIADLQLHKLDELDCLIQGLLYVDSVGFNGKPECYYFENPTNPELCQKKPYCLDNPYPMLLVNMGSGVSILAVY
SKDNYKRVTGTSLGGGTFLGLCCLLTGCETFEEALEMAAKGDSTNVDKLVKDIYGGDYERFGLQGSAVASSFGNMMS
KEKRDSISKEDLARATLVTITNNIGSIARMCALNENIDRVVFVGNFLRINMVSMKLLAYAMDFWSKGQLKALFLEHE
GYFGAVGALLELFKMTD

SEQ ID NO. 9

MRPVALLLLPSLLALLAHGLSLEAPTVGKGQAPGIEETDGELTAAPTPEQPERGVHFVTTAPTLKLLNHHPLLEEFL
HEGLEKGDEELRPALSFQPDPPAPFTPSALPRLANQDSRPVFTSPTPANGAVPTQPQSKEGPWSPESESPMLRITAP
LPPGPSMAVPTLGPGEIASTTPPSRAWTPTQEGPGDMGRPWVAEVVSQGAGIGIQGTITSSTASGDDEETTTTTTII
TTTITTVQTPGPCSWNFSGPEGSLDSPTDLSSPTDVGLDCFFYISVYPGYGVEIKVQNISLREGETVTVEGLGGPDP
LPLANQSFLLRGQVIRSPTHQAALRFQSLPPPAGPGTFHFHYQAYLLSCHFPRRPAYGDVTVTSLHPGGSARFHCAT
GYQLKGARHLTCLNATQPFWDSKEPVCIAACGGVIRNATTGRIVSPGFPGNYSNNLTCHWLLEAPEGQRLHLHFEKV
SLAEDDDRLIIRNGDNVEAPPVYDSYEVEYLPIEGLLSSGKHFFVELSTDSSGAAAGMALRYEAFQQGHCYEPFVKY
GNFSSSTPTYPVGTTVEFSCDPGYTLEQGSIIIECVDPHDPQWNETEPACRAVCSGEITDSAGVVLPTEPEPYGRGQ
DSIWGVHVEEDKRIMLDIRVLRIGPGDVLTFYDGDDLTARVLGQYSGPRSHFKLFTSMADVTIQFQSDPGTSVLGYQ
QGFVIHFFEVPRNDTCPELPEIPNGWKSPSQPELVHGTVVTYQCYPGYQVVGSSVLMCQWDLTWSEDLPSCQRVTSC
HDPGDVEHSRRLISSPKFPVGATVQYICDQGFVLMGSSILTCHDRQAGSPKWSDRAPKCLLEQLKPCHGLSAPENGA
RSPEKQLHPAGATIHFSCAPGYVLKGQASIKCVPGHPSHWSDPPPICRAASLDGFYNSRSLDVAKAPAASSTLDAAH
IAAAIFLPLVAMVLLVGGVYFYFSRLQGKSSLQLPRPRPRPYNRITIESAFDNPTYETGSLSFAGDERI

SEQ ID NO. 10

EKALALTGNQGIEAAMDWLMEHEDDPDVDEPLETPLVTYPGEPTSSEQGGLEGSGSAAGEGKPALSEEERQEQTKRM
LELVAQKQREREEREEREALERERQRRRQGQELSAARQRLQEDEMRRAAEERRREKAEELAARQRVREKIERDKAER
AKKYGGSVGSQPPPVAPEPGPVPSSPSQEPPPKREYDQCRIQVRLPDGTSLTQTFRAREQLAAVRLYVELHRGEELG
GGQDPVQLLSGFPRRAFSEADMERPLQELGLVPSAVLIVAKKCPS

SEQ ID NO. 11

SLHLSERADWQYSQRELDAVEVFFSRTARDNRLGCMFVRCAPSSRYTLLFSHGNAVDLGQMCSFYIGLGSRINCNIF
SYDYSGYGVSSGKPSEKNLYADIDAAWQALRTRYGVSPENIILYGQSIGTVPTVDLASRYECAAVILHSPLMSGLRV
AFPDTRKTYCFDAFPSIDKISKVTSPVLVIHGTEDEVIDFSHGLAMYERCPRAVEPLWVEGAGHNDIELYAQYLERL
KQFIHELPNS

SEQ ID NO. 12

MTMEKGMSSGEGLPSRSSQVSAGKITAKELETKQSYKEKRGGFVLVHAGAGYHSESKAKEYKHVCKRACQKAIEKLQ
AGALATDAVTAALVELEDSPFTNAGMGSNLNLLGEIECDASIMDGKSLNFGAVGALSGIKNPVSVANRLLCEGQKGK
LSAGRIPPCFLVGEGAYRWAVDHGIPSCPPNIMTTRFSLAAFKRNKRKLELAERVDTDFMQLKKRRQSSEKENDSGT
LDTVGAVVVDHEGNVAAAVSSGGLALKHPGRVGQAALYGCGCWAENTGAHNPYSTAVSTSGCGEHLVRTILARECSH
ALQAEDAHQALLETMQNKFISSPFLASEDGVLGGVIVLRSCRCSAEPDSSQNKQTLLVEFLWSHTTESMCVGYMSAQ
DGKAKTHISRLPPGAVAGQSVAIEGGVCRLESPVN

SEQ ID NO. 13

KIKDCYGLGSGQNHFIKDSQWEQQAEIFNASYKKYLDREWEEEPLSTATFYFLLPSCLFAMPPEVKGPSGMACVLGI
HWTRSHNFFLYSLNRTLKDKADPEGVWPCAAPIAVSQLSCSSSYLVLACEDGVLTLWDLAKGFPLGVAALPQGCFCQ
SIHFLKYFSVHKGQNMYPEGQVKSQMKCVVLCTDASLHLVEASGTQGPTISVLVERPVKHLDKTICAVAPVPALPGM
VLIFSKNGSVCLMDVAKREIICAFAPPGAFPLEVPWKPVFAVSPDHPCFLLRGDYSHETASTDDAGIQYSVFYFNFE
ACPLLENISKNCTIPQRDLDNMAFPQALPLEKRCERFLQKSYRKLEKNPEKEEEHWARLQRYSLSLQRENFKK

SEQ ID NO. 14

MGKGYYLKGKIGKVPVRFLVDSGAQVSVVHPNLWEEVTDGDLDTLQPFENVVKVANGAEMKILGVWDTAVSLGKLKL
KAQFLVANASAEEAIIGTDLQDHNAILDFEH

SEQ ID NO. 15

MSFICGLQSAARNHVFFRFNSLSNWRKCNTLASTSRGCHQVQVNHIVNKYQGLGVNQCDRWSFLPGNFHFYSTFNNK
RTGGLSSTKSKEIWRITSKCTVWNDAFSRQLLIKEVTAVPSLSVLHPLSPASIRAIRNFHTSPRFQAAPVPLLLMIL
KPVQKLFAIIVGRGIRKWWQALPPNKKEVVKENIRKNKWKLFLGLSSFGLLFVVFYFTHLEVSPITGRSKLLLLGKE
QFRLLSELEYEAWMEEFKNDMLTEKDARYLAVKEVLCHLIECNKDVPGISQINWVIHVVDSPIINAFVLPNGQMFVF
TGFLNSVTDIHQLSFLLGHEIAHAVLGHAAEKAGMVHLLDFLGMIFLTMIWAICPRDSLALLCQWIQSKLQEYMFNR
PYSRKLEAEADKIGLLLAAKACADIRASSVFWQQMEFVDSLHGQPKMPEWLSTHPSHGNRVEYLDRLIPQALKIREM
CNCPPLSNPDPRLLFKLSTKHFLEESEKEDLNITKKQKMDTLPIQKQEQIPLTYIVEKRT

SEQ ID NO. 16

MNNLSFSELCCLFCCPPCPGKIASKLAFLPPDPTYTLMCDESGSRWTLHLSERADWQYSSREKDAIECFMTRTSKGN
RIACMFVRCSPNAKYTLLFSHGNAVDLGQMSSFYIGLGSRINCNIFSYDYSGYGASSGKPTEKNLYADIEAAWLALR
TRYIRPENVIIYGQSIGTVPSVDLAARYESAAVILHSPLTSGMRVAFPDTKKTYCFDAFPNIDKISKITSPVLIIHG
TEDEVIDFSHGLALFERCQRPVEPLWVEGAGHNDVELYGQYLERLKQFVSQELV

SEQ ID NO. 17

GSGCLGAEKREGKNRWQGEASMERLLAQLCGSSAAWPLPLWEGDTTGHCFTQLVLSALPHALLAVLSACYLGTPRSP
DYILPCSPGWRLRLAASFLLSVFPLLDLLPVALPPGAGPGPIGLEVLAGCVAAVAWISHSLALWVLAHSPHGHSRGP
LALALVALLPAPALVLTVLWHCQRGTLLPPLLPGPMARLCLLILQLAALLAYALGWAAPGGPREPWAQEPLLPEDQE
PEVAEDGESWLSRFSYAWLAPLLARGACGELRQPQDICRLPHRLQPTYLARVFQAHWQEGARLWRALYGAFGRCYLA
LGLLKLVGTMLGFSGPLLLSLLVGFLEEGQEPLSHGLLYALGLAGGAVLGAVLQNQYGYEVYKVTLQARGAVLNILY
CKALQLGPSRPPTGEALNLLGTDSERLLNFAGSFHEAWGLPLQLAITLYLLYQQVGVAFVGGLILALLLVPVNKVIA
TRIMASNQEMLQHKDARVKLVTELLSGIRVIKFCGWEQALGARVEACRARELGRLRVIKYLDAACVYLWAALPVVIS
IVIFITYVLMGHQLTATKVFTALALVRMLILPLNNFPWVINGLLEAKVSLDRIQLFLDLPNHNPQAYYSPDPPAEPS
TVLELHGALFSWDPVGTSLETFISHLEVKKGMLVGIVGKVGCGKSSLLAAIAGELHRLRGHVAVRGLSKGFGLATQE
PWIQFATIRDNILFGKTFDAQLYKEVLEACALNDDLSILPAGDQTEVGEKGVTLSGGQRARIALARAVYQEKELYLL
DDPLAAVDADVANHLLHRCILGMLSYTTRLLCTHRTEYLERADAVLLMEAGRLIRAGPPSEILPLVQAVPKAWAENG
QESDSATAQSVQNPEKTKEGLEEEQSTSGRLLQEESKKEGAVALHVYQAYWKAVGQGLALAILFSLLLMQATRNAAD
WWLSHWISQLKAENSSQEAQPSTSPASMGLFSPQLLLFSPGNLYIPVFPLPKAAPNGSSDIRFYLTVYATIAGVNSL
CTLLRAVLFAAGTLQAAATLHRRLLHRVLMAPVTFFNATPTGRILNRFSSDVACADDSLPFILNILLANAAGLLGLL
AVLGSGLPWLLLLLPPLSIMYYHVQRHYRASSRELRRLGSLTLSPLYSHLADTLAGLSVLRATGATYRFEEENLRLL
ELNQRCQFATSATMQWLDIRLQLMGAAVVSAIAGIALVQHQQGLANPGLVGLSLSYALSLTGLLSGLVSSFTQTEAM
LVSVERLEEYTCDLPQEPQGQPLQLGTGWLTQGGVEFQDVVLAYRPGLPNALDGVTFCVQPGEKLGIVGRTGSGKSS
LLLVLFRLLEPSSGRVLLDGVDTSQLELAQLRSQLAIIPQEPFLFSGTVRENLDPQGLHKDRALWQALKQCHLSEVI
TSMGGLDGELGEGGRSLSLGQRQLLCLARALLTDAKILCIDEATASVDQKTDQLLQQTICKRFANKTVLTIAHRLNT
ILNSDRVLVLQAGRVVELDSPATLRNQPHSLFQQLLQSSQQGVPASLGGP

SEQ ID NO. 18

MAAVRVLVASRLAAASAFTSLSPGGRTPSQRAALHLSVPRPAARVALVLSGCGVYDGTEIHEASAILVHLSRGGAEV
QIFAPDVPQMHVIDHTKGQPSEGESRNVLTESARIARGKITDLANLSAANHDAAIFPGGFGAAKNLSTFAVDGKDCK
VNKEVERVLKEFHQAGKPIGLCCIAPVLAAKVLRGVEVTVGHEQEEGGKWPYAGTAEAIKALGAKHCVKEVSLRSVL
GGFFRNSAHEAHVDQKNKVVTTPAFMCETALHYIHDGIGAMVRKVLELTGK

SEQ ID NO. 19

MAELTALESLIEMGFPRGRAEKALALTGNQGIEAAMDWLMEHEDDPDVDEPLETPLGHILGREPTSSEQGGLEGSGS
AAGEGKPALSEEERQEQTKRMLELVAQKQREREEREEREALERERQRRRQGQELSAARQRLQEDEMRRAAEERRREK
AEELAARQRVREKIERDKAERAKKYGGSVGSQPPPVAPEPGPVPSSPSQEPPTKREYDQCRIQVRLPDGTSLTQTFR
AREQLAAVRLYVELHRGEELGGGQDPVQLLSGFPRRAFSEADMERPLQELGLVPSAVLIVAKKCPS

SEQ ID NO. 20

QVKLKIPFGNKLLDAVCLVPNKSLTYGIILTHGASGDMNLPHLMSLASHLASHGFFCLRFTCKGLNIVHRIKAYKSV
LNYLKTSGEYKLAGVFLGGRSMGSRAAASVMCHIEPDDGDDFVRGLICISYPLHHPKQQHKLRDEDLFRLKEPVLFV
SGSADEMCEKNLLEKVAQKMQAPHKIHWIEKANHSMAVKGRSTNDVFKEINTQILFWIQEITEMDKK

SEQ ID NO. 21

MNGLSLSELCCLFCCPPCPGRIAAKLAFLPPEATYSLVTEPEPGPGGAGAAPLGTLRASSGAPGRWKLHLTERADFQ
YSQRELDTIEVFPTKSARRNRVSCMYVRCVTGARYTVLFSHSNAVDLGQMSSFYIGLGSRLHCNIPSYDYSGYGASS
GRPSERNLYADIDAAWQALRTRYGISPDSIILYGQSIGTVPTVDLASRYECAAVVLHSPLTSGMRVAFPDTKKTYCF
DAFPNIEKVSKITSPVLIIHGTEDEVIDFSHGLALYERCPKAVEPLWVEGTRHNDIELYSQYLEGLRRFISQEL

SEQ ID NO. 22

AQGKDQMWYEDALASSHPIILYLHGNAGTRCLFFTLQVLSSLGYHVVTFDYRGWGDSVGTPSERGMTYDALHVFDWI
KARSGDNPVYIWGHSLGTGVATNLVRRLCERETPPDALILESPFTNIREEAKSHPFSIYRYFPGFDWFFLDPITSSG
IKFANDENVKHISCPLLILHAEDDPVVPFQLGRKLYSIAAPARSFRDFKVQFVPFHSDLGYRHKYIYKSPELPRILR
EFLGKSEPEHQH

SEQ ID NO. 23

MDASIMDGKDLSAGAVSAVQCIANPIKLARLVMEKTPHCFLTDQGAAQFAAAMGVPEIPGEKLVTERNKKRLEKEKH
EKGAQKTDCQKNLGTVGAVALDCKGNVAYATSTGGIVNKMVGRVGDSPCLAGAGGYADNDIGAVSTTGHGESILKVN
LARLTLFHIEQGKTVEEAADLSLGYMKSRVKGLGGLIVVSKTGDWVAKWTSTSMPWAAAKDGKLHFGIDPDDTTITD
LP

SEQ ID NO. 24

MLRGVLGKTFRLVGYTIQYGCIAHCAFEYVGGVVMCSGPSMEPTIQNSDIVFAENLSRHFYGIQRGDIVIAKSPSDP
KSNICKRVIGLEGDKILTTSPSDFFKSHSYVPMGHVWLEGDNLQNSTDSRCYGPIPYGLIRGRIFFKIWPLSDFGFL
RASPNGHRFSDD

SEQ ID NO. 25

MMDSPKIGNGLPVIGPGTDIGISSLHMVGYLGKNFDSAKVPSDEYCPACREKGKLKALKTYRISFQESIFLCEDLQC
IYPLGSKSLNNLISPDLEECHTPHKPQKRKSLESSYKDSLLLANSKKTRNYIAIDGGKVLNSKHNGEVYDETSSNLP
DSSGQQNPIRTADSLERNEILEADTVDMATTKDPATVDVSGTGRPSPQNEGCTSKLEMPLESKCTSFPQALCVQWKN
AYALCWLDCILSALVHSEELKNTVTGLCSKEESIFWRLLTKYNQANTLLYTSQLSGVKDGDCKKLTSEIFAEIETCL
NEVRDEIFISLQPQLRCTLGDMESPVFAFPLLLKLETHIEKLFLYSFSWDFECSQCGHQYQNRHMKSLVTFTNVIPE
WHPLNAAHFGPCNNCNSKSQIRKMVLEKVSPIFMLHFVEGLPQNDLQHYAFHFEGCLYQITSVIQYRANNHFITWIL
DADGNWLECDDLKGPCSERRKKFEVPASEIHIVIWERKISQVTDKEAACLPLKKTNDQHALSNEKPVSLTSCSVGDA
ASAETASVTHPKDISVAPRTLSQDTAVTHGDHLLSGPKGLVDNILPLTLEETIQKTASVSQLNSEAFLLENKPVAEN
TGILKTNTLLSQESLMASSVSAPCNEKLIQDQFVDISFPSQVVNTNMQSVQLNTEDTVNTKSVNNTDATGLIQGVKS
VEIEKDAQLKQFLTPKTEQLKPERVTSQVSNLKKKETTADSQTTTSKSLQNQSLKENQKKPFVGSWVKGLISRGASF
MPLCVSAHNRNTITDLQPSVKGVNNFGGFKTKGINQKASHVSKKARKSASKPPPISKPPAGPPSSNGTAAHPHAHAA
SEVLEKSGSTSCGAQLNHSSYGNGISSANHEDLVEGQIHKLRLKLRKKLKAEKKKLAALMSSPQSRTVRSENLEQVP
QDGSPNDCESIEDLNELPYPIDIASESACTTVPGVSLYSSQTHEEILAELLSPTPVSTELSENGEGDFRYLGMGDSH
IPPPVPSEFNDVSQNTHLRQDHNYCSPTKKNPCEVQPDSLTNNACVRTLNLESPMKTDIFDEFFSSSALNALANDTL
DLPHFDEYLFENY

SEQ ID NO. 26

MLTGVTDGIFCCLLGTPPNAVGPLESVESSDGYTFVEVKPGRVLRVKHAGPAPAAAPPPPSSASSDAAQGDLSGLVR
CQRRITVYRNGRLLVENLGRAPRADLLHGQNGSGEPPAALEVELADPAGSDGRLAPGSAGSGSGSGSGGRRRRARRP
KRTIHIDCEKRITSCKGAQADVVLFFIHGVGGSLAIWKEQLDFFVRLGYEVVAPDLAGHGASSAPQVAAAYTFYALA
EDMRAIFKRYAKKRNVLIGHSYGVSFCTFLAHEYPDLVHKVIMINGGGPTALEPSFCSIFNMPTCVLHCLSPCLAWS
FLKAGFARQGAKEKQLLKEGNAFNVSSFVLRAMMSGQYWPEGDEVYHAELTVPVLLVHGMHDKFVPVEEDQRMAEIL
LLAFLKLIDEGSHMVMLECPETVNTLLHEFLLWEPEPSPKALPEPLPAPPEDKK

SEQ ID NO. 27

MRRQWGSANRAAEQAGCMVSASRAGQPEAGPWSCSGVILSRSPGLVLCHGGIFVPFLRAGSEVLTAGAVFLPGDSCR
DDLRLHVQWAPTGAPRGQARTGPPRLARPMRDLSPPPPSQASLSQSCDWRITETLGWFALLGVRLGQEEWRRRGPMA
VSPLGAVPKGAPLLVCGSPFGAFCPDIFLNTLSCGVLSNVAGPLLLTDARCLPGTEGGGVFTARPAGALVALVVAPL
CWKAGEWVGFTLLCAAAPLFRAARDALHRLPHSTAALAALLPPEVGVPWGLPLRDSGPLWAAAAVLVECGTVWGSGV
AVAPRLVVTCRHVSPREAARVLVRSTTPKSVAIWGRVVFATQETCPYDIAVVSLEEDLDDVPIPVPAEHFHEGEAVS
VVGFGVFGQSCGPSVTSGILSAVVQVNGTPVMLQTTCAVHSGSSGGPLFSNHSGNLLGIITSNTRDNNTGATYPHLN
FSIPITVLQPALQQYSQTQDLGGLRELDRAAEPVRVVWRLQRPLAEAPRSKL

SEQ ID NO. 28

MAVARLAAVAAWVPCRSWGWAAVPFGPHRGLSVLLARIPQRAPRWLPACRQKTSLSFLNRPDLPNLAYKKLKGKSPG
IIFIPGYLSYMNGTKALAIEEFCKSLGHACIRFDYSGVGSSDGNSEESTLGKWRKDVLSIIDDLADGPQILVGSSLG
GWLMLHAAIARPEKVVALIGVATADTLVTKFNQLPVELKKEVEMKGVWSMPSKYSEEGVYNVQYSFIKEAEHHCLLH
SPIPVNCPIRLLHGMKDDIVPWHTSMQVADRVLSTDVDVILRKHSDHRMREKADIQLLVYTIDDLIDKLSTIVN

SEQ ID NO. 29

QQGSITLSLWTLPDVLIIHLKRFRQEGDRRMKLQNMVKFPLTGLDMTPHVVKRSQSSWSLPSHWSPWRRPYGLGRDP
EDYIYDLYAVCNHHGTMQGGHYTAYCKNSVDGLWYCFDDSDVQQLSEDEVCTQTAYILFYQRRTAIPSWSANSSVAG
STSSSLCEHWVSRLPGSKPASVTSAASSRRTSLASLSESVEMTGERSEDDGGCFSTRPFVRSVQRQSLSSRSSVTSP
LAVNENCMRPSWSLSAKLQMRSNSPSRFSGDSPIHSSASTLEKIGEAADDKVSISCFGSLRNLSSSYQEPSDSHSLR
EHKAVGRAPLAVMEGVFKDESDTRRLNSSVVDTQSKHSAQGDRLPPLSGPFDNNNQIAYVDQSDSVDSSPVKEVKAP
SHPGSLAKKPESTTKRSPSSKGTSEPEKSLRKGRPALASQESSLSSTSPSSPLPVKVSLKPSRSRSKADSSSRGSGR
HSSPAPAQPKKESSPKSQDSVSSPSPQKQKSASALTYTASSTSAKKASGPATRSPFPPGKSRTSDHSLSREGSRQSL
GSDRASATSTSKPNSPRVSQAPAGEGRGAGKHVRSSSMASLRSPSTSIKSGLKRDSKSEDKGLSFFKSALRQKETRR
STDLGKTALLSKKAGGSSVKSVCKNTGDDEAERGHQPPASQQPNANTTGKEQLVTKDPASAKHSLLSARKSKSSQLD
SGVPSSPGGRQSAEKSSKKLSSSMQTSARPSQKPQ

SEQ ID NO. 30

MCGIWALFGSDDCLSVQCLSAMKIAHRGPDAFRFENVNGYTNCCFGFHRLAVVDPLFGMQPIRVKKYPYLWLCYNGE
IYNHKKMQQHFEFEYQTKVDGEIILHLYDKGGIEQTICMLDGVFAFVLLDTANKKVFLGRDTYGVRPLFKAMTEDGF
LAVCSEAKGLVTLKHSATPFLKVEPFLPGHYEVLDLKPNGKVASVEMVKYHHCRDVPLHALYDNVEKLFPGFEIETV
KNNLRILFNNAVKKRLMTDRRIGCLLSGGLDSSLVAATLLKQLKEAQVQYPLQTFAIGMEDSPDLLAARKVADHIGS
EHYEVLFNSEEGIQALDEVIFSLETYDITTVRASVGMYLISKYIRKNTDSVVIFSGEGSDELTQGYIYFHKAPSPEK
AEEESERLLRELYLFDVLRADRTTAAHGLELRVPFLDHRFSSYYLSLPPEMRIPKNGIEKHLLRETFEDSNLIPKEI
LWRPKEAFSDGITSVKNSWFKILQEYVEHQVDDANMANAAQKFPFNTPKTKEGYYYRQVFERHYPGRADWLSHYWMP
KWINATDPSARTLTHYKSAVKA

SEQ ID NO. 31

QANCQIAILYQRFQRVVFGISQLLCFSALISELTNQKEVAAWTYHYSTKAYSWNISRKYCQNRYTDLVAIQNKNEID
YLNKVLPYYSSYYWIGIRKNNKTWTWVGTKKALTNEAENWADNEPNNKRNNEDCVEIYIKSPSAPGKWNDEHCLKKK
HALCYTASCQDMSCSKQGECLETIGNYTCSCYPGFYGPECEYVRECGELELPQHVLMNCSHPLGNFSFNSQCSFHCT
DGYQVNGPSKLECLASGIWTNKPPQCLAAQCPPLKIPERGNNTCLHSAKAFQHQSSCSFSCEEGFALVGPEVVQCTA
SGVWTAPAPVCKAVQCQHLEAPSEGTMDCVHPLTAFAYGSSCKFECQPGYRVRGLDMLRCIDSGHWSAPLPTCEAIS
CEPLESPVHGSMDCSPSLRAFQYDTNCSFRCAEGFMLRGADIVRCDNLGQWTAPAPVCQALQCQDLPVPNEARVNCS
HPFGAFRYQSVCSFTCNEGLLLVGASVLQCLATGNWNSVPPECQAIPCTPLLSPQNGTMTCVQPLGSSSYKSTCQFI
CDEGYSLSGPERLDCTRSGRWTDSPPMCEAIKCPELFAPEQGSLDCSDTRGEFNVGSTCHFSCDNGFKLEGPNNVEC
TTSGRWSATPPTCKGIASLPTPGVQCPALTTPGQGTMYCRHHPGTFGFNTTCYFGCNAGFTLIGDSTLSCRPSGQWT
AVTPACRAVKCSELHVNKPIANNCSNLWGNFSYGSICSFHCLEGQLLNGSAQTACQENGHWSTTVPTCQAGPLTIQE
ALTYFGGAVASTIGLIMGGTLLALLRKRFRQKDDGKCPLNPHSHLGTYGVFTNAAFDPSP

SEQ ID NO. 32

MLRGPGPGLLLLAVQCLGTAVPSTGASKSKRQAQQMVQPQSPVAVSQSKPGCYDNGKHYQINQQWERTYLGNALVCT
CYGGSRGFNCESKPEAEETCFDKYTGNTYRVGDTYERPKDSMIWDCTCIGAGRGRISCTIANRCHEGGQSYKIGDTW
RRPHETGGYMLECVCLGNGKGEWTCKPIAEKCFDHAAGTSYVVGETWEKPYQGWMMVDCTCLGEGSGRITCTSRNRC
NDQDTRTSYRIGDTWSKKDNRGNLLQCICTGNGRGEWKCERHTSVQTTSSGSGPFTDVRAAVYQPQPHPQPPPYGHC
VTDSGVVYSVGMQWLKTQGNKQMLCTCLGNGVSCQETAVTQTYGGNSNGEPCVLPFTYNGRTFYSCTTEGRQDGHLW
CSTTSNYEQDQKYSFCTDHTVLVQTRGGNSNGALCHFPFLYNNHNYTDCTSEGRRDNMKWCGTTQNYDADQKFGFCP
MAAHEEICTTNEGVMYRIGDQWDKQHDMGHMMRCTCVGNGRGEWTCIAYSQLRDQCIVDDITYNVNDTFHKRHEEGH
MLNCTCFGQGRGRWKCDPVDQCQDSETGTFYQIGDSWEKYVHGVRYQCYCYGRGIGEWHCQPLQTYPSSSGPVEVFI
TETPSQPNSHPIQWNAPQPSHISKYILRWRPKNSVGRWKEATIPGHLNSYTIKGLKPGVVYEGQLISIQQYGHQEVT
RFDFTTTSTSTPVTSNTVTGETTPFSPLVATSESVTEITASSFVVSWVSASDTVSGFRVEYELSEEGDEPQYLDLPS
TATSVNIPDLLPGRKYIVNVYQISEDGEQSLILSTSQTTAPDAPPDTTVDQVDDTSIVVRWSRPQAPITGYRIVYSP
SVEGSSTELNLPETANSVTLSDLQPGVQYNITIYAVEENQESTPVVIQQETTGTPRSDTVPSPRDLQFVEVTDVKVT
IMWTPPESAVTGYRVDVIPVNLPGEHGQRLPISRNTFAEVTGLSPGVTYYFKVFAVSHGRESKPLTAQQTTKLDAPT
NLQFVNETDSTVLVRWTPPRAQITGYRLTVGLTRRGQPRQYNVGPSVSKYPLRNLQPASEYTVSLVAIKGNQESPKA
TGVFTTLQPGSSIPPYNTEVTETTIVITWTPAPRIGFKLGVRPSQGGEAPREVTSDSGSIVVSGLTPGVEYVYTIQV
LRDGQERDAPIVNKVVTPLSPPTNLHLEANPDTGVLTVSWERSTTPDITGYRITTTPTNGQQGNSLEEVVRADQSSC
TFDNLSPGLEYNVSVYTVKDDKESVPISDTIIPAVPPPTDLRFTNIGPDTMRVTWAPPPSIDLTNFLVRYSPVKNEE
DVAELSISPSDNAVVLTNLLPGTEYVVSVSSVYEQHESTPLRGRQKTGLDSPTGIDFSDITANSFTVHWIAPRATIT
GYRIRHHPEHFSGRPREDRVPHSRNSITLTNLTPGTEYVVSIVALNGREESPLLIGQQSTVSDVPRDLEVVAATPTS
LLISWDAPAVTVRYYRITYGETGGNSPVQEFTVPGSKSTATISGLKPGVDYTITVYAVTGRGDSPASSKPISINYRT
EIDKPSQMQVTDVQDNSISVKWLPSSSPVTGYRVTTTPKNGPGPTKTKTAGPDQTEMTIEGLQPTVEYVVSVYAQNP
SGESQPLVQTAVTNIDRPKGLAFTDVDVDSIKIAWESPQGQVSRYRVTYSSPEDGIHELFPAPDGEEDTAELQGLRP
GSEYTVSVVALHDDMESQPLIGTQSTAIPAPTDLKFTQVTPTSLSAQWTPPNVQLTGYRVRVTPKEKTGPMKEINLA
PDSSSVVVSGLMVATKYEVSVYALKDTLTSRPAQGVVTTLENVSPPRRARVTDATETTITISWRTKTETITGFQVDA
VPANGQTPIQRTIKPDVRSYTITGLQPGTDYKIYLYTLNDNARSSPVVIDASTAIDAPSNLRFLATTPNSLLVSWQP
PRARITGYIIKYEKPGSPPREVVPRPRPGVTEATITGLEPGTEYTIYVIALKNNQKSEPLIGRKKTDELPQLVTLPH
PNLHGPEILDVPSTVQKTPFVTHPGYDTGNGIQLPGTSGQQPSVGQQMIFEEHGFRRTTPPTTATPIRHRPRPYPPN
VGEEIQIGHIPREDVDYHLYPHGPGLNPNASTGQEALSQTTISWAPFQDTSEYIISCHPVGTDEEPLQFRVPGTSTS
ATLTGLTRGATYNVIVEALKDQQRHKVREEVVTVGNSVNEGLNQPTDDSCFDPYTVSHYAVGDEWERMSESGFKLLC
QCLGFGSGHFRCDSSRWCHDNGVNYKIGEKWDRQGENGQMMSCTCLGNGKGEFKCDPHEATCYDDGKTYHVGEQWQK
EYLGAICSCTCFGGQRGWRCDNCRRPGGEPSPEGTTGQSYNQYSQRYHQRTNTNVNCPIECFMPLDVQADREDSRE

SEQ ID NO. 33

RYNKNLEEAKRIGIKKAITANISIGAAFLLIYASYALAFWYGTTLVLSGEYSIGQVLTVFFSVLIGAFSVGQASPSI
EAFANARGAAYEIFKIIDNKPSIDSYSKSGHKPDNIKGNLEFRNVHFSYPSRKEVKILKGLNLKVQSGQTVALVGNS
GCGKSTTVQLMQRLYDPTEGMVSVDGQDIRTINVRFLREIIGVVSQEPVLFATTIAENIRYGRENVTMDEIEKAVKE
ANAYDFIMKLPHKFDTLVGERGAQLSGGQKQRIAIARALVRNPKILLLDEATSALDTESEAVVQVALDKARKGRTTI
VIAHRLSTVRNADVIAGFDDGVIVEKGNHDELMKEKGIYFKLVTMQDESIPPVSFWRIMKLNLTEWPYFVVGVFCAI
INGGLQPAFAIIFSKIIGVFTRIDDPETKRQNSNLFSLLFLALGIISFITFFLQGFTFGKAGEILTKRLRYMVFRSM
LRQDVSWFDDPKNTTGALTTRLANDAAQVKGAIGSRLAVITQNIANLGTGIIISFIYGWQLTLLLLAIVPIIAIAGV
VEMKMLSGQALKDKKELEGSGKIATEAIENFRTVVSLTQEQKFEHMYAQSLQVPYRNSLRKAHIFGITFSFTQAMMY
FSYAGCFRFGAYLVAHKLMSFEDVLLVFSAVVFGAMAVGQVSSFAPDYAKAKISAAHIIMIIEKTPLIDSYSTEGLM
PNTLEGNVTFGEVVFNYPTRPDIPVLQGLSLEVKKGQTLALVGSSGCGKSTVVQLLERFYDPLAGKVLICELFQLLD
GKEIKRLNVQWLRAHLGIVSQEPILFDCSIAENIAYGDNSRVVSQEEIVRAAKEANIHAFIESLPNKYSTKVGDKGT
QLSGGQKQRIAIARALVRQPHILLLDEATSALDTESEKVVQEALDKAREGRTCIVIAHRLSTIQNADLIVVFQNGRV
KEHGTHQQLLAQKGIYFSMVSVQAGTKRQ

SEQ ID NO. 34

MSLSFCGNNISSYNINDGVLQNSCFVDALNLVPHVFLLFITFPILFIGWGSQSSKVQIHHNTWLHFPGHNLRWILTF
ALLFVHVCEIAEGIVSDSRRESRHLHLFMPAVMGFVATTTSIVYYHNIETSNFPKLLLALFLYWVMAFITKTIKLVK
YCQSGLDISNLRFCITGMMVILNGLLMAVEINVIRVRRYVFFMNPQKVKPPEDLQDLGVRFLQPFVNLLSKATYWWM
NTLIISAHKKPIDLKAIGKLPIAMRAVTNYVCLKDAYEEQKKKVADHPNRTPSIWLAMYRAFGRPILLSSTFRYLAD
LLGFAGPLCISGIVQRVNETQNGTNNTTGISETLSSKEFLENAYVLAVLLFLALILQRTFLQASYYVTIETGINLRG
ALLAMIYNKILRLSTSNLSMGEMTLGQINNLVAIETNQLMWFLFLCPNLWAMPVQIIMGVILLYNLLGSSALVGAAV
IVLLAPIQYFIATKLAEAQKSTLDYSTERLKKTNEILKGIKLLKLYAWEHIFCKSVEETRMKELSSLKTFALYTSLS
SKLLTPFFAQTFVTHAYASGNNLKPAEAFASLSLFHILVTPLFLLSTVVRFAVKAIISVQKLNEFLLSDEIGDDSWR
TGESSLPFESCKKHTGVQPKTINRKQPGRYRLDSYEQSTRRLRPAETEDIAIKVTNGYFSWGSGLATLSNIDIRIPT
GQLTMIVGQVGCGKSSLLLAILGEMQTLEGKVHWSKYVYFYRNRYSVAYAAQKPWLLNATVEENITFGSPFNKQRYK
AVTDACSLQPDIDLLPFGDQTEIGERGINLSGGQRQRICVARALYQNTNIVFLDDPFSALDIHLSDELMQEGILKFL
QDDKRTLVLVTHKLQYLTHADWIIAMKDGSVLREGTLKDIQTKDVELYEHWKTLMNRQDQELEKDMEADQTTLERKT
LRRAMYSREAKAQMEDEDEEEEEEEDEDDNNSTVMRLRTKMPWKTCWRYLTSGGFFLLILMIFSKLLKHSVIVAIDY
WLATWTSEYSINNTGKADQTYYVAGFSILCGAGIFLCLVTSLTVEWMGLTAAKNLHHNLLNKIILGPIRFFDTTPLG
LILNRFSADTNIIDQHIPPTLESLTRSTLLCLSAIGMISYATPVFLVALLPLGVAFYFIQKYFRVASKDLQELDDST
QLPLLCHFSETAEGLTTIRAFRHETRFKQRMLELTDTNNIAYLFLSAANRWLEVRTDYLGACIVLTASIASISGSSN
SGLVGLGLLYALTITNYLNWVVRNLADLEVQMGAVKKVNSFLTMESENYEGTMDPSQVPEHWPQEGEIKIHDLCVRY
ENNLKPVLKHVKAYIKPGQKVGICGRTGSGKSSLSLAFFRMVDIFDGKIVIDGIDISKLPLHTLRSRLSIILQDPIL
FSGSIRFNLDPECKCTDDRLWEALEIAQLKNMVKSLPGGLDAVVTEGGENFSVGQRQLFCLARAFVRKSSILIMDEA
TASIDMATENILQKVVMTAFADRTVVTIAHRVHTILTADLVIVMKRGNILEYDTPESLLAQENGVFASFVRADM

SEQ ID NO. 35

RAELVALTAVQSEQGEAGGGGSPRRLGLLGSPLPPGAPLPGPGSGSGSACGQRSSAAHKRYRRLQNWGYNVLERPRG
WAFVYHVFIFLLVFSCLVLSVLSTIQEHQELANECLLILEFVMIVVFGLEYIVRVWSAGCCCRYRGWQGRFRFARKP
FCVIDFIVFVASVAVIAAGTQGNIFATSALRSMRFLQILRMVRMDRRGGTWKLLGSVVYAHSKELITAWYIGFLVLI
FASFLVYLAEKDANSDFSSYADSLWWGTITLTTIGYGDKTPHTWLGRVLAAGFALLGISFFALPAGILGSGFALKVQ
EQHRQKHFEKRRNPAANLIQAAWRLYSTDMSPAYLTATWYYYDSILPSFRELALLFEHVQRARNGGLRPLEVRPAPV
PDGAPSRYPPVATCHRPGSTSFCPGESSRMGIKDRIRMGSSQRRTGPSKQHLAPPTMPTSPSSEQVGEATSPTKVQK
SWSFNDRTRFRASLRLKPRTSAEDAPSEEVAEEKSYQCELTVDDIMPAVKTVIRSIRILKFLVAKRKFKETLRPYDV
KDVIEQYSAGHLDMLGRIKSLQTRVDQIVGRGPGDRKAREKGDKGPSDAEVVDEISMMGRVVKVEKQVQSIERKLDL
LVGFYSRWLRSGTSASLGAVQVPLFDPDITSDYHSPVDHEDISVSAQTLSISRSVSTNMD

SEQ ID NO. 36

QIFPWKCQSTQRDLWNIFKLWGWTMLCCDFLAHHGTDCWTYHYSEKPMNWQRARRFCRDNYTDLVAIQNKAEIEYLE
KTLPFSRSYYWIGIRKIGGIWTWVGTNKSLTEEAENWGDGEPNNKKNKEDCVEIYIKRNKDAGKWNDDACHKLKAAL
CYTASCQPWSCSGHGECVEIINNYTCNCDVGYYGPQCQFVIQCEPLEAPELGTMDCTHPLGNFSFSSQCAFSCSEGT
NLTGIEETTCGPFGNWSSPEPTCQVIQCEPLSAPDLGIMNCSHPLASFSFTSACTFICSEGTELIGKKKTICESSGI
WSNPSPICQKLDKSFSMIKEGDYNPLFIPVAVMVTAFSGLAFIIWLARRLKKG

SEQ ID NO. 37

QKEGKKERAVVDKVFFSRLIQILKIMVPRTFCKETGYLVLIAVMLVSRTYCDVWMIQNGTLIESGIIGRSRKDFKRY
LLNFIAAMPLISLVNNFLKYGLNELKLCFRVRLTKYLYEEYLQAFTYYKMGNLDNRIANPDQLLTQDVEKFCNSVVD
LYSNLSKPFLDIVLYIFKLTSAIGAQGPASMMAYLVVSGLFLTRLRRPIGKMTITEQKYEGEYRYVNSRLITNSEEI
AFYNGNKREKQTVHSVFRKLVEHLHNFILFRFSMGFIDSIIAKYLATVVGYLVVSRPFLDLSHPRHLKSTHSELLED
YYQSGRNLLRMSQALGRIVLAGREMTRLAGFTARITELMQVLKDLNHGKYERTMVSQQEKGIEGVQVIPLIPGAGEI
IIADNIIKFDHVPLATPNGDVLIRDLNFEVRSGANVLICGPNGCGKSSLFRVLGELWPLFGGRLTKPERGKLFYVPQ
RPYMTLGTLRDQVIYPDGREDQKRKGISDLVLKEYLDNVQLGHILEREGGWDSVQDWMDVLSGGEKQRMAMARLFYH
KPQFAILDECTSAVSVDVEGYIYSHCRKVGITLFTVSHRKSLWKHHEYYLHMDGRGNYEFKQITEDTVEFGS

SEQ ID NO. 38

MGHLLTLVFILALAGPVLGLKECTRGSAVWCQNVKTASDCGAVKHCLQTVWNKPTVKSLPCDICKDVVTAAGDMLKD
NATEEEILVYLEKTCDWLPKPNMSASCKEIVDSYLPVILDIIKGEMSRPGEVCSALNLCESLQKHLAELNHQKQLES
NKIPELDMTEVVAPFMANIPLLLYPQDGPRSKPQPKDNGDVCQDCIQMVTDIQTAVRTNSTFVQALVEHVKEECDRL
GPGMADICKNYISQYSEIAIQMMMHMQPKEICALVGFCDEVKEMPMQTLVPAKVASKNVIPALELVEPIKKHEVPAK
SDVYCEVCEFLVKEVTKLIDNNKTEKEILDAFDKMCSKLPKSLSEECQEVVDTYGSSILSILLEEVSPELVCSMLHL
CSGTRLPALTVHVTQPKDGGFCEVCKKLVGYLDRNLEKNSTKQEILAAIEKGCSFLPDPYQKQCDQFVAEYEPVLIE
ILVEVMDPSFVCLKIGACPSAHKPLLGTEKCIWGPSYWCQNTETAAQCNAVEHCKRHVWN

SEQ ID NO. 39

LTERADFQYSQRELDTIEVFPTKSARGNRVSCMYVRCVPGARYTVFFSHGNAVDLSQMSSFYIGLGSRLHCNIFYDY
SGYGASAGRPSERNLYADIDAAWQALHTRYGISPDSIILYGQSIGTVPTVDLASRYECAAVVLHSPLTSGMRVAFPD
TKTYCFDAFPNIEKVSKITSPVLIIHGMEDEVIDFSHGLALYERCPKAVEPLWVEGAGHNDIELYSQYLERLRRFIS
QELPS

SEQ ID NO. 40

MSEPGGGGGEDGSAGLEVSAVQNVADVSVLQKHLRKLVPLLLEDGGEAPAALEAALEEKSALEQMRKFLSDPQVHTV
LVERSTLKEDVGDEGEEEKEFISYNINIDIHYGVKSNSLAFIKRTPVIDADKPVSSQLRVLTLSEDSPYETLHSFIS
NAVAPFFKSYIRESGKADRDGDKMAPSVEKKIAELEMGLLHLQQNIEIPEISLPIHPMITNVAKQCYERGEKPKVTD
FGDKVEDPTFLNQLQSGVNRWIREIQKVTKLDRDPASGTALQEISFWLNLERALYRIQEKRESPEVLLTLDILKHGK
RFHATVSFDTDTGLKQALETVNDYNPLMKDFPLNDLLSATELDKIRQALVAIFTHLRKIRNTKYPIQRALRLVEAIS
RDLSSQLLKVLGTRKLMHVAYEEFEKVMVACFEVFQTWDDEYEKLQVLLRDIVKRKREENLKMVWRINPAHRKLQAR
LDQMRKFRRQHEQLRAVIVRVLRPQVTAVAQQNQGEVPEPQDMKVAEVLFDAADANAIEEVNLAYENVKEVDGLDVS
KEGTEAWEAANKRYDERIDRVETRITARLRDQLGTAKNANEMFRIFSRFNALFVRPHIRGAIREYQTQLIQRVKDDI
ESLHDKFKVQYPQSQACKMSHVRDLPPVSGSIIWAKQIDRQLTAYMKRVEDVLGKGWENHVEGQKLKQDGDSFRMKL
NTQEIFDDWARKVQQRNLGVSGRIFTIESTRVRGRTGNVLKLKVNFLPEIITLSKEVRNLKWLGFRVPLAIVNKAHQ
ANQLYPFAISLIESVRTYERTCEKVEERNTISLLVAGLKKEVQALIAEGIALVWESYKLDPYVQRLAETVFNFQEKV
DDLLIIEEKIDLEVRSLETCMYDHKTFSEILNRVQKAVDDLNLHSYSNLPIWVNKLDMEIERILGVRLQAGLRAWTQ
VLLGQAEDKAEVDMDTDAPQVSHKPGGEPKIKNVVHELRITNQVIYLNPPIEECRYKLYQEMFAWKMVVLSLPRIQS
QRYQVGVHYELTEEEKFYRNALTRMPDGPVALEESYSAVMGIVSEVEQYVKVWLQYQCLWDMQAENIYNRLGEDLNK
WQALLVQIRKARGTFDNAETKKEFGPVVIDYGKVQSKVNLKYDSWHKEVLSKFGQMLGSNMTEFHSQISKSRQELEQ
HSVDTASTSDAVTFITYVQSLKRKIKQFEKQVELYRNGQRLLEKQRFQFPPSWLYIDNIEGEWGAFNDIMRRKDSAI
QQQVANLQMKIVQEDRAVESRTTDLLTDWEKTKPVTGNLRPEEALQALTIYEGKFGRLKDDREKCAKAKEALELTDT
GLLSGSEERVQVALEELQDLKGVWSELSKVWEQIDQMKEQPWVSVQPRKLRQNLDALLNQLKSFPARLRQYASYEFV
QRLLKGYMKINMLVIELKSEALKDRHWKQLMKRLHVNWVVSELTLGQIWDVDLQKNEAIVKDVLLVAQGEMALEEFL
KQAKVWNTYELDLVNYQNKCRLIRGWDDLFNKVKEHINSVSANKLSPYYKVFEEDALSWEDKLNRIMALFDVWIDVQ
RRWVYLEGIFTGSADIKHLLPVETQRFQSISTEFLALMKKVSKSPLVMDVLNIQGVQRSLERLADLLGKIQKALGEY
LERERSSFPRFYFVGDEDLLEIIGNSKNVAKLQKHFKKMFAGVSSIILNEDNSVVLGISSREGEEVMFKTPVSITEH
PKINEWLTLVEKEMRVTLAKLLAESVTEVEIFGKATSIDPNTYITWIDKYQAQLVVLSAQIAWSENVETALSSMGGG
GDAAPLHSVLSNVEVTLNVLADSVLMEQPPLRRRKLEHLITELVHQRDVTRSLIKSKIDNAKSFEWLSQMRFYFDPK
QTDVLQQLSIQMANAKFNYGFEYLGVQDKLVQTPLTDRCYLTMTQALEARLGGSPFGPAGTGKTESVKALGHQLGRF
VLVFNCDETFDFQANGRIFVGLCQVGAWGCFDEFNRLEERMLSAVSQQVQCIQEALREHSNPNYDKTSAPITCELLN
KQVKVSPDMAIFITMNPAYAGRSNLPDNLKKLFRSLAMTKPDRQLIAQVMLYSQGFRTAEVLANKIVPFFKLCDEQL
SSQSHYDFGLRALKSVLVSAGNVKRERIQKIKREKEERGEAVDEGEIAENLPEQEILIQSVCETMVPKLVAEDIPLL
FSLLSDVFPGVQYHRGEMTALREELKKVCQEMYLTYGDGEEVGGMWVEKVLQLYQITQINHGLMMVGPSGSGKSMAW
RVLLKALERLEGVEGVAHIIDPKAISKDHLYGTLDPNTREWTDGLFTHVLRKIIDSVRGELQKRQWIVFDGDVDPEW
VENLNSVLDDNKLLTLPNGERLSLPPNVRIMFEVQDLKYATLATVSRCGMVWFSEDVLSTDMIFNNFLARLRSIPLD
EGEDEAQRRRKGKEDEGEEAASPMLQIQRDAATIMQPYFTSNGLVTKALEHAFQLEHIMDLTRLRCLGSLFSMLHQA
CRNVAQYNANHPDFPMQIEQLERYIQRYLVYAILWSLSGDSRLKMRAELGEYIRRITTVPLPTAPNIPIIDYEVSIS
GEWSPWQAKVPQIEVETHKVAAPDVVVPTLDTVRHEALLYTWLAEHKPLVLCGPPGSGKTMTLFSALRALPDMEVVG
LNFSSATTPELLLKTFDHYCEYRRTPNGVVLAPVQLGKWLVLFCDEINLPDMDKYGTQRVISFIRQMVEHGGFYRTS
DQTWVKLERIQFVGACNPPTDPGRKPLSHRFLRHVPVVYVDYPGPASLTQIYGTFNRAMLRLIPSLRTYAEPLTAAM
VEFYTMSQERFTQDTQPHYIYSPREMTRWVRGIFEALRPLETLPVEGLIRIWAHEALRLFQDRLVEDEERRWTDENI
DTVALKHFPNIDREKANSRPILYSNWLSKDYIPVDQEELRDYVKARLKVFYEEELDVPLVLFNEVLDHVLRIDRIFR
QPQGHLLLIGVSGAGKTTLSRFVAWMNGLSVYQIKVHRKYTGEDFDEDLRTVLRRSGCKMEKIAFIMDESNVLDSGF
LERMNTLLANGEVPGLFEGDEYATLMTQCKEGAQKEGLMLDSHEELYKWFTSQVIRNLHVVFTMNPSSEGLKDRAAT
SPALFNRCVLNWFGDWSTEALYQVGKEFTSKMDLEKPNYIVPDYMPVVYDKLPQPPSHREAIVNSCVFVHQTLHQAN
ARLAKRGGRTMAITPRHYLDFINHYANLFHEKRSELEEQQMHLNVGLRKIKETVDQVEELRRDLRIKSQELEVKNAA
ANDKLKKMVKDQQEAEKKKVMSQEIQEQLHKQQEVIADKQMSVKEDLDKVEPAVIEAQNAVKSIKKQHLVEVRSMAN
PPAAVKLALESICLLLGESTTDWKQIRSIIMRENFIPTIVNFSAEEISDAIREKMKKNYMSNPSYNYEIVNRASLAC
GPMVKWAIAQLNYADMLKRVEPLRNELQKLEDDAKDNQQKANEVEQMIRDLEASIARYKEEYAVLISEAQAIKADLA
AVEAKVNRSTALLKSLSAERERWEKTSETFKNQMSTIAGDCLLSAAFIAYAGYFDQQMRQNLFTTWSHHLQQANIQF
RTDIARTEYLSNADERLRWQASSLPADDLCTENAIMLKRFNRYPLIIDPSGQATEFIMNEYKDRKITRTSFLDDAFR
KNLESALRFGNPLLVQDVESYDPVLNPVLNREVRRTGGRVLITLGDQDIDLSPSFVIFLSTRDPTVEFPPDLCSRVT
FVNFTVTRSSLQSQCLNEVLKAERPDVDEKRSDLLKLQGEFQLRLRQLEKSLLQALNEVKGRILDDDTIITTLENLK
REAAEVTRKVEETDIVMQEVETVSQQYLPLSTACSSIYFTMESLKQIHFLYQYSLQFFLDIYHNVLYENPNLKGVTD
HTQRLSIITKDLFQVAFNRVARGMLHQDHITFAMLLARIKLKGTVGEPTYDAEFQHFLRGNEIVLSAGSTPRIQGLT
VEQAEAVVRLSCLPAFKDLIAKVQADEQFGIWLDSSSPEQTVPYLWSEETPATPIGQAIHRLLLIQAFRPDRLLAMA
HMFVSTNLGESFMSIMEQPLDLTHIVGTEVKPNTPVLMCSVPGYDASGHVEDLAAEQNTQITSIAIGSAEGFNQADK
AINTAVKSGRWVMLKNVHLAPGWLMQLEKKLHSLQPHACFRLFLTMEINPKVPVNLLRAGRIFVFEPPPGVKANMLR
TFSSIPVSRICKSPNERARLYFLLAWFHAIIQERLRYAPLGWSKKYEFGESDLRSACDTVDTWLDDTAKASGRQNIS
PDKIPWSALKTLMAQSIYGGRVDNEFDQRLLNTFLERLFTTRSFDSEFKLACKVDGHKDIQMPDGIFFEEFVQWVEL
LPDTQTPSWLGLPNNAERVLLTTQGVDMISKMLKMQMLEDEDDLAYAETEKKTRTDSTSDGRPAWMRTLHTTASNWL
HLIPQTLSHLKRTVENIKDPLFRFFEREVKMGAKLLQDVRQDLADVVQVCEGKKKQTNYLRTLINELVKGILPRSWS
HYTVPAGMTVIQWVSDFSERIKQLQNISLAAASGGAKELKNIHVCLGGLFVPEAYITATRQYVAQANSWSLEELCLE
VNVTTSQGATLDACSFGVTGLKLQGATCNNNKLSLSNAISTALPLTQLRWVKQTNTEKKASVVTLPVYLNFTRADLI
FTVDFEIATKEDPRSFYERGVAVLCTE

SEQ ID NO. 41

MANGVIPPPGGASPLPQVRVPLEEPPLSPDVEEEDDDLGKTLAVSRFGDLISKPPAWDPEKPSRSYSERDFEFHRHT
SHHTHHPLSARLPPPHKLRRLPPTSARHTRRKRKKEKTSAPPSEGTPPIQEEGGAGVDEEEEEEEEEEGESEAEPVE
PPHSGTPQKAKFSIGSDEDDSPGLPGRAAVTKPLPSVGPHTDKSPQHSSSSPSPRARASRLAGEKSRPWSPSASYDL
RERLCPGSALGNPGGPEQQVPTDEAEAQMLGSADLDDMKSHRLEDNPGVRRHLVKKPSRTQGGRGSPSGLAPILRRK
KKKKKLDRRPHEVFVELNELMLDRSQEPHWRETARWIKFEEDVEEETERWGKPHVASLSFRSLLELRRTIAHGAALL
DLEQTTLPGIAHLVVETMIVSDQIRPEDRASVLRTLLLKHSHPNDDKDSGFFPRNPSSSSMNSVLGNHHPTPSHGPD
GAVPTMADDLGEPAPLWPHDPDAKEKPLHMPGGDGHRGKSLKLLEKIPEDAEATVVLVGCVPFLEQPAAAFVRLNEA
VLLESVLEVPVPVRFLFVMLGPSHTSTDYHELGRSIATLMSDKLFHEAAYQADDRQDLLSAISEFLDGSIVIPPSEV
EGRDLLRSVAAFQRELLRKRREREQTKVEMTTRGGYTAPGKELSLELGGSEATPEDDPLLRTGSVFGGLVRDVRRRY
PHYPSDLRDALHSQCVAAVLFIYFAALSPAITFGGLLGEKTEGLMGVSELIVSTAVLGVLFSLLGAQPLLVVGFSGP
LLVFEEAFFKFCRAQDLELYTGRVWVGLWLVVFVLALVAAEGSFLVRYISPFTQEIFAFLISLIFIYETFYKLYKVF
TEHPLLPFYPPEGALEGSLDAGLEPNGSALPPTEGPPSPRNQPNTALLSLILMLGTFFIAFFLRKFRNSRFLGGKAR
RIIGDFGIPISILVMVLVDYSITDTYTQKLTVPTGLSVTSPDKRSWFIPPLGSARPFPPWMMVAAAVPALLVLILIF
METQITALIVSQKARRLLKGSGFHLDLLLIGSLGGLCGLFGLPWLTAATVRSVTHVNALTVMRTAIAPGDKPQIQEV
REQRVTGVLIASLVGLSIVMGAVLRRIPLAVLFGIFLYMGVTSLSGIQLSQRLLLILMPAKHHPEQPYVTKVKTWRM
HLFTCIQLGCIALLWVVKSTAASLAFPFLLLLTVPLRHCLLPRLFQDRELQALDSEDAEPNFDEDGQDEYNELHMPV

SEQ ID NO. 42

DWNVTWNTSNPDFTKCFQNTVLVWVPCFYLWACFPFYFLYLSRHDRGYIQMTPLNKTKTALGFLLWIVCWADLFYSF
WERSRGIFLAPVFLVSPTLLGITMLLATFLIQLERRKGVQSSGIMLTFWLVALVCALAILRSKIMTALKEDAQVDLF
RDITFYVYFSLLLIQLVLSCFSDRSPLFSETIHDPNPCPESSASFLSRITFWWITGLIVRGYRQPLEGSDLWSLNKE
DTSEQVVPVLVKNWKKECAKTRKQPVKVVYSSKDPAQPKESSKVDANEEVEALIVKSPQKEWNPSLFKVLYKTFGPY
FLMSFFFKAIHDLMMFSGPQILKLLIKFVNDTKAPDWQGYFYTVLLFVTACLQTLVLHQYFHICFVSGMRIKTAVIG
AVYRKALVITNSARKSSTVGEIVNLMSVDAQRFMDLATYINMIWSAPLQVILALYLLWLNLGPSVLAGVAVMVLMVP
VNAVMAMKTKTYQVAHMKSKDNRIKLMNEILNGIKVLKLYAWELAFKDKVLAIRQEELKVLKKSAYLSAVGTFTWVC
TPFLVALCTFAVYVTIDENNILDAQTAFVSLALFNILRFPLNILPMVISSIVQASVSLKRLRIFLSHEELEPDSIER
RPVKDGGGTNSITVRNATFTWARSDPPTLNGITFSIPEGALVAVVGQVGCGKSSLLSALLAEMDKVEGHVAIKGSVA
YVPQQAWIQNDSLRENILFGCQLEEPYYRSVIQACALLPDLEILPSGDRTEIGEKGVNLSGGQKQRVSLAPAVYSNA
DIYLFDDPLSAVDAHVGKHIFENVIGPKGMLKNKTRILVTHSMSYLPQVDVIIVMSGGKISEMGSYQELLARDGAFA
EFLRTYASTEQEQDAEENGVTGVSGPGKEAKQMENGMLVTDSAGKQLQRQLSSSSSYSGDISRHHNSTAELQKAEAK
KEETWKLMEADKAQTGQVKLSVYWDYMKAIGLFISFLSIFLFMCNHVSALASNYWLSLWTDDPIVNGTQEHTKVRLS
VYGALGISQGIAVFGYSMAVSIGGILASRCLHVDLLHSILRSPMSFFERTPSGNLVNRFSKELDTVDSMIPEVIKMF
MGSLFNVIGACIVILLATPIAAIIIPPLGLIYFFVQRFYVASSRQLKRLESVSRSPVYSHFNETLLGVSVIRAFEEQ
ERFIHQSDLKVDENQKAYYPSIVANRWLAVRLECVGNCIVLFAALFAVISRHSLSAGLVGLSVSYSLQVTTYLNWLV
RMSSEMETNIVAVERLKEYSETEKEAPWQIQETAPPSSWPQVGRVEFRNYCLRYREDLDFVLRHINVTINGGEKVGI
VGRTGAGKSSLTLGLFRINESAEGEIIIDGINIAKIGLHDLRFKITIIPQDPVLFSGSLRNNLDPFSQYSDEEVWTS
LELAHLKDFVSALPDKLDHECAEGGENLSVGQRQLVCLARALLRKTKILVLDEATAAVDLETDDLIQSTIRTQFEDC
TVLTIAHRLNTIMDYTRVIVLDKGEIQEYGAPSDLLQQRGLFYSMAKDAGLV

SEQ ID NO. 43

FCRAQDLEYLTGRVWVGLWLVVFVLALVAAEGSFLVRYISPFTQEIFAFLISLIFIYETFYKLYKVFTEHPLLPFYP
PEPGGVPGCWSGAKWQLPPTEGPPSPRNQPNTALLSLILMLGTFFIAFFLRKFRNSRFLGGKARRIIGDFGIPISIL
VMVLVDYSITDTYTQKLTVPTGLSVTSPDKRSWFIPPLGSARPFPPWMMVAAAVPALLVLILIFMETQITALIVSQK
ARRLLKGSGFHLDLLLIGSLGGLCGLFGLPWLTAATVRSVTHVNALTVMRTAIAPGDKPQIQEVREQRVTGVLIASL
VGLSIVMGAVLRRIPLAVLFGIFLYMGVTSLSGIQLSQRLLLILMPAKH

SEQ ID NO. 44

MAFWTQLMLLLWKNFMYRRRQPVQLLVELLWPLFLFFILVAVRHSHPPLEHHECHFPNKPLPSAGTVPWLQGLICNV
NNTCFPQLTPGEEPGRLSNFNDSLVSRLLADARTVLGGASAHRTLAGLGKLIATLRAARSTAQPQPTKQSPLEPPML
DVAELLTSLLRTESLGLALGQAQEPLHSLLEAAEDLAQELLALRSLVELRALLQRPRGTSGPLELLSEALCSVRGPS
STVGPSLNWYEASDLMELVGQEPESALPDSSLSPACSELIGALDSHPLSRLLWRRLKPLILGKLLFAPDTPFTRKLM
AQVNRTFEELTLLRNVREVWEMLGPRIFTFMNDSSNVANLQRLLQMQDEGRRQPRPGGRDHMEALRSFLDPGSGGYS
WQDAHADVGHLVGTLGRVTECLSLDKLEAAPSEAALVSRALQLLAEHRFWAGVVFLGPEDSSDPTEHPTPDLGPGHV
RIKIRMDIDVVTRTNKIRDRFWDPGPAADPLTDLRYVWGGFVYLQDLVERAAVRVLSGANPRAGLYLQQMPYPCYVD
DVFLRVLSRSLPLFLTLAWIYSVTLTVKAVVREKETRLRDTMRAMGLSRAVLWLGWFLSCLGPFLLSAALLVLVLKL
GDILPYSIIPGVVFLFLAAFAVATVTQSFLLSAFFSRNLAAACGGLAYFSLYLPYVLCVAWRDRLPAGGRVAASLLS
PVAFGFGCESLALLEEQGEGAQWHNVGTRPTADVFSLAQVSGLLLLDAALYGLATWYLEAVCPGQYGIPEPWNFPFR
RSYWCGPRPPKSPAPCPTPLDPKVLVEEAPPGLSPGVSVRSLEKRFPGSPQPALRGLSLDFYQGHITAFLGHNGAGK
TTTLSILSGLFPPSGGSAFILGHDVRSSMAAIRPHLGVCPQYNVLFDMLTVDEHVWFYGRLKGLSAAVVGPEQDRLL
QDVGLVSKQSVQTRHLSGGMQRKLSVAIAFVGGSQVVILDEPTAGVDPASRRGIWELLLKYREGRTLILSTHHLDEA
ELLGDRVAVVAGGRLCCCGSPLFLRRHLGSGYYLTLVKARLPLTTNEKADTDMEGSVDTRQEKKNGSQGSRVGTPQL
LALVQHWVPGARLVEELPHELVLVLPYTGAHDGSFATLFRELDTRLAELRLTGYGISDTSLEEIFLKVVEECAADTD
MEDGSCGQHLCTGIAGLDVTLRLKMPPQETALENGEPAGSAPETDQGSGPDAVGRVQGWALTRQQLQALLLKRFLLA
RRSRRGLFAQIVLPALFVGLALVFSLIVPPFGHYPALRLSPTMYGAQVSFFSEDAPGDPGRARLLEALLQEAGLEEP
PVQHSSERFSAPEVPAEVAKVLASGNWTPESPSPACQCSRPGARRLLPDCPAAAGGPPPPQAVTGSGEVVQNLTGRN
LSDFLVKTYPRLVRQGLKTKKWVNEVRYGGFSLGGRDPGLPSGQELGRSVEELWALLSPLPGGALDRVLKNLTAWAH
SLDAQDSLKIWFNNKGWHSMVAFVNRASNAILRAHLPPGPARHAHSITTLNHPLNLTKEQLSEGALMASSVDVLVSI
CVVFAMSFVPASFTLVLIEERVTRAKHLQLMGGLSPTLYWLGNFLWDMCNYLVPACIVVLIFLAFQQRAYVAPANLP
ALLLLLLLYGWSITPLMYPASFFFSVPSTAYVVLTCINLFIGINGSMATFVLELFSDQQKLQEVSRILKQVFLIFPH
FCLGRGLIDMVRNQAMADAFERLGDRQFQSPLRWEVVGKNLLAMVIQGPLFLLFTLLLQHRSQLLPQPRVRSLPLLG
EEDEDVARERERVVQGATQGDVLVLRNLTKVYRGQRMPAVDRLCLGIPPGECFGLLGVNGAGKTSTFRMVTGDTLAS
RGEAVLAGHSVAREPSAAHLSMGYCPQSDAIFELLTGREHLELLARLRGVPEAQVAQTAGSGLARLGLSWYADRPAG
TYSGGNKRKLATALALVGDPAVVFLDEPTTGMDPSARRFLWNSLLAVVREGRSVMLTSHSMEECEALCSRLAIMVNG
RFRCLGSPQHLKGRFAAGHTLTLRVPAARSQPAAAFVAAEFPGAELREAHGGRLRFQLPPGGRCALARVFGELAVHG
AEHGVEDFSVSQTMLEEVFLYFSKDQGKDEDTEEQKEAGVGVDPAPGLQHPKRVSQFLDDPSTAETVL

SEQ ID NO. 45

MRLKNLTFIIILIISGELYAEEKPCGFPHVENGRIAQYYYTFKSFYFPMSIDKKLSFFCLAGYTTESGRQEEQTTCT
TEGWSPEPRCFKKCTKPDLSNGYISDVKLLYKIQENNHYGCASGYKTTGGKDEEVVQCLSDGWSSQPTCRKEHETCL
APELYNGNYSTTQKTFKVKDKVQYECATGYYTAGGKKTEEVECLTYGWSLTPKCTKLKCSSLRLIENGYFHPVKQTY
EEGDVVQFFCHENYYLSGSDLIQCYNFGWYPESPVCEGRRNRCPPPPLPINSKIQTHSTTYRHGEIVHIECELNFEI
HGSAEIRCEDGKSTEPPKCIEGQEKVACEEPPFIENGAANLHSKIYYNGDKVTYACKSGYLLHGSNEITCNRGKWTL
PPECVENNENCKHPPVVMNGAVADGILASYATGSSVEYRCNEYYLLRGSKISRCEQGKWSSPPVCLEPCTVNVDYMN
RNNIEMKWKYEGKVLHGDLIDFVCKQGYDLSPLTPLSELSVQCNRGEVKYPLCTRKESKGMCTSPPLIKHGVIISST
VDTYENGSSVEYRCFDHHFLEGSREAYCLDGMWTTPPLCLEPCTLSFTEMEKNNLLLKWDFDNRPHILHGEYIEFIC
RGDTYPAELYITGSILRMQCDRGQLKYPRCIPRQ

SEQ ID NO. 46

MGAAAGRSPHLGPAPARRPQRSLLLLQLLLLVAAPGSTQAQAAPFPELCSYTWEAVDTKNNVLYKINICGSVDIVQC
GPSSAVCMHDLKTRTYHSVGDSVLRSATRSLLEFNTTVSCDQQGTNHRVQSSIAFLCGKTLGTPEFVTATECVHYFE
WRTTAACKKDIFKANKEVPCYVFDEELRKHDLNPLIKLSGAYLVDDSDPDTSLFINVCRDIDTLRDPGSQLRACPPG
TAACLVRGHQAFDVGQPRDGLKLVRKDRLVLSYVREEAGKLDFCDGHSPAVTITFVCPSERREGTIPKLTAKSNCRY
EIEWITEYACHRDYLESKTCSLSGEQQDVSIDLTPLAQSGGSSYISDGKEYLFYLNVCGETEIQFCNKKQAAVCQVK
KSDTSQVKAAGRYHNQTLRYSDGDLTLIYFGGDECSSGFQRMSVINFECNKTAGNDGKGTPVFTGEVDCTYFFTWDT
EYACVKEKEDLLCGATDGKKRYDLSALVRHAEPEQNWEAVDGSQTETEKKHFFINICHRVLQEGKARGCPEDAAVCA
VDKNGSKNLGKFISSPMKEKGNIQLSYSDGDDCGHGKKIKTNITLVCKPGDLESAPVLRTSGEGGCFYEFEWHTAAA
CVLSKTEGENCTVFDSQAGFSFDLSPLTKKNGAYKVETKKYDFYINVCGPVSVSPCQPDSGACQVAKRQVASHDEKT
WNLGLSNAKLSYYDGMIQLNYRGGTPYNNERHTPRATLITFLCDRDAGVGFPEYQEEDNSTYNFRWYTSYACPEEPL
ECVVTDPSTLEQYDLSSLAKSEGGLGGNWYAMDNSGEHVTWRKYYINVCRPLNPVPGCNRYASACQMKYEKDQGSFT
EVVSISNLGMAKTGPVVEDSGSLLLEYVNGSACTTSDGRQTTYTTRIHLVCSRGRLNSHPIFSLNWECVVSFLWNTE
AACPIQTTTDTDQACSIRDPNSGFVFNLNPLNSSQGYNVSGIGKIFMFNVCGTMPVCGTILGKPASGCEAETQTEEL
KNWKPARPVGIEKSLQLSTEGFITLTYKGPLSAKGTADAFIVRFVCNDDVYSGPLKFLHQDIDSGQGIRNTYFEFET
ALACVPSPVDCQVTDLAGNEYDLTGLSTVRKPWTAVDTSVDGRKRTFYLSVCNPLPYIPGCQGDAVGSCLVSEGHSW
NLGVVQMSPQAAANGSLSIMYVNGDKCGNQRFSTRITFECAQISGSPAFQLQDGCEYVFIWRTVEACPVVRVEGDNC
EVKDPRHGNLYDLKPLGLNDTIVSAGEYTYYFRVCGKLSSDVCPTSDKSKVVSSCQEKREPQGFHKVAGLLTQKLTY
ENGLLKMNFTGGDTCHKVYQRSTAIFFYCDRGTQRPVFLKETSDCSYLFEWRTQYACPPFDLTECSFKDGAGNSFDL
SSLSRYSDNWEAITGTGDPEHYLINVCKSLAPQAGTEPCPPEAAACLLGGSKPVNLGRVRDGPQWRDGIIVLKYVDG
DLCPDGIRKKSTTIRFTCSESQVNSRPMFISAVEDCEYTFAWPTATACPMKSNEHDDCQVTNPSTGHLFDLSSLSGR
AGFTAAYSEKGLVYMSICGENENCPPGVGACFGQTRISVGKANKRLRYVDQVLQLVYKDGSPCPSKSGLSYKSVISF
VCRPEARPTNRPMLISLDKQTCTLFFSWHTPLACEQATECSVRNGSSIVDLSPLIHRTGGYEAYDESEDDASDTNPD
FYINICQPLNPMHGVPCPAGAAVCKVPIDGPPIDIGRVAGPPILNPIANEIYLNFESSTPCLADKHFNYTSLIAFHC
KRGVSMGTPKLLRTSECDFVFEWETPVVCPDEVRMDGCTLTDEQLLYSFNLSSLSTSTFKVTRDSRTYSVGVCTFAV
GPEQGGCKDGGVCLLSGTKGASFGRLQSMKLDYRHQDEAVVLSYVNGDRCPPETDDGVPCVFPFIFNGKSYEECIIE
SRAKLWCSTTADYDRDHEWGFCRHSNSYRTSSIIFKCDEDEDIGRPQVFSEVRGCDVTFEWKTKVVCPPKKLECKFV
QKHKTYDLRLLSSLTGSWSLVHNGVSYYINLCQKIYKGPLGCSERASICRRTTTGDVQVLGLVHTQKLGVIGDKVVV
TYSKGYPCGGNKTASSVIELTCTKTVGRPAFKRFDIDSCTYYFSWDSRAACAVKPQEVQMVNGTITNPINGKSFSLG
DIYFKLFRASGDMRTNGDNYLYEIQLSSITSSRNPACSGANICQVKPNDQHFSRKVGTSDKTKYYLQDGDLDVVFAS
SSKCGKDKTKSVSSTIFFHCDPLVEDGIPEFSHETADCQYLFSWYTSAVCPLGVGFDSENPGDDGQMHKGLSERSQA
VGAVLSLLLVALTCCLLALLLYKKERRETVISKLTTCCRRSSNVSYKYSKVNKEEETDENETEWLMEEIQLPPPRQG
KEGQENGHITTKSVKALSSLHGDDQDSEDEVLTIPEVKVHSGRGAGEESSHPVRNAQSNALQEREDDRVGLVRGEKA
RKGKSSSAQQKTVSSTKLVSFHDDSDEDLLHI

SEQ ID NO. 47

LGFSLPPHLLFRPRLDLQFLQRFLQILKVLFPSWSSQNALMFLTLLCLTLLGDFDQFTCNLLYVSWRKDLTEHLHRL
YFRGRAYYTLNVLRDDIDNPDQRISQDVERFCRQLSSMASKLIISPFTLVYYTYQCFQSTGWLGPVSIFGYFILGTV
VNKTLMGPIVMKLVHQEKLEGDFRFKHMQIRVNAEPAAFYRAGHVEHMRTDRRLQRLLQTQRELMSKELWLYIGINT
FDYLGSILSYVVIAIPIFSGVYGDLSPAELSTLVSKNAFVCIYLISCFTQLIDLSTTLSDVAGYTHRIGQLRETLLD
MSLKSQDCEILGESEWGLDTPPGWPAAEPADTAFLLERVSISAPSSDKPLIKDLSLKISEGQSLLITGNTGTGKTSL
LRVLGGLWTSTRGSVQMLTDFGPHGVLFLPQKPFFTDGTLREQVIYPLKEVYPDSGSADDERILRFLELAGLSNLVA
RTEGLDQQVDWNWYDVLSPGEMQRLSFARLFYLQPKYAVLDEATSALTEEVESELYRIGQQLGMTFISVGHRQSLEK
FHSLVLKLCGGGRWELMRIKVE

SEQ ID NO. 48

MAATLILEPAGRCCWDEPVRIAVRGLAPEQPVTLRASLRDEKGALFQAHARYRADTLGELDLERAPALGGSFAGLEP
MGLLWALEPEKPLVRLVKRDVRTPLAVELEVLDGHDPDPGRLLCRVRHERYFLPPGVRREPVRAGRVRGTLFLPPEP
GPFPGIVDMFGTGGGLLEYRASLLAGKGFAVMALAYYNYEDLPKTMETLHLEYFEEAVNYLLSHPEVKGPGVGLLGI
SKGGELCLSMASFLKGITAAVVINGSVANVGGTLRYKGETLPPVGVNRNRIKVTKDGYADIVDVLNSPLEGPDQKSF
IPVERAESTFLFLVGQDDHNWKSEFYANEACKRLQAHGRRKPQIICYPETGHYIEPPYFPLCRASLHALVGSPIIWG
GEPRAHAMAQVDAWKQLQTFFHKHLGGHEGTIPSKV

SEQ ID NO. 49

MPKAPKQQPPEPEWIGDGESTSPSGEAGRQGRNEQRGKREETARFFEELAVEDKQAGEEEKVLKEKEQQQQQQQQQQ
QKKKRDTRKGRRKKDVDDDGEEKELMERLKKLSVPTSDEEDEVPAPKPRGGKKTKGGNVFAALIQDQSEEEEEEEKH
PPKPAKPEKNRINKAVSEEQQPALKGKKGKEEKSKGKAKVRXXXFFLPSQMEYERQVASLKAANAAENDFSVSQAEM
SSRQAMLENASDIKLEKFSISAHGKELFVNADLYIVAGRRYGLVGPNGKGKTTLLKHIANPALSIPPNIDVLLCEQE
VVADETPAVQAVLRADTKRLKLLEEERRLQGQLEQGDDTAAERLEKVYEELRATGAAAAEAKARRILAGLGFDPEMQ
NRPTQKFSGGWRMRVSLARALFMEPTLLMLDEPTNHLDLNAVIWLNNYLQGWRKTLLIVSHDQGFLDDVCTDIIHLD
AQRLHYYRGNYMTFKKMYQQKQKELLKQYEKQEKKLKELKAGGKSTKQAEKQTKEALTRKQQKCRRKNQDEESQEAP
ELLKRPKEYTVRFTFPDPPPLSPPVLGLHGVTFGYQGQKPLFKNLDFGIDMDSRICIVGPNGVGKSTLLLLLTGKLT
PTHGEMRKNHRLKIGFFNQQYAEQLRMEETPTEYLQRGFNLPYQDARKCLGRFGLESHAHTIQICKLSGGQKARVVF
AELACREPDVLILDEPTNNLDIESIDALGEAINEYKGAVIVVSHDARLITETNCQLWVVEEQSVSQIDGDFEDYKRE
VLEALGEVMVSRPRE

SEQ ID NO. 50

KMLSSFLSPQNGTWADTFSLLLALAVALYLGYYWACVLQRPRLVAGPQFLAFLEPHCSITTETFYPTLWCFEGRLQS
IFQVLLQSQPLVLYQSDILQTPDGGQLLLDWAKQPDSSQDPDPTTQPIVLLLPGITGSSQDTYVLHLVNQALRDGYQ
AVVFNNRGCRGEELRTHRAFCASNTEDLETVVNHIKHRYPQAPLLAVGISFGGILVLNHLAQARQAAGLVAALTLSA
CWDSFETTRSLETPLNSLLFNQPLTAGLCQLVERNRKVIEKVVDIDFVLQARTIRQFDERYTSVAFGYQDCVTYYKA
ASPRTKIDAIRIPVLYLSAADDPFSPVCALPIQAAQHSPYVALLITARGGHIGFLEGLLPWQHWYMSRLLHQYAKAI
FQDPEGLPDLRALLPSEDRN

SEQ ID NO. 52

LFTVTVPKELYIIEHGSNVTLECNFDTGSHVNLGAITASLQKVENDTSPHRERATLLEEQLPLGKASFHIPQVQVRD
EGQYQCIIIYGVAWDYKYLTLKVKASYRKINTHILKVPETDEVELTCQATGYPLAEVSWPNVSVPANTSHSRTPEGL
YQVTSVLRLKPPPGRNFSCVFWNTHVRELTLASIDLQSQMEPRTHPTWLLHIFIPFCIIAFIFIATVIALRKQLCQK
LYSSKDVSIHCAKVTLLVPIPTQTTVLQDYSSYGSPTHALSLVPKQDPYGLMR

SEQ ID NO. 53

MARGYGATVSLVLLGLGLALAVIVLAVVLSRHQAPCGPQAFAHAAVAADSKVCSDIGRAILQQQGSPVDATIAALVC
TSVVNPQSMGLGGGVIFTIYNVTTGAQWIGVPGELRGYAEAHRRHGRLPWAQLFQPTIALLRGGHVVAPVLSRFLHN
SILRPSLQASTLRQLFFNGTEPLRPQDPLPWPALATTLETVATEGVEVFYTGRLGQMLVEDIAKEGSQLTLQDLAKF
QPEVVDALEVPLGDYTLYSPPPPAGGAILSFILNVLRGFNFSTESMARPEGRVNVYHHLVETLKFAKGQRWRLGDPR
SHPKLQNASRDLLGETLAQLIRQQIDGRGDHQLSHYSLAEAWGHGTSTSHVSVLGEDGSAVAATSTINTPFGAMVYS
PRTGIILNNELLDLCERCPRGSGTTPSPVETGWVELPEGAGPQFQASVPHPPWCPPS

SEQ ID NO. 54

MAVTLDKDAYYRRVKRLYSNWRKGEDEYANVDAIVVSVGVDEEIVYAKSTALQTWLFGYELTDTIMVFCDDKIIFMA
SKKKVEFLKQIANTKGNENANGAPAITLLIREKNESNKSSFDKMIEAIKESKNGKKIGVFSKDKFPGEFMKSWNDCL
NKEGFDKIDISAVVAYTIAVKEDGELNLMKKAASITSEVFNKFFKERVMEIVDADEKVRHSKLAESVEKAIEEKKYL
AGADPSTVEMCYPPIIQSGGNYNLKFSVVSDKNHMHFGAITCAMGIRFKSYCSNLVRTLMVDPSQEVQENYNFLLQL
QEELLKELRHGVKICDVYNAVMDVVKKQKPELLNKITKNLGFGMGIEFREGSLVINSKNQYKLKKGMVFSINLGFSD
LTNKEGKKPEEKTYALFIGDTVLVDEDGPATVLTSVKKKVKNVGIFLKNEDEEEEEEEITEAEDLLGRGSRAALLTE
RTRNEMTAEEKRRAHQKELAAQLNEEAKRRLTEQKGEQQIQKARKSNVSYKNPSLMPKEPHIREMKIYIDKKYETVI
MPVFGIATPFHIATIKNISMSVEGDYTYLRINFYCPGSALGRNEGNIFPNPEATFVKEITYRASNIKAPGEQTVPAL
NLQNAFRIIKEVQKRYKTREAEEKEKEGIVKQDSLVINLNRSNPKLKDLYIRPNIAQKRMQGSLEAHVNGFRFTSVR
GDKVDILYNNIKHALFQPCDGEMIIVLHFHLKNAIMFGKKRHTDVQFYTEVGEITTDLGKHQHMHDRDDLYAEQMER
EMRHKLKTAFKNFIEKVEALTKEELEFEVPFRDLGFNGAPYRSTCLLQPTSSALVNATEWPPFVVTLDEVELIHFER
VQFHLKNFDMVIVYKDYSKKVTMINAIPVASLDPIKEWLNSCDLKYTEGVQSLNWTKIMKTIVDDPEGFFEQGGWSF
LEPEGEGSDAEEGDSESEIEDETFNPSEDDYEEEEEDSDEDYSSEAEESDYSKESLGSEEESGKDWDELEEEARKAD
RESRYEEEEEQSRSMSRKRKASVHSSGRGSNRGSRHSSAPPKKKRK

SEQ ID NO. 55

SPILCGAATALNCSLCPQDSNLSVHTENPDLTPCFQNSLLAWVPCIYLWVALPCYLLYLRHHCRGYIILSHLSKLKM
VLGVLLWCVSWADLFYSFHGLVHGPAPAPVFFVTPLVVGVTMLLATLLIQYERLQGVQSSGVLIIFWFLCVVCAIVP
FRSKILLAKAEGEISDPFRFTTFYIHFALVLSALILACFREKPPFFSAKNVDPNPYPETSAGFLSRLFFWWFTKMAI
YGYRHPLEEKDLWSLKEEDRSQMVVQQLLEAWRKQEKQTARHKASAAPGKNASGEDEVLLGARPRPRKPSFLKALLA
TFGSSFLISACFKLIQDLLSFINPQLLSILIRFISNPMAPSWWGFLVAGLMFLCSMMQSLILQHYYHYIFVTGVKFR
TGIMGVIYRKALVITNSVKRASTVGEIVNLMSVDAQRFMDLAPFLNLLWSAPLQIILAIYFLWQNLGPSVLAGVAFM
VLLIPLNGAVAVKMRAFQVKQMKLKDSRIKLMSEILNGIKVLKLYAWEPSFLKQVEGIRQGELQLLRTAAYLHTTTT
FTWMCSPFLVTLITLWVYVYVDPNNVLDAEKAFVSVSLFNILRLPLNNLPQLISNLTQASVSLKRIQQFLSQEELDP
QSVERKTISPGYAITIHSGTFTWAQDLPPTLHSLDIQVPKGALVAVVGPVGCGKSSLVSALLGEMEKLEGKVHMKGS
VAYVPQQAWIQNCTLQENVLFGKALNPKRYQQTLEACALLADLEMLPGGDQTEIGEKGINLSGGQRQRVSLARAVYS
DADIFLLDDPLSAVDSHVAKHIFDHVIGPEGVLAGKTRVLVTHGISFLPQTDFIIVLADGQVSEMGPYPALLQRNGS
FANFLCNYAPDEDQGHLEDSWTALEGAEDKEALLIEDTLSNHTDLTDNDPVTYVVQKQFMRQLSALSSDGEGQGRPV
PRRHLGPSEKVQVTEAKADGALTQEEKAAIGTVELSVFWDYAKAVGLCTTLAICLLYVGQSAAAIGANVWLSAWTND
AMADSRQNNTSLRLGVYAALGILQGFLVMLAAMAMAAGGIQAARVLHQALLHNKIRSPQSFFDTTPSGRILNCFSKD
IYVVDEVLAPVILMLLNSFFNAISTLVVIMASTPLFTVVILPLAVLYTLVQRFYAATSRQLKRLESVSRSPIYSHFS
ETVTGASVIRAYNRSRDFEIISDTKVDANQRSCYPYIISNRWLSIGVEFVGNCVVLFAALFAVIGRSSLNPGLVGLS
VSYSLQVTFALNWMIRMMSDLESNIVAVERVKEYSKTETEAPWVVEGSRPPEGWPPRGEVEFRNYSVRYRPGLDLVL
RDLSLHVHGGEKVGIVGRTGAGKSSMTLCLFRILEAAKGEIRIDGLNVADIGLHDLRSQLTIIPQDPILFSGTLRMN
LDPFGSYSEEDIWWALELSHLHTFVSSQPAGLDFQCSEGGENLSVGQRQLVCLARALLRKSRILVLDEATAAIDLET
DNLIQATIRTQFDTCTVLTIAHRLNTIMDYTRVLVLDKGVVAEFDSPANLIAARGIFYGMARDAGLA

SEQ ID NO. 56

PYCSLPRAPLHGFILGQTSTQPGGSIHFGCNAGYRLVGRSMAICTRHPQGYHLWSEAIPLCQALSCGLPEAPKNGMV
FGKEYTVGTKAMYSCSEGYHLQAGAEATAECLDTGLWSNRNVPPQCVPVTCPDVSSISVEHGRWRLIFETQYQFQAQ
LMLICDPGYYYTGQRVIRCQANGKWSLGDSTPTCRILAKQKQPCPSSWGWLTEHLVIILVISCGELPIPPNGHRIGT
LSVYGATAIFSCNSGYTLVGSRVRECMANGLWSGSEVRCLATQTKLHSIFYKLLFDVLSSPSLTKAGHCGTPEPIVN
GHINGENYSYRGSVVYQCNAGFRLIGMSVRICQQDHHWSGKTPFCVPITCGHPGNPVNGLTQGNQFNLNDVVKFVCN
PGYMAEGAARSQCLASGQWSDMLPTCRIINCTDPGHQENSVRQVHASGPHRFSFGTTVSYRCNHGFYLLGTPVLSCQ
GDGTWDRPRPQCLLVSCGHPGSPPHSQMSGDSYTVGAVVRYSCIGKRTLVGNSTRNCGLDGHWTGSLPHCS

SEQ ID NO. 57

PLAFCGSENHSAAYRVDQGVLNNGCFVDALNVVPNVFLLFITFPILFIGWGSQSSKVHIHHSTWLHFPGHNLRWILT
FMLLFVLVCEIAEGILSDGVTESHHLHLYMPAGMAFMAAVTSVVYYHNIETSNFPKLLIALLVYWTLAFITKTIKFV
KFLDHAIGFSQLRFCLTGLLVILYGMLLLVEVNVIRVRRYIFFKTPREVKPPEDLQDLGVRFLQPFVNLLSKGTYWW
MNAFIKTAHKKPIDLRAIGKLPIAMRALTNYQRLCEAFDAQVRKDIQGTQGAPAIWQALSHAFGRRLVLSSTFRILA
DLLGFAGPLCIFGIVDHLGKENDVFQPKTQFLGVYFVSSQEFLANAYVLAVLLFLALLLQRTFLQASYYVAIETGIN
LRGAIQTKIYNKIMHLSTSNLSMGEMTAGQICNLVAIDTNQLMWFFFLCPNLWAMPVQIIVGVILLYYILGVSALIG
AAVIILLAPVQYFVATKLSQAQRSTLEYSNERLKQTNEMLRGIKLLKLYAWENIFRTRVETTRRKEMTSLRAFAIYT
SISIFMNTAIPIAAVLITFVGHVSFFKEADFSPSVAFASLSLFHILVTPLFLLSSVVRSTVKALVSVQKLSEFLSSA
EIREEQCAPHEPTPQGPASKYQAVPLRVVNRKRPAREDCRGLTGPLQSLVPSADGDADNCCVQIMGGYFTWTPDGIP
TLSNITIRIPRGQLTMIVGQVGCGKSSLLLAALGEMQKVSGAVFWSSMPFLPCCSPERETATDLDIRKRGPVAYASQ
KPWLLNATVEENIIFESPFNKQRYKMVIEACSLQPDIDILPHGDQTQIGERGINLSGGQRQRISVARALYQHANVVF
LDDPFSALDIHLSDHLMQAGILELLRDDKRTVVLVTHKLQYLPHADWIIANKDGTIQREGTLKDFQRSECQLFEHWK
TLNNRQDQELEKETVTERKATEPPQGLSRANSSRDGLLQDEEEEEEEAAESEEDDNLSSMLHQRAEIPWRACAKYLS
SAGILLLSLLVFSQLLKHMVLVAIDYWLAKWTDSALTLTPAARNCSLSQECTLDQTVYAMVFTVLCSLGIVLCLVTS
VTVEWTGLKVAKRLHRSLLNRIILAPMRFFETTPLGSILNRFSSDCNTIDQHIPSTLECLSRSTLLCVSALAVISYV
TPVFLVALLPLAIVCYFIQKYFRVASRDLQQLDDTTQLPLLSHFAETVEGLTTIRAFRYEARFQQKLLEYTDSNNIA
SLFLTAANRWLEVRMATPLPPQEYIGACVVLIAAVTSISNSLHRELSAGLVGLGLTYALMVSNYLNWMVRNLADMEL
QLGAVKRIHGLLKTEAESYEGLLAPSLIPKNWPDQGKIQIQNLSVRYDSSLKPVLKHVNALIAPGQKIGICGRTGSG
KSSFSLAFFRMVDTFEGHIIIDGIDIAKLPLHTLRSRLSIILQDPVLFSGTIRFNLDPERKCSDSTLWEALEIAQLK
LVVKALPGGLDAIITEGGENFSQGQRQLFCLAPAFVRKTSIFIMDEATASIDMATENILQKVVMTAFADRTVVTIAH
RVHTILSADLVIVLKRGAILEFDKPEKLLSRKDSVFASFVRADK

SEQ ID NO. 58

MPRNLLYSLLSSHLSPHFSTSVTSAKVAVNGVQLHYQQTGEGDHAVLLLPGMLGSGETDFGPQLKNLNKKLFTVVAW
DPRGYGHSRPPDRDFPADFFERDAKDAVDLMKALKFKKVSLLGWSDGGITALIAAAKYPSYIHKMVIWGANAYVTDE
DSMIYEGIRDVSKWSERTRKPLEALYGYDYFARTCEKWVDGIRQFKHLPDGNICRHLLPRVQCPALIVHGEKDPLVP
RFHADFIHKHVKGSRLHLMPEGKHNLELRFADEFNKLAEDFLQ

SEQ ID NO. 59

MMREWVLLMSVLLCGLAGPTHLFQPSLVLDMAKVLLDNYCFPENLLGMQEAIQQAIKSHEILSISDPQTLASVLTAG
VQSSLNDPRLVISYEPSTPEPPPQVPALTSLSEEELLAWLQRGLRHEVLEGNVGYLRVDSVPGQEVLSMMGEFLVAH
VWGNLMGTSALVLDLRHCTGGQVSGIPYIISYLHPGNTILHVDTIYNRPSNTTTEIWTLPQVLGERYGADKDVVVLT
SSQTRGVAEDIAHILKQMRRAIVVGERTGGGALDLRKLRIGESDFFFTVPVSRSLGPLGGGSQTWEGSGVLPCVGTP
AEQALEKALAILTLRSALPGVVHCLQEVLKDYYTLVDRVPTLLQHLASMDFSTVVSEEDLVTKLNAGLQAASEDPRL
LVRAIGPTETPSWPAPDAAAEDSPGVAPELPEDEAIRQALVDSVFQVSVLPGNVGYLRFDSFADASVLGVLAPYVLR
QVWEPLQDTEHLIMDLRHNPGGPSSAVPLLLSYFQGPEAGPVHLFTTYDRRTNITQEHFSHMELPGPRYSTQRGVYL
LTSHRTATAAEEFAFLMQSLGWATLVGEITAGNLLHTRTVPLLDTPEGSLALTVPVLTFIDNHGEAWLGGGVVPDAI
VLAEEALDKAQEVLEFHQSLGALVEGTGHLLEAHYARPEVVGQTSALLRAKLAQGAYRTAVDLESLASQLTADLQEV
SGDHRLLVFHSPGELVVEEAPPPPPAVPSPEELTYLIEALFKTEVLPGQLGYLRFDAMAELETVKAVGPQLVRLVWQ
QLVDTAALVIDLRYNPGSYSTAIPLLCSYFFEAEPRQRLYSVFDRATSKVTEVWTLPQVAGQRYGSHKDLYILMSHT
SGSAAEAFAHTMQDLQRATVIGEPTAGGALSVGIYQVGSSPLYASMPTQMAMSATTGKAWDLAGVEPDITVPMSEAL
SIAQDIVALRAKVPTVLQTAGKLVADNYASAELGAKMATKLSGLQSRYSRVTSEVALAEILGADLQMLSGDPHLKAA
HIPENAKDRIPGIVPMQIPSPEVFEELIKFSFHTNVLEDNIGYLRFDMFGDGELLTQVSRLLVEHIWKKIMHTDAMI
IDMRFNIGGPTSSIPILCSYFFDEGPPVLLDKIYSRPDDSVSELWTHAQVVGERYGSKKSMVILTSSVTAGTAEEFT
YIMKRLGRALVIGEVTSGGCQPPQTYHVDDTNLYLTIPTARSVGASDGSSWEGVGVTPHVVVPAEEALARAKEMLQH
NQLRVKRSPGLQDHL

SEQ ID NO. 60

MAEVNIIYVTVFILKGITNRPELQAPCFGVFLVIYLVTVLGNLGLITLIKIDTRLHTPMYYFLSHLAFVDLCYSSAI
TPKMMVNFVVERNTIPPHACATQLGCFLTFMITECFLLASMAYDCYVAICSPLHYSTLMSRRVCIQLVAVPYIYSFL
VALFHTVITFRLTYCGPNLINHFYCDDLPFLALSCSDTHMKEILIFAFAGFDMISSSSIVLTSYIFIIAAILRIRST
QGQHKAISTCGSHMVTVTIFYGTLIFMYLQPKSNHSLDTDKMASVFYTVVIPMLNPLIYSLRNKEVKDA

SEQ ID NO. 61

MIQLTATPVSALVDEPVHIRATGLIPFQMVSFQASLEDENGDMFYSQAHYRANEFGEVDLNHASSLGGDYMGVHPMG
LFWSLKPEKLLTRLLKRDVMNRPFQVQVKLYDLELIVNNKVASAPKASLTLERWYVAPGVTRIKVREGRLRGALFLP
PGEGLFPGVIDLFGGLGGLLEFRASLLASRGFASLALAYHNYEDLPRKPEVTDLEYFEEAANFLLRHPKVFGSGVGV
VSVCQGVQIGLSMAIYLKQVTATVLINGTNFPFGIPQVYHGQIHQPLPHSAQLISTNALGLLELYRTFETTQVGASQ
YLFPIEEAQGQFLFIVGEGDKTINSKAHAEQAIGQLKRHGKNNWTLLSYPGAGHLIEPPYSPLCCASTTHDLRLHWG
GEVIPHAAAQEHAWKEIQRFLRKHLIPDVTSQL

SEQ ID NO. 62

ISPQSRDAKPNPEEPIDEDEDIQTERIRTATALTTSILDEVELKGCSSVLGHLGYCPQENVLWPMLTLREHLEVYAA
VKGLRKADARLAIARLVSAFKLHEQLNVPVQKLTAGITRKLCFVLSLLGNSPVLLLDEPSTGIDPTGQQQMWQAIQA
VVKNTERGVLLTTHNLAEAEALCDRVAIMVSGRLRCIGSIQHLKNKLGKDYILELKVKETSQVTLVHTEILKLFPQA
AGQERYSSLLTYKLPVADVYPLSQTFHKLEA

SEQ ID NO. 63

WNTSNPDFTKCFQNTVLVWVPCFYLWACFPFYFLYLSRHDRGYIQMTPLNKTKTALGFLLWIVCWADLFYSFWERSR
GIFLAPVFLVSPTLLGITMLLATFLIQLERRKGVQSSGIMLTFWLVALVCALAILRSKIMTALKEVDLFRDITFYVY
FSLLLIQLVLSCFSDRSPLFSETIHDPNPCPESSASFLSRITFWWITGLIVRGYRQPLEGSDLWSLNKEDTSEQVVP
VLVKNWKKECAKTRNSSGSGESCSANTEALFPAPTCHKSFQALSLLLCRLLIKFVNDTKAPDWQGYFYTVLLFVTAC
LQTLVLHQYFHICFVSGMRIKTAVIGAVYRKALVITNSARKSSTVGEIVNLMSVDAQRFMDLATYINNIWSAPLQVI
LALYLLWLVVAPDVLTAVSSKVAHMKSKDNRIKLMNEILNGIKVLKLYAWELAFKDKVLAIRQEELKVLKKSAYLSA
VGTFTWVCTPFLVALCTFAVYVTIDENNILDAQTAFVSLALFNILRFPLNILPMVISSIVQVQGEAGATSERGPWGS
RPRKHGTRQASFSVAEPGVLCRFSITFSIPEGALVAVVGQVGCGKSSLLSALLAEMDKVEGHVAIKGSVAYVPQQAW
IQNDSLRENILFGCQLEEPYYRSVIQACALLPDLEILPSGDRTEIGEKGVNLSGGQKQRVSLARAVYSNADIYLFDD
PLSAVDAHVGKHIFENVIGPKGMLKNKSCLISCDLQVKLSVYWDYMKAIGLFISFLSIFLFMCNHVSALASNYWLSL
WTDDPIVNGTQEHTKVRLSVYGALGISQGIAVFGYSMAVSIGGILASRCLHVDLLHSILRSPMSFFERTPSGNLVNR
FSKELDTVDSMIPEVIKMFMGSLFNVIGACIVILLATPIAAIIIPPLGLIYFFVQRFYVASSRQLKRLESVSRSPVY
SHFNETLLGVSVIRAFEEQERFIHQSDLKVDENQKAYYPSIVANRWLAVRLECVGNCIVLFAALFAVISRHSLSAGL
VGLSVSYSLQVTTYLNWLVRNSSEMETNIVAVERLKEYSETEKEAPWQIQETAPPSSWPQVGRVEFRNYCLRYREDL
DFVLRHINTINGGEKVGIVGRTGAGKSSLTLGLFRINESAEGEIIIDGINIAKIIGLHDLRFKITIIPQDPVLFSGS
LRMNLDPFSQYSDEEVWTSLELAHLKDFVSALPDKLDHECAEGGENLSVGQRQLVCLARALLRKTKILVLDEATAAV
DLETDDLIQSTIRTQFEDCTVLTIAHRLNTIMDYTRVIVLDKGEIQEYGAPSDLLQQRGLFYSMAKDAGLV

SEQ ID NO. 64

HRLIGHSSAECILSGNTAHWSTKPPICQRIPCGLPPTIANGDFISTNRENFHYGSVVTYRCNLGSRGRKVFELVGEP
SIYCTSNDDQVGIWSGPAPQCIIPNKCTPPNVENGILVSDNRSLFSLNEVVEFRCQPGFVMKGPRRVKCQALNKWEP
ELPSCSRVCQPPPEILHGEHTPSHQDNFSPGQEVFYSCEPGYDLRGAASLHCTPQGDWSPEAPRCAVKSCDDFLGQL
PHGRVLFPLNLQLGAKVSFVCDEGFRLKGSSVSHCVLVGMRSLWNNSVPVCEHIFCPNPPAILNGRHTGTPSGDIPY
GKEISYTCDPHPDRGMTFNLIGESTIRCTSDPHGNGVWSSPAPRCELSVRAGHCKTPEQFPFASPTIPINDFEFPVG
TSLNYECRPGYFGKMFSISCLENLVWSSVEDNCRRKSCGPPPEPFNGMVHINTDTQFGSTVNYSCNEGFRLIGSPST
TCLVSGNNVTWDKKAPICEIISCEPPPTISNGDFYSNNRTSFHNGTVVTYQCHTGPDGEQLFELVGERSIYCTSKDD
QVGVWSSPPPRCISTNKCTAPEVENAIRVPGNRSFFTLTEIIRFRCQPGFVMVGSHTVQCQTNGRWGPKLPHCSRVC
QPPHILHGEHTLSHQDNFSPGQEVFYSCEPSYDLRGAASLHCTPQGDWSPEAPRCTVKSCDDFLGQLPHGRVLLPLN
LQLGAKVSFVCDEGFRLKGRSASHCVLAGMKALWNSSVPVCER

SEQ ID NO. 65

MRGPPAWPLRLLEPPSPAEPGRLLPVACVWAAASRVPGSLSPFTGLRPARLWGAGPALLWGVGAARRWRSGCRGGGP
GASRGVLGLARLLGLWARGPGSCRCGAFAGPGAPRLPRARFPGGPAAAAWAGDEAWRRGPAAPPGDKGRLRPAAAGL
PEARKLLGLAYPERRRLAAAVGFLTMSSVISMSAPFFLGKIIDVIYTNPTVDYSDNLTRLCLGLSAVFLCGAAANAI
RVYLMQTSGQRIVNRLRTSLFSSILRQEVAFFDKTRTGELINRLSSDTALLGRSVTENLSDGLRAGAQASVGISMMF
FVSPNLATFVLSVVPPVSIIAVIYGRYLRKLTKVTQDSLAQATQLAEERIGNVRTVRAFGKEMTEIEKYASKVDHVM
QLARKEAFARAGFFGATGLSGNLIVLSVLYKGGLLMGSAHMTVGELSSFLMYAFWVGISIGGLSSFYSELMKGLGAG
GRLWELLEREPKLPFNEGVILNEKSFQGALEFKNVHFAYPARPEVPIFQDFSLSIPSGSVTALVGPSGSGKSTVLSL
LLRLYDPASGTISLDGHDIRQLNPVWLRSKIGTVSQEPILFSCSIAENIAYGADDPSSVTAEEIQRVAEVANAVAFI
RNFPQGFNTVVGEKGVLLSGGQKQRIAIARALLKNPKILLLDEATSALDAENEYLVQEALDRLMDGRTVLVIAHRLS
TIKNANMVAVLDQGKITEYGKHEELLSKPNGIYRKLMNKQSFISA

SEQ ID NO. 66

GILTWYIEAVHPGMYGLPRPWYFPLQKSYWLGSGRTEAWEWSWPWARTPRLSVMEEDQACAMESRRFEETRGMEEEP
THLPLVVCVDKLTKVYKDDKKLALNKLSLNLYENQVVSFLGHNGAGKTTHHNVLFDRLTVEEHLWFYSRLKSMAQEE
IRREMDKMIEDLELSNKRHSLVQTLSGGMKRKLSVAIAFVGGSRAIILDEPTAGVDPYARRAIWDLILKYKPGRTIL
LSTHHMDEADLLGDRIAIISHGKLKCCGSPLFLKGTYGDGYRLTLVKRPAEPGGPQEPGLASSPPGRAPLSSCSELQ
MSQFIRKHVASCLLVSDTSTELSYILPSEAAKKGAFERLFQHLERSLDALHLSSFGLMDTTLEEVFLKVSEEDQSLE
NSEADVKESRKDVLPGAEGPASGEGHAGNLARCSELTQSQASLQSASSVGSARGDEGAGYTDVYGDYRPLFDNPQDP
DNVSLQEVEAEALSRVGQGSRKLDGGWLKVRQFHGLLVKRFHCARRNSKALFSQILLPAFFVCVAMTVALSVPEIGD
LPPLVLSPSQYHNYTQPRGNFIPYANEERREYRRHPAGASLVGGASEGAGTALVGQAGEGAGLARGGDWQPHLTLIW
GRGGEDLGPESAAPAPPCAGITVTNHPMNKTSASLSLDYLLQGTDVVIAIFIIVAMSFVPASFVVFLVAEKSTKAKH
LQFVSGCNPIIYWLANYVWDMLNYLVPATCCVIILFVFDLPAYTSPTNFPAVLSLFLLYGWSITPIMYPASFWFEVP
SSAYVFLIVINLFIGITATVATFLLQLFEHDKDLKVVNSYLKSCFLIFPNYNLGHGLMEMAYNEYINEYYAKIGQFD
KMKSPFEWDIVTRGLVAMAVEGVVGFLLTIMCQYNFLRRPQRMPVSTKPVEDDVDVASERQRVLRGDSDNDMCFGLL
GVNGAGKTSTFKMLTGDESTTGGEAFVNGHSVLKELLQVQQSLGYCPQCDALFDELTAREHLQLYTRLRGISWKDEA
RVVKWALEKLELTKYADKPAGTYSGGNKRKLSTAIALIGYPAFIFLDEPTTGMDPKARRFLWNLILDLIKTGRSVVL
TSHSMEECEALCTRLAIMVNGRLRCLGSIQHLKNRFGDGYMITVRTKSSQSVKDVTRFFNRNFPEAMLKERHHTKVQ
YQLKSEHISLAQVFSKMEQVSGVLGIEDYSVSQTTLDNVFVNFAKKQSDNLEQQETEPPSALQSPLGCLLSLLRPRS
APTELRALVADEPEDLDTEDEGLISFEEERAQLSFNTDTLC

SEQ ID NO. 67

MGPGRPAPAPWPRHLLRCVLLLGCLHLGRPGAPGDAALPEPNIFLIFSHGLQGCLEAQGGQVRVTPACNTSLPAQRW
KWVSRIRLFNLGTMQCLGTGWPGTNTTASLGMYECDREALNLRWHCRTLGDQLSLLLGARTSNISKPGTLERGDQTR
SGQWRIYGSEEDLCALPYHEVYTIQGNSHGKPCTIPFKYDNQWFHGCTSTGREDGHLWCATTQDYGKDERWGFCPIK
SNDCETFWDKDQLTDSCYQFNFQSTLSWREAWASCEQQGADLLSITEIHEQTYINGLLTGYSSTLWIGLNDLDTSGG
WQWSDNSPLKYLNWESDQPDNPSEENCGVIRTESSGGWQNRDCSIALPYVCKKKPNATAEPTPPDRWANVKVECEPS
WQPFQGHCYRLQAEKRSWQESKRACLRGGGDLVSIHSMAELEFITKQIKQEVEELWIGLNDLKLQMNFEWSDGSLVS
FTHWHPFEPNNFRDSLEDCVTIWGPEGRWNDSPCNQSLPSICKKAGQLSQGAAEEDHGCRKGWTWHSPSCYWLGEDQ
VTYSEARRLCTDHGSQLVTITNRFEQAFVSSLIYNWEGEYFWTALQDLNSTGSFFWLSGDEVMYTHWNRDQPGYSRG
GCVALATGSAMGLWEVKNCTSFRARYICRQSLGTPVTPELPGPDPTPSLTGSCPQGWASDTKLRYCYKVFSSERLQD
KKSWVQAQGACQELGAQLLSLASYEEEHFVANMLNKIFGESEPEIHEQHWFWIGLNRRDPRGGQSWRWSDGVGFSYH
NFDRSRHDDDDIRGCAVLDLASLQWVANQCDTQLDWICKIPRGTDVREPDDSPQGRREWLRFQEAEYKFFEHHSTWA
QAQRICTWFQAELTSVHSQAELDFLSHNLQKFSRAQEQHWWIGLHTSESDGRFRWTDGSIINFISWAPGKPRPVGKD
KKCVYMTASREDWGDQRCLTALPYICKRSNVTKETQPPDLPTTALGGCPSDWIQFLNKCFQVQGQEPQSRVKWSEAQ
FSCEQQEAQLVTITNPLEQAFITASLPNVTFDLWIGLHASQRDFQWVEQEPLMYANWAPGEPSGPSPAPSGNKPTSC
AVVLHSPSAHFTGRWDDRSCTEETHGFICQKGTDPSLSPSPAALPPAPGTELSYLNGTFRLLQKPLRWHDALLLCES
HNASLAYVPDPYTQAFLTQAARGLRTPLWIGLAGEEGSRRYSWVSEEPLNYVGWQDGEPQQPGGCTYVDVDGAWRTT
SCDTKLQGAYCGVSSGPPPPRRISYHGSCPQGLADSAWIPFREHCYSFHMELLLGHKEARQRCQRAGGAVLSILDEM
ENVFVWEHLQSYEGQSRGAWLGMNFNPKGGTLVWQDNTAVNYSNWGPPGLGPSMLSHNSCYWIQSNSGLWRPGACTN
ITMGVVCKLPRAEQSSFSPSALPENPAALVVVLMAVLLLLALLTAALILYRRRQSIERGAFEGARYSRSSSSPTEAT
EKNILVSDMEMNEQQE

SEQ ID NO. 68

MKYILVTGGVISGIGKGIIASSIGTILKSCGLRVTAIKIDPYINIDAGTFSPYEHGEVFVLNDGGEVDLDLGNYERF
LDINLYKDNNITTGKIYQHVINKERRGDYLGKTVQVVPHITDAVQEWVMNQAKVPVDGNKEEPQICLGGTIGDIEGM
PFVEAFRQFQFKAKRENFCNIHVSLVPQLSATGEQKTKPTQNSVRALRGLGLSPDLIVCRSSTPIEMAVKEKISMFC
HVNPEQVICIHDVSSTYRVPVLLEEQSIVKYFKERLHLPIGDSASNLLFKWRNMADRYERLQKICSIALVGKYTKLR
DCYASVFKALEHSALAINHKLNLMYIDSIDLEKITETEDPVKFHEAWQKLCKADGILVPGGFGIRGTLGKLQAISWA
RTKKIPFLGVCLGMQLAVIEFARNCLNLKDADSTEFRPNAPVPLVIDMPEHNPGNLGGTMRLGIRRTVFKTENSILR
KLYGDVPFIEERHRHRFEVNPNLIKQFEQNDLSFVGQDVDGDRMEIIELANHPYFVGVQFHPEFSSRPMKPSPPYLG
LLLAATGNLNAYLQQGCKLSSSDRYSDASDDSFSEPRIAELEIS

SEQ ID NO. 69

SPILCGAATALNCSLCPQDSNLSVHTENPDLTPCFQNSLLAWVPCIYLWVALPCYLLYLRHHCRGYIILSHLSKLKM
VLGVLLWCVSWADLFYSFHGLVHGRAPAPVFFVTPLVVGVTMLLATLLIQYERLQGVQSSGVLIIFWFLCVVCAIVP
FRSKILLAKAEGEISDPFRFTTFYIHFALVLSALILACFREKPPFFSAKNVDPNPYPETSAGFLSRLFFWWFTKMAI
YGYRHPLEEKDLWSLKEEDRSQMVVQQLLEAWRKQEKQTARHKASAAPGKNASGEDEVLLGARPRPRKPSFLKALLA
TFGSSFLISACFKLIQDLLSFINPQLLSILIRFISNPMAPSWWGFLVAGLMFLCSMMQSLILQHYYHYIFVTGVKFR
TGIMGVIYRKALVITNSVKRASTVGEIVNLMSVDAQRFMDLAPFLNLLWSAPLQIILAIYFLWQNLGPSVLAGVAFM
VLLIPLNGAVAVKMRAFQVKQMKLKDSRIKLMSEILNGIKVLKLYAWEPSFLKQVEGIRQGELQLLRTAAYLHTTTT
FTWMCSPFLVTLITLWVYYYVDPNNVLDAEKAFVSVSLFNILRLPLNMLPQLISNLTQASVSLKRIQQFLSQEELDP
QSVERKTISPGYAITIHSGTFTWAQDLPPTLHSLDIQVPKGALVAVVGPVGCGKSSLVSALLGEMEKLEGKVHMKGS
VAYVPQQAWIQNCTLQENVLFGKALNPKRYQQTLEACALLADLEMLPGGDQTEIGEKGINLSGGQRQRVSLARAVYS
DADIFLLDDPLSAVDSHVAKHIFDHVIGPEGVLAGKTRVLVTHGISFLPQTDFIIVLADGQVSEMGPYPALLQRNGS
FANFLCNYAPDEDQGHLEDSWTALEGAEDKEALLIEDTLSNHTDLTDNDPVTYVVQKQFMRQLSALSSDGEGQGRPV
PRRHLGPSEKVQVTEAKADGALTQEEKAAIGTVELSVFWDYAKAVGLCTTLAICLLYVGQSAAAIGANVWLSAWTND
AMADSRQNNTSLRLGVYAALGILQGFLVMLAAMAMAAGGIQAARVLNQALLHNKIRSPQSFFDTTPSGRILNCFSKD
IYVVDEVLAPVILMLLNSFFNAISTLVVIMASTPLFTVVILPLAVLYTLVQRFYAATSRQLKRLESVSRSPIYSHFS
ETVTGASVIRAYNRSRDFEIISDTKVDANQRSCYPYIISNRSEAASLAPCSSRNSQQALWCSGSLSLLSPKQKTGPA
LPLPHFLLI

SEQ ID NO. 70

AFHQGSLILCLALQSDRLLIKGGRIINDDQSLYADVYLEDGLIKQIGENLIVPGGVKTIEANGRNVIPGGIDVNTYL
QKPSQGMTAADDFFQGTRAALVGGTTMIIDHVVPEPGSSLLTSFEKWHEAADTKSCCDYSLHVDITSWYDGVREELE
VLVQDKGVNSFQVYMAYKDVYQMSDSQLYEAFTFLKGLGAVILVHAENGDLIAQEQKRILEMGITGPEGHALSRPEE
LEAEAVFRAITIAGRINCPVYITKVMSKSAADIIALARKKGPLVFGEPIAASLGTDGTHYWSKNWAKAAAFVTSPPL
SPDPTTPDYLTSLLACGDLQVTGSGHCPYSTAQKAVGKDNFTLIPEGVNGIEERMTVVWDKAVATGKMDENQFVAVT
STNAAKIFNLYPRKGRIAVGSDADVVIWDPDKLKTITAKSHKSAVEYNIFEGMECHGSPLVVISQGKIVFEDGNINV
NKGMGRFIPRKAFPEHLYGRVKIRNKVFGLQGVSRGMYDGPVYEVPATPKYATPAPSAKSSPSKHQPPPIRNLHQSN
FSLSGAQIDDNNPRRTGHRIVAPPGGRSNITSLG

SEQ ID NO. 71

MQRALPGARQHLGAILASASVVVKALCAAVLFLYLLSFAVDTGCLAVTPGYLFPPNFWIWTLATHGLMEQHVWDVAI
SLTTVVVAGRLLEPLWGALELLIFFSVVNVSVGLLGAFAYLLTYMASFNLVYLFTVRIHGALGFLGGVLVALKQTMG
DCVVLRVPQVRVSVMPMLLLALLLLLRLATLLQSPALASYGFGLLSSWVYLRFYQRHSRGRGDMADHFAFATFFPEI
LQPVVGLLANLVHSLLVKVKICQKTVKRYDVGAPSSITISLPGTDPQDAERRRQLALKALNERLKRVEDQSIWPSMD
DDEEESGAKVDSPLPSDKAPTPPGKGAAPESSLITFEAAPPTL

SEQ ID NO. 72

MPVLSRPRPWRGNTLKRTAVLLALAAYGAHKVYPLVRQCLAPARGLQAPAGEPTQEASGVAAAKAGMNRVFLQRLLW
LLRLLFPRVLCRETGLLALHSAALVSRTFLSVYVARLDGRLARCIVRKDPRAFGWQLLQWLLIALPATFVNSAIRYL
EGQLALSFRSRLVAHAYRLYFSQQTYYRVSNMDGRLRNPDQSLTEDVVAFAASVAHLYSNLTKPLLDVAVTSYTLLR
AARSRGAGTAWPSAIAGLVVFLTANVLRAFSPKFGELVAEEARRKGELRYMHSRVVANSEEIAFYGGHEVELALLQR
SYQDLASQINLILLERLWYVMLEQFLMKYVWSASGLLMVAVPIITATGYSESDAEAVKKAALEKKEEELVSERTEAF
TIARNLLTAAADAIERIMSSYKEVTELAGYTAPVHEMFQVFEDVQRCHFKRPRELEDAQAGSGTIGRSGVRVEGPLK
IRGQVVDVEQGIICENIPIVTPSGEVVVASLNIRVEEGMHLLITGPNGCGKSSLFRILGGLWPTYGGVLYKPPPQRM
FYIPQRPYMSVGSLRDQVIYPDSVEDMQRKGYSEQDLEAILDVVHLHHILQREGGWEAMCDWKDVLSGGEKQRIGMA
RNFYHRPKYALLDECTSAVSIDVEGKIFQAAKDAGIALLSITHRPSLWKYHTHLLQFDGEGGWKFEKLDSAARLSLT
EEKQRLEQQLAGIPKMQRRLQELCQILGEAVAPAHVPAPSPQGPGGLQGAST

SEQ ID NO. 73

MDLDVVNMFVIAGGTLAIPILAFVASFLLWPSALIRIYYWYWRRTLGMQVRYVHHEDYQFCYSFRGRPGHKPSILML
HGFSAHKDMWLSVVKFLPKNLHLVCVDMPGHEGTTRSSLDDLSIDGQVKRIHQFVECLKLNKKPFHLVGTSMGGQVA
GVYAAYYPSDVSSLCLVCPAGLQYSTDNQFVQRLKELQGSAAVEKIPLIPSTPEEMSEMLQLCSYVRFKVPQQILQG
LVDVRIPHNNFYRKLFLEIVSEKSRYSLHQNMDKIKVPTQIIWGKQDQVLDVSGADMLAKSIANCQVELLENCGHSV
VMERPRKTAKLIIDFLASVHNTDNNK

SEQ ID NO. 74

SDRLLIRGGRIVNDDQSFYADVHVEDGLIKQIGENLIVPGGIKTIDAHGLMVLPGGVDVHTRLQMPVLGMTPADDFC
QGTKAALAGGTTMILDHVFPDTGVSLLAAYEQWRERADSAACCDYSLHVDITRWHESIKEELEALVKEKGVNSFLVF
MAYKDRCQCSDSQMYEIFSIIRDLGALAQVHAENGDIVEEEQKRLLELGITGPEGHVLSHPEEVEAEAVYRAVTIAK
QANCPLYVTKVMSKGAADAIAQAKRRGVVVFGEPITASLGTDGSHYWSKNWAKAAAFVTSPPVNPDPTTADHLTCLL
SSGDLQVTGSAHCTFTTAQKAVGKDNFALIPEGTNGIEERMSMVWEKCVASGKMDENEFVAVTSTNAAKIFNFYPRK
GRVAVGSDADLVIWNPKATKIISAKTHNLNVEYNIFEGVECRGAPAVVISQGRVALEDGKMFVTPGAGRFVPRKTFP
DFVYKRIKARNRLAEIHGVPRGLYDGPVHEVMVPAKPGSGAPARASCPGKISVPPVRNLHQSGFSLSGSQADDHIAR
RTAQKIMAPPGGRSNITSLS

SEQ ID NO. 75

MARRSVLYFILLNALINKGQACFCDHYAWTQWTSCSKTCNSGTQSRRRQIVVDKYYQENFCEQICSKQETRECNWQR
CPINCLLGDFGPWSDCDPCIEKQGTSNFHYLNHLFTSFFHLDSSFIRIHKVMKVLNFTTKAKDLHLSDVFLKALNHL
PLEYNSALYSRIFDDFGTHYFTSGSLGGVYDLLYQFSSEELKNSGLTEEEAKNCVRIETKKRVLFAKKTKVEHRCTT
NKLSEKHEGSFIQGAEKSISLIRGGRSEYGAALAWEKGSSGLEEKTFSEWLESVKENPAVIDFELAPIVDLVRNIPC
AVTKRNNLRKALQEYAAKFDPCQCAPCPNNGRPTLSGTECLCVCQSGTYGENCEKQSPDYKSNAVDGQWGCWSSWST
CDATYKRSRTRECNNPAPQRGGKRCEGEKRQEEDCTFSIMENNGQPCINDDEEMKEVDLPEIEADSGCPQPVPPENG
FIRNEKQLYLVGEDVEISCLTGFETVGYQYFRCLPDGTWRQGDVECQRTECIKPVVQEVLTITPFQRLYRIGESIEL
TCPKGFVVAGPSRYTCQGNSWTPPISNSLTCEKDTLTKLKGHCQLGQKQSGSECICMSPEEDCSHHSEDLCVFDTDS
NDYFTSPACKFLAEKCLNNQQLHFLHIGSCQDGRQLEWGLERTRLSSNSTKKESCGYDTCYDWEKCSASTSKCVCLL
PPQCFKGGNQLYCVKMGSSTSEKTLNICEVGTIRCANRKMEILHPGKCLA

SEQ ID NO. 76

MERKNQTAITEFIILGFSNLNELQFLLFTIFFLTYFCTLGGNILIILTTVTDPHLHTPMYYFLGNLAFIDICYTTSN
VPQMNVHLLSKKKSISYVGCVVQLFAFVFFVGSECLLLAAMAYDRYIAICNPLRYSVILSKVLCNQLAASCWAAGFL
NSVVHTVLTFCLPFCGNNQINYFFCDIPPLLILSCGNTSVNELALLSTGVFIGWTPFLCIVLSYICIISTILRIQSS
EGRRKAFSTCASHLAIVFLFYGSAIFTYVRPISTYSLKKDRLVSVLYSVVTPMLNPIIYTLRNKDIKEAVKTIGSKW
QPPISSLDSKLTY

SEQ ID NO. 77

MDPGKDKEGVPQPSGPPARKKFVIPLDEDEVPPGVAKPLFRSTQSLPTVDTSAQAAPQTYAEYAISQPLEGAGATCP
TGSEPLAGETPNQALKPGAKSNSIIVSPRQRGNPVLKFVRNVPWEFGDVIPDYVLGQSTCALFLSLRYHNLHPDYIH
GRLQSLGKNFALRVLLVQVDVKDPQQALKELAKMCILADCTLILAWSPEEAGRYLETYKAYEQKPADLLMEKLEQDF
VSRVTECLTTVKSVNKTDSQTLLTTFGSLEQLIAASREDLALCPGLPPQKARRLFDVLHEPFLKVP

SEQ ID NO. 78

MLANSASVRILIKGGKVVNDDCTHEADVYIENGIIQQVGRELMIPGGAKVIDATGKLVIPGGIDTSTHFHQTFMNAT
CVDDFYHGTKAALVGGTTMIIGHVLPDKETSLVDAYEKCRGLADPKVCCDYALHVGITWWAPKVKAEMETLVREKGV
NSFQMFMTYKDLNWNMLRDSELYQVLHACKDIGAIARVHAENGELVAEASLQPRILDGGTPGAKEALDLGITGPEGI
EISRPEELEAEATHRVITIANRTHCPIYLVNVSSISAGDVIAAAKMQGKVVLAETTTAHATLTGLHYYHQDWSHAAA
YVTVPPLRLDTNTSTYLMSLLANDTLNIVASDHRPFTTKQKANGKEDFTKIPHGVSGVQDRMSVIWERGVVGGKMDE
NRFVAVTSSNAAKLLNLYPRKGRIIPGADADVVVWDPEATKTISASTQVQGGDFNLYENMRCHGVPLVTISRGRVVY
ENGVFMCAEGTGKFCPLRSFPDTVYKKLVQREKTLKVRGVDRTPYLGDVAVVVHPGKKEMGTPLADTPTRPVTRHGG
MRDLHESSFSLSGSQIDDHVPKRASARILAPPGGRSSGIW

SEQ ID NO. 79

VTAVAQQNQGEVPEPQDMKVAEVLFDAADANAIEEVNLAYENVKEVDGLDVSKEGTEAWEAAMKRYDERIDRVETRI
TARLRDQLGTAKNANEMFRIFSRFNALFVRPHIRGAIREYQTQLIQRVKDDIESLHDKFKVQYPQSQACKMSHVRDL
PPVSGSIIWAKQIDRQLTAYNKRVEDVLGKGWENHVEGQKLKQDGDSFRMKLNTQEIFDDWARKVQQRNLGVSGRIF
TIESTRVRGRTGNVLKLKVNFLPEIITLSKEVRNLKWLGFRVPLAIVNKAHQANQLYPFAISLIESVRTYERTCEKV
EERNTISLLVAGLKKEVQALIAEGIALVWESYKLDPYVQRLAETVFNFQEKVCSHVIL

SEQ ID NO. 80

MSDSVILRSIKKFGEENDGFESDKSWWSLNPYVFLIRLQDEKKGDGVRVGFFQLFRFSSSTDIWLMFVGSLCAFLHG
IAQPGVLLIFGTMTDVFIDYDVELQELQIPGKACVNNTIVWTNSSLNQNMTNGTRCGLLNIESEMIKFASYYAGIAV
AVLITGYIQICFWVIAAARQIQKMRKFYFRRIMRMEIGWFDCNSVGELNTRFSDDINKINDAIADQMALFIQRMTST
ICGFLLGFFRGWKLTLVIISVSPLIGIGAATIGLSVSKFTDYELKAYAKAGVVADEVISSMRTVAAFGGEKREVERY
EKNLVFAQRWGIRKGIVMGFFTGFVWCLIFLCYALAFWYGSTLVLDEGEYTPGTLVQIFLSVIVGALNLGNASPCLE
AFATGRAAATSIFETIDRKPIIDCMSEDGYKLDRIKGEIEFHNVTFHYPSRPEVKILNDLNMVIKPGEMTALVGPSG
AGKSTALQLIQRFYDPCEGMVTVDGHDIRSLNIQWLRDQIGIVEQEPVLFSTTIAENIRYGREDATMEDIVQAAKEA
NAYNFIMDLPQQFDTLVGEGGGQMSGGQKQRVAIARALIRNPKILLLDMATSALDNESEAMVQEVLSKIQHGHTIIS
VAHRLSTVRAADTIIGFEHGTAVERGTHEELLERKGVYFTLVTLQSQGNQALNEEDIKGKCFFPILVLDATEDDMLA
RTFSRGSYQDSLRASIRQRSKSQLSYLVHEPPLAVVDHKSTYEEDRKDKDIPVQEEVEPAPVRRILKFSAPEWPYML
VGSVGAAVNGTVTPLYAFLFSQILGTFSIPDKEEQRSQINGVCLLFVANGCVSLFTQFLQGYAFAKSGELLTKRLRK
FGFRAMLGQDIAWFDDLRNSPGALTTRLATDASQVQGAAGSQIGMIVNSFTNVTVAMIIAFSFSWKLSLVILCFFPF
LALSGATQTRMLTGFASRDKQALEMVGQITNEALSNIRTVAGIGKERRFIEALETELEKPFKTAIQKANIYGFCFAF
AQCIMFIANSASYRYGGYLISNEGLHFSYVFRVISAVVLSATALGRAFSYTPSYAKAKISAARFFQLLDRQPPISVY
NTAGEKWDNFQGKIDFVDCKFTYPSRPDSQVLNGLSVSISPGQTLAFVGSSGCGKSTSIQLLERFYDPDQGKVMIDG
HDSKKVNVQFLRSNIGIVSQEPVLFACSIMDNIKYGDNTKEIPMERVIAAAKQAQLHDFVMSLPEKYETNVGSQGSQ
LSRGEKQRIAIARAIVRDPKILLLDEATSALDTESEKTVQVALDKAREGRTCIVIAHRLSTIQNADIIAVMAQGVVI
EKGTHEELMAQKGAYYKLVTTGSPIS

SEQ ID NO. 81

MDLEAAKNGTAWRPTSAEGDFELGISSKQKRKKTKTVKMIGVLTLFRYSDWQDKLFMSLGTIMAIAHGSGLPLMMIV
FGEMTDKFVDTAGNFSFPVNFSLSLLNPGKILEEEMTRYAYYYSGLGAGVLVAAYIQVSFWTLAAGRQIRKIRQKFF
HAILRQEIGWFDINDTTELNTRLTDDISKISEGIGDKVGMFFQAVATFFAGFIVGFIRGWKLTLVIMAISPILGLSA
AVWAKILSAFSDKELAAYAKAGAVAEEALGAIRTVIAFGGQNKELERYQKHLENAKEIGIKKAISANISMGIAFLLI
YASYALAFWYGSTLVISKEYTIGNANTVFFSILIGAFSVGQAAPCIDAFANARGAAYVIFDIIDNNPKIDSFSERGH
KPDSIKGNLEFNDVHFSYPSRANVKILKGLNLKVQSGQTVALVGSSGCGKSTTVQLIQRLYDPDEGTINIDGQDIRN
FNVNYLREIIGVVSQEPVLFSTTIAENICYGRGNVTMDEIKKAVKEANAYEFIMKLPQKFDTLVGERGAQLSGGQKQ
RIAIARALVRNPKILLLDEATSALDTESEAEVQAALDKAREGRTTIVIAHRLSTVRNADVIAGFEDGVIVEQGSHSE
LMKKEGVYFKLVNMQTSGSQIQSEEFELNDEKAATRMAPNGWKSRLFRHSTQKNLKNSQMCQKSLDVETDGLEANVP
PVSFLKVLKLNKTEWPYFVVGTVCAIANGGLQPAFSVIFSEIIAIFGPGDDAVKQQKCNIFSLIFLFLGIISFFTFF
LQGFTFGKAGEILTRRLRSMAFKANLRQDMSWFDDHKNSTGALSTRLATDAAQVQGATGTRLALIAQNIANLGTGII
ISFIYGWQLTLLLLAVVPIIAVSGIVEMKLLAGNAKRDKKELEAAGKIATEAIENIRTVVSLTQERKFESMYVEKLY
GPYRNSVQKAHIYGITFSISQAFMYFSYAGCFRFGAYLIVNGHMRFRDVILVFSAIVFGAVALGHASSFAPDYAKAK
LSAAHLFMLFERQPLIDSYSEEGLKPDKFEGNITFNEVVFNYPTRANVPVLQGLSLEVKKGQTLALVGSSGCGKSTV
VQLLERFYDPLAGTVLLDGQEAKKLNVQWLRAQLGIVSQEPILFDCSIAENIAYGDNSRVVSQDEIVSAAKAANIHP
FIETLPHKYETRVGDKGTQLSGGQKQRIAIARALIRQPQILLLDEATSALDTESEKVVQEALDKAREGRTCIVIAHR
LSTIQNADLIVVFQNGRVKEHGTHQQLLAQKGIYFSMVSVQAGTQNL

SEQ ID NO. 82

MDLEGDRNGGAKKKNFFKLNNKSEKDKKEKKPTVSVFSMFRYSNWLDKLYMVVGTLAAIIHGAGLPLMMLVFGEMTD
IFANAGNLEDLMSNITNRSDINDTGFFMNLEEDMTRYAYYYSGIGAGVLVAAYIQVSFWCLAAGRQIHKIRKQFFHA
IMRQEIGWFDVHDVGELNTRLTDDVSKINEGIGDKIGMFFQSMATFFTGFIVGFTRGWKLTLVILAISPVLGLSAAV
WAKILSSFTDKELLAYAKAGAVAEEVLAAIRTVIAFGGQKKELERYNKNLEEAKRIGIKKAITANISIGAAFLLIYA
SYALAFWYGTTLVLSGEYSIGQVLTVFFSVLIGAFSVGQASPSIEAFANARGAAYEIFKIIDNKPSIDSYSKSGHKP
DNIKGNLEFRNVHFSYPSRKEVKILKGLNLKVQSGQTVALVGNSGCGKSTTVQLMQRLYDPTEGMVSVDGQDIRTIN
VRFLREIIGVVSQEPVLFATTIAENIRYGRENVTMDEIEKAVKEANAYDFIMKLPHKFDTLVGERGAQLSGGQKQRI
AIARALVRNPKILLLDEATSALDTESEAVVQVALDKARKGRTTIVIAHRLSTVRNADVIAGFDDGVIVEKGNHDELM
KEKGIYFKLVTMQTAGNEVELENAADESKSEIDALEMSSNDSRSSLIRKRSTRRSVRGSQAQDRKLSTKEALDESIP
PVSFWRIMKLNLTEWPYFVVGVFCAIINGGLQPAFAIIFSKIIGVFTRIDDPETKRQNSNLFSLLFLALGIISFITF
FLQGFTFGKAGEILTKRLRYMVFRSMLRQDVSWFDDPKNTTGALTTRLANDAAQVKGAIGSRLAVITQNIANLGTGI
IISFIYGWQLTLLLLAIVPIIAIAGVVEMKMLSGQALKDKKELEGSGKIATEAIENFRTVVSLTQEQKFEHMYAQSL
QVPYRNSLRKAHIFGITFSFTQAMMYFSYAGCFRFGAYLVAHKLMSFEDVLLVFSAVVFGAMAVGQVSSFAPDYAKA
KISAAHIIMIIEKTPLIDSYSTEGLMPNTLEGNVTFGEVVFNYPTRPDIPVLQGLSLEVKKGQTLALVGSSGCGKST
VVQLLERFYDPLAGKVLLDGKEIKRLNVQWLRAHLGIVSQEPILFDCSIAENIAYGDNSRVVSQEEIVRAAKEANIH
AFIESLPNKYSTKVGDKGTQLSGGQKQRIAIARALVRQPHILLLDEATSALDTESEKVVQEALDKAREGRTCIVIAH
RLSTIQNADLIVVFQNGRVKEHGTHQQLLAQKGIYFSMVSVQAGTKRQ

SEQ ID NO. 83

MLLTVYCVRRDLSEVTFSLQVDADFELHNFRALCELESGIPAAESQIVYAERPLTDNHRSLASYGLIWGDVVILRQK
ENADPRPPVQFPNLPRIDFSSIAVPGTSSPRQRQPPGTQQSHSSPGEITSSPQGLDNPALLRDMLLANPHELSLLKE
RNPPLAEALLSGDLEKFSRVLVEQQQDRARREQERIRLFSADPFDLEAQAKIEEDIRQQNIEENMTIAMEEAPESFG
QVVMLYINCKVNGHPVKAFVDSGAQMTIMSQACAERCNIMRLVDRRWAGIAKGVGTQKIIGRVHLAQVQIEGDFLPC
SFSILEEQPMDMLLGLDMLKRHQCSIDLKKNVLVIGTTGSQTTFLPEGELPECARLAYGAGREDVRPEEI

SEQ ID NO. 84

QTGPSVTVTCTEGKWNKQCRIKCEDTAPHAVLPSGSECATSCLDHNSESIILPMNVTVRDIPHWLNPTRVEVSDQGH
L

SEQ ID NO. 85

MSATLILEPPGRCCWNEPVRIAVRGLAPEQRVTLRASLRDEKGALFRAHARYCADARGELDLERAPALGGSFAGLEP
MGLLWALEPEKPFWRFLKRDVQIPFVVELEVLDGHDPEPGRLLCQAQHERHFLPPGVRRQSVRAGRVRATLFLPPGP
GPFPGIIDIFGIGGGLLEYRASLLAGHGFATLALAYYNFEDLPNNMDNISLEYFEEAVCYMLQHPQVKGPGIGLLGI
SLGADICLSMASFLKNVSATVSINGSGISGNTAINYKHSSIPPLGYDLRRIKVAFSGLVDIVDIRNALVGGYKNPSM
IPIEKAQGPILLIVGQDDHNWRSELYAQTVSERLQAHGKEKPQIICYPGTGHYIEPPYFPLCPASLHRLLNKHVIWG
GEPRAHSKAQEDAWKQILAFFCKHLGGTQKTA

SEQ ID NO. 86

ILHGEHTLSHQDNFSPGQEVFYSCEPSYDLRGAASLHCTPQGDWSPEAPRCTVKSCDDFLGQLPHGRVLLPLNLQLG
AKVSFVCDEGFRLKGRSASHCVLAGMKALWNSSVPVCEXXMIKTVFLFFSLPISNNAHENPKEVAIHLHSQGGSSVH
PRTLQTNEENSRYIHTEFKMFSTTQISKMETGLEYDIALANNECKNSYSLVTREIFVIHYIDCALPFPGIICGLPPT
IANGDFTSISREYFHYGSVVTYHCNLGSRGKKVFELVGEPSIYCTSKDDQVGIWSGPAPQCIIPNKCTPPNVENGIL
VSDNRSLFSLNEVVEFRCQPGFGMKGPSHVKCQALNKWEPELPSCSRVCQPPPDVLHAERTQRDKDNFSPGQEVFYS
CEPGYDLRGSTYLHCTPQGDWSPAAPRCEVKSCDDFLGQLPNGHVLFPLNLQLGAKVDFVCDEGFQLKGSSASYCVL
AGMESLWNSSVPVCERVTFQANLSPSSVQYLTHDTLRTEESSDYSTWLQNIFFPTGKSCETPPVPVNGMVHVITDIH
VGSRINYSCTTGHRLIGHSSAECILSGNTAHWSMKPPICQRIPCGLPPNITNGYFISTDREYFHYGSVVTYHCNLGS
RGRKVFELVGEPSIYCTSKDDQVVVWSGPVPQCIIPNKCTPPNVENGILVSDNRSLFSLNEVVEFRCQPGFVMKGPH
RVQCQALNKWEPELPSCSRGYSKRNSPITNKYSGTVLSTMCQPPPEILHGEHTLSHQDNFLPGQEVFYSCEPSYDLR
GAASLHCMPQGDWTPEAPRCTGASLSPSHGSLTPVVLFFLLVKSCDDFLGQLPHGRVLFPLNLQLGAKVSFVCDEGS
ASHCVLAGTKALWNSSVPVCEQIFCPNPPAILNGRHTGTPPGDIPYGKEVSYTCDPHPDRGMTFNLIGESTIRRTSE
PHGNGVWSSPAPRCELPVGADQCNVPEWLPFARPTNLTDDFEFPIGTYLNYECRPGYSGRPFSIICLKNSVWTSAKD
KCKRKSCRNPPDPVNGMAHVIKDIQFRSQIKYSCPKGYRLIGSSSATCIISGNTVIWDNKTPVCD

SEQ ID NO. 87

ICCPDDPQPAKDQLATVPKDIPLDCDCVLTGEDILGEVANRTAQGLEGLVSDSACTVGTIDAEQLSDTDSVQMFLEL
EKECLCEEGVTPLVELQNQISSEGLAASQDAENLLVISHFSGAALEKEQHLGLLHVRAKDYDTRLDCGYFNTLDSSQ
VPNAVELIAHVDIMRDTSTVSKEECEKVPFSPRTAEFKSRQPADLDSLEKLDPGGLLNSDHRVSHEEKLSGFIASEL
AKDNGSLSQGDCSQTEGNGEECIERVTFSFAFNHELTDVTSGPEVEVLYESNLLTDEIHLESGNVTVNQENNSLTSM
GNVVTCELSVEKVCDEDGEAKELDYQATLLEDQAPAHFHRNFPEQVFQDLQRKSPESEILSLHLLVEELRLNPDGVE
TVNDTKPELNVASSEGGEMERRDSDSFLNIFPEKQVTKAGNTEPVLEEWIPVLQRPSRTAAVPTVKDALDAALPSPE
EGTSIAAVPAPEGTAVVAALVPFPHEDILVASIVSLEEEDVTAAAVSAPERATVPAVTVSVPEGTAAVAAVSSPEET
APAVAAAITQEGMSAVAGFSPEWAALAVTVPITEEDAAAVPTPEVAAIPAASVPTPEVPAIPAAAVPPMEEVSPIGV
PFLGVSAHTDSVPISEEGTPVLEEASSTGMWIKEDLDSLVFGIKEVTSTVLHGKVPLAATAGLNSDE

SEQ ID NO. 88

SEGNKRRLSTAIALMGRSSVIFLDEPSTGMDPVARRLLWNMVTKTRESGKAIVMTSHSMEECDALCTSLAIMVQGKF
TCLGSPQHLKSKFGNIYILKVKVKTEDKLEDFKCYVATTFPGEIAMVTVFLLLLLKVFGILEEAKEQFDLEDYSVSQ
ITLEQVFLTFANPEKASSDD

SEQ ID NO. 89

DCGPPPELPFAFPINPLYDTEFKTGTTLKYTCHPGHGKINSSRLICDAKDSWNYSIFCAIAKCEPPPDIRNGKHSGG
DQEFYTYASSVTYSCNPYFSLIGNVSISCTVENETIGVWSPNPPICE

SEQ ID NO. 90

ISKDRKERVHQGMVRAATVGYGILREGGSAVDAVEGAVVALEDDPEFNAGCGSVLNTNGEVEMDASIMDGKDLSAGA
VSAVQCIANPIKLARLVMEKTPHCFLTDQGAAQFAAAMGVPEIPGEKLVTERNKKRLEKEKHEKGAQKTDCQKNLGT
VGAVALDCKGNVAYATSTGGIVNKMVGRVGDSPCLGAGGYADNDIGAVSTTGHGESILKVNLARLTLFHIEQGKTVE
EAADLSLGYMKSRVKGLGGLIVVSKTGDWVAKWTSTSMPWAAAKDGKLHFGIDPDDTTITDLP

SEQ ID NO. 91

LQLGAKVSFVCDEGFRLKGRSASHCVLAGMKALWNSSVPVCERIICGLPPTIANGDFTSISREYFHYGSVVTYHCNL
GSRQKKVFELVGEPSIYCTSKDDQVGIWSGPAPQCIIPNKCTPPNVENGILVSDNRSLFSLNEVVEFRCQPGFGMKG
PSHVKCQALNKWEPELPSCSRVCQPPPDVLHAERTQRDKDNFSPGQEVFYSCEPGYDLRGSTYLHCTPQGDWSPAAP
RCEVKSCDDFLGQLPNGHVLFPLNLQLGAKVDFVCDEGFQLKGSSASYCVLAGMESLWNSSVPVCEQIFCPNPPAIL
NGRHTGTPPGDIPYGKEVSYTCDPHPDRGMTFNLIGESTIRRTSEPHGNGVWSSPAPRCELPVGADQCNVPEWLPFA
RPTNLTDDFEFPIGTYLNYECRPGYSGRPFSIICLKNSVWTSAKDKCKRKSCRNPPDPVNGMAHVIKDIQFRSQIKY
SCPKG

SEQ ID NO. 92

HGRVLLPLNLQLGAKVSFVCDEGFRLKGRSASHCVLAGMKALWNSSVPVCERIICGLPPTIANGDFTSISREYFHYG
SVVTYHCNLGSRGKKVFELVGEPSIYCTSKDDQVGIWSGPAPQCIIPNKCTPPNVENGILVSDNRSLFSLNEVVEFR
CQPGFGMKGPSHVKCQALNKWEPELPSCSRVCQPPPDVLHAERTQRDKDNFSPGQEVFYSCEPGYDLRGSTYLHCTP
QGDWSPAAPRCEVKSCDDFLGQLPNGHVLFPLNLQLGAKVDFVCDEGFQLKGSSASYCVLAGMESLWNSSVPVCEQI
FCPNPPAILNGRHTGTPPGDIPYGKEVSYTCDPHPDRGMTFNLIGESTIRRTSEPHGNGVWSSPAPRCELPVGAGQY
PLPHILNGFRICSEVEVFEYLNAVTDSCDPAPGPDPFSLIGESTIYCGDNSVWNHAAPECK

1. A method of cleaving a peptide bond in a desired protein comprising contacting the desired protein with a protease comprising a sequence selected from the group consisting of SEQ ID NOs. 1-92, under conditions wherein the protease hydrolyzes at least one peptide bond in the desired protein.

2. A method for identifying a compound that modulates the activity of a protease comprising: (a) contacting a protease having an amino acid sequence selected from the group consisting SEQ ID NOs. 1-92, or a functional fragment or variant thereof, with a test compound; (b) measuring the activity of the protease before and after the contacting step; and (c) determining whether the test compound modulates the activity of the protease.

3. The method according to claim 2, wherein step (c) comprises measuring the level of proteolytic activity or hydrolytic activity.

4. The method according to claim 2, wherein step (c) comprises measuring the amount of product generated from cleavage of a substrate by the protease.

5. The method according to claim 2, wherein the test compound is an inhibitor of proteolytic function of the protease.

6. A method for identifying a compound that modulates the activity of a protease in a cell comprising: (a) expressing, in a cell, a protease having an amino acid sequence selected from the group consisting SEQ ID NOs 1-92; (b) exposing the cell to a test compound; and (c) monitoring an alteration in cell phenotype or proteolytic activity.

7. A method for treating a disease or disorder by administering to a patient in need of such treatment a compound that modulates the activity of a protease having an amino acid sequence selected from the group consisting of SEQ ID NOs 1-92.

8. The method according to claim 7, wherein the patient is a mammal.

9. The method according to claim 7, wherein the mammal is selected from the group consisting of a human, primate, rat, mouse, rabbit, pig, cattle, sheep, goat, cat and dog.

10. The method according to claim 9, wherein the mammal is a human.

11. The method according to claim 7, wherein the disease or disorder is selected from the group consisting of cancers, immune-related diseases and disorders, cardiovascular disease, brain or neuronal-associated diseases, and metabolic disorders.

12. The method according to claim 11, wherein said disease or disorders are cancers.

13. The method according to claim 12, wherein the cancers involve at least one gene selected from the group consisting of: GD2, Lewis-Y, 72 kd glycoprotein, C017-1A, TAG-72, CSAg-P, 45kd glycoprotein, HT-29 ag, NG2, A33, 38kd gp, MUC-1, CEA, EGFR, HER2, HER3, HER4, HN-1 ligand, CA125, Syndecan-1, Lewis-X, PgP, FAP, EDG Receptors, ED-B, Laminin-5, Cox-2, AlphaVbeta3 integrin, AlphaVbeta5 integrin, uPAR, Endoglin and the Folate receptor osteopontin.

14. The method according to claim 13, wherein the gene is at least one of CEA, TAG72, EDB, FAP, AlphaVbeta3 integrin and AlphaVbeta5 integrin.

15. The method according to claim 12, wherein said cancers are cancers of tissues or cancers of hematopoietic origin

16. The method according to claim 7, wherein the compound modulates protease activity in vitro.

17. A method for treating a disease or disorder, comprising administering to a patient in need of such treatment a pharmaceutical composition comprising a. protease having an amino acid sequence selected from the group consisting of SEQ ID NOs 1-92.

18. A method for detection of a protease in a sample as a diagnostic tool for a disease or disorder, comprising (a) contacting the sample with a nucleic acid probe which hybridizes under hybridization assay conditions to a nucleic acid target, the target encoding a protease having an amino acid sequence selected from the group consisting of SEQ ID NOs 1-92, or fragments thereof, or the complements of the sequences and fragments thereof; and (b) detecting the presence or amount of the probe:target region hybrid as an indication of the disease.

19. A method for detection of a protease in a sample as a diagnostic tool for a disease or disorder, comprising: (a) comparing a nucleic acid target region encoding a protease in a sample, wherein the protease has an amino acid sequence selected from the group consisting of SEQ ID NOs 1-92 or one or more fragments thereof, with a control nucleic acid target region encoding the protease polypeptide, or one or more fragments thereof; and (b) detecting differences in nucleotide or predicted amino acid sequence or amount between the target region and the control target region, as an indication of said disease or disorder.

20. An antibody that binds to a part of a protein comprising the sequence described in any one of SEQ ID NOs. 1-92.


SEQUENCE LISTING (92 sequences)

SEQ ID NO: 1 - protein, 303 aa, Homo sapiens
NMAILCAMVVGVGLIAGLAVGLTRSCDSSGDGGLGTVPAPSHLPSSTASPSGPPAQDQDICPSSEDESGQWKNFTAELRQPGPDLHVKPLLEEDTYTGTVSISINLSTPTRHLWLHLGESRITWLPDTRHLWLHLQETRITWLPEMKRPSGDQVQIRRCFEYKKQEYVVVEAEEESGDGLYLLTMEFAGWLNSSLLGFTYTENGQVKSIAATDHEPTDARKFFPCFDKPNKKATYTISVTHPKEYEALSHMPVAKEESVDDKWNQTTFKKSVPMSMYLVCFAVHQFHTVKTISDIGKPVSLII

SEQ ID NO: 2 - protein, 407 aa, Homo sapiens
  VARIANT 230: Xaa = Any Amino Acid
MSPPLLLLPLLLLLPLLNVEPAGATLDPVWIPLRQVHPGRRTLNLLRGWGKPAELPKLGPSPGDKPASVPLSKFLDAQYFGEIGLGTPPQNFTVAFDTGSSNLWVPSRRCHFFSVPCWFHHRFNPNASSSFKPSGTKFAIQYGTGRVDGILSEDKLTIGGIKGASVIFGEALWESSLVFTVSRPDGILRLGFPILSVEGVRPPLDVLVEQGLLDKPVFSFYFNRCWGGGXGCAMYCRIVRLEDPLDTGTPVIVGPTEEIGPCMQPLGESLLAGEYIIRCSEIPKLPAVSLLIGGVWFNLTAQDYVIQFAQGDVRLCLSGFRALDIASPPVPVWILGDVFLGAYVTVFDRGDMKSGARVGLARARPGADLGRRETQAQYRGCRPGDHAHRVALALLSKNPIFPLNEPA

SEQ ID NO: 3 - protein, 122 aa, Homo sapiens
MDTAAKAIILEQSGKNQGYRDADIRSFWPEGGVCLPGSPDVLESGVCMKAVCKRVAVEGVDVIFSRDAGRYVCDYTYYLSLHHGKGCAALIHVPPLSRGLPASLLGRALRVIIQEMLEEVGK

SEQ ID NO: 4 - protein, 393 aa, Homo sapiens
IYVSSWAVQVSQGNREVERLARKFGFVNLGPIFPDGQYFHLRHRGVVQQSLTPHWGHRLHLKKNPKVQWFQQQTLQRRVKRSVVVPTDPWFSKQWYMNSEAQPDLSILQAWSQGLSGQGIVVSVLDDGIEKDHPDLWANYDPLASYDFNDYDPDPQPRYTPSKENRHGTRCAGEVAAMANNGFCGVGVAFNARIGGVRMLDGTITDVIEAQSLSLQPQHIHIYSASWGPEDDGRTVDGPGILTREAFRRGVTKGRGGLGTLFIWASGNGGLHYDNCNCDGYTNSIHTLSVGSTTQQGRVPWYSEACASTLTTTYSSGVATDPQIVTTDLHHGCTDQHTGTSASAPLAAGMIALALEANPFLTWRDMQHLVVRASKPAHLQAEDWRTNGVGRQG

SEQ ID NO: 5 - protein, 153 aa, Homo sapiens
LVGYAIQYGCIAHCASEYVGGVVMCSGPSMEPTIQNSDTVFAQNLSRHFDSIQRGDIVIAKSPSDPTSNICKRVTGLEGDKILTTSPSDFFKSYSYVPVGHVWLEGDLQNSTDSSYYGPIPYELIRGRIFFIRPLSDFGFLCASLNGHRFSDD

SEQ ID NO: 6 - protein, 396 aa, Homo sapiens
MLITVYCVRRDLSEVTFSLQVSPDFELRNFKVLCEAESRVPVEEIQIIHMERLLIEDHCSLGSYGLKDGDIVVLLQKDNVGPRAPGRAPNQPRVDFSGIAVPGTSSSRPQHPGQQQQRTPAAQRSQGLASGEKVAGLQGLGSPALIRSMLLSNPHDLSLLKERNPPLAEALLSGSLETFSQVLMEQQREKALREQERLRLYTADPLDREAQAKIEEEIRQQNIEENMNIAIEEAPESFGQVTMLYINCKVNGHPLKAFVDSGAQMTIMSQACAERCNIMRLVDRRWAGVAKGVGTQRIIGRVHLAQIQIEGDFLQCSFSILEDQPMDMLLGLDMLRRHQCSIDLKKNVLVIGTTGTQTYFLPEGELPLCSRMVSGQDESSDKEITHSVMDSGRKEH

SEQ ID NO: 7 - protein, 469 aa, Homo sapiens
HGRVLLPLNLQLGAKVSFVCDEGFRLKGRSASHCVLAGMKALWNSSVPVCERIICGLPPTIANGDFTSISREYFHYGSVVTYHCNLGSRGKKVFELVGEPSIYCTSKDDQVGIWSGPAPQCIIPNKCTPPNVENGILVSDNRSLFSLNEVVEFRCQPGFGMKGPSHVKCQALNKWEPELPSCSRVCQPPPDVLHAERTQRDKDNFSPGQEVFYSCEPGYDLRGSTYLHCTPQGDWSPAAPRCEVKSCDDFLGQLPNGHVLFPLNLQLGAKVDFVCDEGFQLKGSSASYCVLAGMESLWNSSVPVCERKSCETPPVPVNGMVHVITDIHVGSRINYSCTTGSASHCVLAGTKALWNSSVPVCEQIFCPNPPAILNGRHTGTPPGDIPYGKEVSYTCDPHPDRGMTFNLIGESTIRRTSEPHGNGVWSSPAPRCELPVGAVIFSTHLITLFYCLGTLLGTIIFILIIIFLY

SEQ ID NO: 8 - protein, 556 aa, Homo sapiens
GGGSPGWGCAGIPDSAPGAGVLQAGAVGPARGGQGAEEVGESAGGGEERRVRHPQAPALRLLNRKPQGGSGEIKTPENDLQRGRLSRGPRTAPPAPGMGDRSGQQERSVPHSPGAPVGTSAAAVNGLLHNGFHPPPVQPPHVCSRGPVGGSDAAPQRLPLLPELQPQPLLPQHDSPAKKCRLRRRMDSGRKNRPPFPWFGMDIGGTLVKLVYFEPKDITAEEEQEEVENLKSIRKYLTSNTAYGKTGIRDVHLELKNLTMCGRKGNLHFIRFPSCAMHRFIQMGSEKNFSSLHTTLCATGGGAFKFEEDFRMIADLQLHKLDELDCLIQGLLYVDSVGFNGKPECYYFENPTNPELCQKKPYCLDNPYPMLLVNMGSGVSILAVYSKDNYKRVTGTSLGGGTFLGLCCLLTGCETFEEALEMAAKGDSTNVDKLVKDIYGGDYERFGLQGSAVASSFGNMMSKEKRDSISKEDLARATLVTITNNIGSIARMCALNENIDRVVFVGNFLRINMVSMKLLAYAMDFWSKGQLKALFLEHEGYFGAVGALLELFKMTD

SEQ ID NO: 9 - protein, 993 aa, Homo sapiens
MRPVALLLLPSLLALLAHGLSLEAPTVGKGQAPGIEETDGELTAAPTPEQPERGVHFVTTAPTLKLLNHHPLLEEFLHEGLEKGDEELRPALSFQPDPPAPFTPSALPRLANQDSRPVFTSPTPAMGAVPTQPQSKEGPWSPESESPMLRITAPLPPGPSMAVPTLGPGEIASTTPPSRAWTPTQEGPGDMGRPWVAEVVSQGAGIGIQGTITSSTASGDDEETTTTTTIITTTITTVQTPGPCSWNFSGPEGSLDSPTDLSSPTDVGLDCFFYISVYPGYGVEIKVQNISLREGETVTVEGLGGPDPLPLANQSFLLRGQVIRSPTHQAALRFQSLPPPAGPGTFHFHYQAYLLSCHFPRRPAYGDVTVTSLHPGGSARFHCATGYQLKGARHLTCLNATQPFWDSKEPVCIAACGGVIRNATTGRIVSPGFPGNYSNNLTCHWLLEAPEGQRLHLHFEKVSLAEDDDRLIIRNGDNVEAPPVYDSYEVEYLPIEGLLSSGKHFFVELSTDSSGAAAGMALRYEAFQQGHCYEPFVKYGNFSSSTPTYPVGTTVEFSCDPGYTLEQGSIIIECVDPHDPQWNETEPACRAVCSGEITDSAGVVLPTEPEPYGRGQDSIWGVHVEEDKRIMLDIRVLRIGPGDVLTFYDGDDLTARVLGQYSGPRSHFKLFTSMADVTIQFQSDPGTSVLGYQQGFVIHFFEVPRNDTCPELPEIPNGWKSPSQPELVHGTVVTYQCYPGYQVVGSSVLMCQWDLTWSEDLPSCQRVTSCHDPGDVEHSRRLISSPKFPVGATVQYICDQGFVLMGSSILTCHDRQAGSPKWSDRAPKCLLEQLKPCHGLSAPENGARSPEKQLHPAGATIHFSCAPGYVLKGQASIKCVPGHPSHWSDPPPICRAASLDGFYNSRSLDVAKAPAASSTLDAAHIAAAIFLPLVAMVLLVGGVYFYFSRLQGKSSLQLPRPRPRPYNRITIESAFDNPTYETGSLSFAGDERI

SEQ ID NO: 10 - protein, 276 aa, Homo sapiens
EKALALTGNQGIEAAMDWLMEHEDDPDVDEPLETPLVTYPGEPTSSEQGGLEGSGSAAGEGKPALSEEERQEQTKRMLELVAQKQREREEREEREALERERQRRRQGQELSAARQRLQEDEMRRAAEERRREKAEELAARQRVREKIERDKAERAKKYGGSVGSQPPPVAPEPGPVPSSPSQEPPPKREYDQCRIQVRLPDGTSLTQTFRAREQLAAVRLYVELHRGEELGGGQDPVQLLSGFPRRAFSEADMERPLQELGLVPSAVLIVAKKCPS

SEQ ID NO: 11 - protein, 241 aa, Homo sapiens
SLHLSERADWQYSQRELDAVEVFFSRTARDNRLGCMFVRCAPSSRYTLLFSHGNAVDLGQMCSFYIGLGSRINCNIFSYDYSGYGVSSGKPSEKNLYADIDAAWQALRTRYGVSPENIILYGQSIGTVPTVDLASRYECAAVILHSPLMSGLRVAFPDTRKTYCFDAFPSIDKISKVTSPVLVIHGTEDEVIDFSHGLAMYERCPRAVEPLWVEGAGHNDIELYAQYLERLKQFIHELPNS

SEQ ID NO: 12 - protein, 420 aa, Homo sapiens
MTMEKGMSSGEGLPSRSSQVSAGKITAKELETKQSYKEKRGGFVLVHAGAGYHSESKAKEYKHVCKRACQKAIEKLQAGALATDAVTAALVELEDSPFTNAGMGSNLNLLGEIECDASIMDGKSLNFGAVGALSGIKNPVSVANRLLCEGQKGKLSAGRIPPCFLVGEGAYRWAVDHGIPSCPPNIMTTRFSLAAFKRNKRKLELAERVDTDFMQLKKRRQSSEKENDSGTLDTVGAVVVDHEGNVAAAVSSGGLALKHPGRVGQAALYGCGCWAENTGAHNPYSTAVSTSGCGEHLVRTILARECSHALQAEDAHQALLETMQNKFISSPFLASEDGVLGGVIVLRSCRCSAEPDSSQNKQTLLVEFLWSHTTESMCVGYMSAQDGKAKTHISRLPPGAVAGQSVAIEGGVCRLESPVN

SEQ ID NO: 13 - protein, 381 aa, Homo sapiens
KIKDCYGLGSGQNHFIKDSQWEQQAEIFNASYKKYLDREWEEEPLSTATFYFLLPSCLFAMPPEVKGPSGMACVLGIHWTRSHNFFLYSLNRTLKDKADPEGVWPCAAPIAVSQLSCSSSYLVLACEDGVLTLWDLAKGFPLGVAALPQGCFCQSIHFLKYFSVHKGQNMYPEGQVKSQMKCVVLCTDASLHLVEASGTQGPTISVLVERPVKHLDKTICAVAPVPALPGMVLIFSKNGSVCLMDVAKREIICAFAPPGAFPLEVPWKPVFAVSPDHPCFLLRGDYSHETASTDDAGIQYSVFYFNFEACPLLENISKNCTIPQRDLDNMAFPQALPLEKRCERFLQKSYRKLEKNPEKEEEHWARLQRYSLSLQRENFKK

SEQ ID NO: 14 - protein, 108 aa, Homo sapiens
MGKGYYLKGKIGKVPVRFLVDSGAQVSVVHPNLWEEVTDGDLDTLQPFENVVKVANGAEMKILGVWDTAVSLGKLKLKAQFLVANASAEEAIIGTDLQDHNAILDFEH

SEQ ID NO: 15 - protein, 522 aa, Homo sapiens
MSFICGLQSAARNHVFFRFNSLSNWRKCNTLASTSRGCHQVQVNHIVNKYQGLGVNQCDRWSFLPGNFHFYSTFNNKRTGGLSSTKSKEIWRITSKCTVWNDAFSRQLLIKEVTAVPSLSVLHPLSPASIRAIRNFHTSPRFQAAPVPLLLMILKPVQKLFAIIVGRGIRKWWQALPPNKKEVVKENIRKNKWKLFLGLSSFGLLFVVFYFTHLEVSPITGRSKLLLLGKEQFRLLSELEYEAWMEEFKNDMLTEKDARYLAVKEVLCHLIECNKDVPGISQINWVIHVVDSPIINAFVLPNGQMFVFTGFLNSVTDIHQLSFLLGHEIAHAVLGHAAEKAGMVHLLDFLGMIFLTMIWAICPRDSLALLCQWIQSKLQEYMFNRPYSRKLEAEADKIGLLLAAKACADIRASSVFWQQMEFVDSLHGQPKMPEWLSTHPSHGNRVEYLDRLIPQALKIREMCNCPPLSNPDPRLLFKLSTKHFLEESEKEDLNITKKQKMDTLPIQKQEQIPLTYIVEKRT

SEQ ID NO: 16 - protein, 285 aa, Homo sapiens
MNNLSFSELCCLFCCPPCPGKIASKLAFLPPDPTYTLMCDESGSRWTLHLSERADWQYSSREKDAIECFMTRTSKGNRIACMFVRCSPNAKYTLLFSHGNAVDLGQMSSFYIGLGSRINCNIFSYDYSGYGASSGKPTEKNLYADIEAAWLALRTRYIRPENVIIYGQSIGTVPSVDLAARYESAAVILHSPLTSGMRVAFPDTKKTYCFDAFPNIDKISKITSPVLIIHGTEDEVIDFSHGLALFERCQRPVEPLWVEGAGHNDVELYGQYLERLKQFVSQELV

SEQ ID NO: 17 - protein, 1513 aa, Homo sapiens
GSGCLGAEKREGKNRWQGEASMERLLAQLCGSSAAWPLPLWEGDTTGHCFTQLVLSALPHALLAVLSACYLGTPRSPDYILPCSPGWRLRLAASFLLSVFPLLDLLPVALPPGAGPGPIGLEVLAGCVAAVAWISHSLALWVLAHSPHGHSRGPLALALVALLPAPALVLTVLWHCQRGTLLPPLLPGPMARLCLLILQLAALLAYALGWAAPGGPREPWAQEPLLPEDQEPEVAEDGESWLSRFSYAWLAPLLARGACGELRQPQDICRLPHRLQPTYLARVFQAHWQEGARLWRALYGAFGRCYLALGLLKLVGTMLGFSGPLLLSLLVGFLEEGQEPLSHGLLYALGLAGGAVLGAVLQNQYGYEVYKVTLQARGAVLNILYCKALQLGPSRPPTGEALNLLGTDSERLLNFAGSFHEAWGLPLQLAITLYLLYQQVGVAFVGGLILALLLVPVNKVIATRIMASNQEMLQHKDARVKLVTELLSGIRVIKFCGWEQALGARVEACRARELGRLRVIKYLDAACVYLWAALPVVISIVIFITYVLMGHQLTATKVFTALALVRMLILPLNNFPWVINGLLEAKVSLDRIQLFLDLPNHNPQAYYSPDPPAEPSTVLELHGALFSWDPVGTSLETFISHLEVKKGMLVGIVGKVGCGKSSLLAAIAGELHRLRGHVAVRGLSKGFGLATQEPWIQFATIRDNILFGKTFDAQLYKEVLEACALNDDLSILPAGDQTEVGEKGVTLSGGQRARIALARAVYQEKELYLLDDPLAAVDADVANHLLHRCILGMLSYTTRLLCTHRTEYLERADAVLLMEAGRLIRAGPPSEILPLVQAVPKAWAENGQESDSATAQSVQNPEKTKEGLEEEQSTSGRLLQEESKKEGAVALHVYQAYWKAVGQGLALAILFSLLLMQATRNAADWWLSHWISQLKAENSSQEAQPSTSPASMGLFSPQLLLFSPGNLYIPVFPLPKAAPNGSSDIRFYLTVYATIAGVNSLCTLLRAVLFAAGTLQAAATLHRRLLHRVLMAPVTFFNATPTGRILNRFSSDVACADDSLPFILNILLANAAGLLGLLAVLGSGLPWLLLLLPPLSIMYYHVQRHYRASSRELRRLGSLTLSPLYSHLADTLAGLSVLRATGATYRFEEENLRLLELNQRCQFATSATMQWLDIRLQLMGAAVVSAIAGIALVQHQQGLANPGLVGLSLSYALSLTGLLSGLVSSFTQTEAMLVSVERLEEYTCDLPQEPQGQPLQLGTGWLTQGGVEFQDVVLAYRPGLPNALDGVTFCVQPGEKLGIVGRTGSGKSSLLLVLFRLLEPSSGRVLLDGVDTSQLELAQLRSQLAIIPQEPFLFSGTVRENLDPQGLHKDRALWQALKQCHLSEVITSMGGLDGELGEGGRSLSLGQRQLLCLARALLTDAKILCIDEATASVDQKTDQLLQQTICKRFANKTVLTIAHRLNTILNSDRVLVLQAGRVVELDSPATLRNQPHSLFQQLLQSSQQGVPASLGGP

SEQ ID NO: 18 - protein, 282 aa, Homo sapiens
MAAVRVLVASRLAAASAFTSLSPGGRTPSQRAALHLSVPRPAARVALVLSGCGVYDGTEIHEASAILVHLSRGGAEVQIFAPDVPQMHVIDHTKGQPSEGESRNVLTESARIARGKITDLANLSAANHDAAIFPGGFGAAKNLSTFAVDGKDCKVNKEVERVLKEFHQAGKPIGLCCIAPVLAAKVLRGVEVTVGHEQEEGGKWPYAGTAEAIKALGAKHCVKEVSLRSVLGGFFRNSAHEAHVDQKNKVVTTPAFMCETALHYIHDGIGAMVRKVLELTGK

SEQ ID NO: 19 - protein, 297 aa, Homo sapiens
MAELTALESLIEMGFPRGRAEKALALTGNQGIEAAMDWLMEHEDDPDVDEPLETPLGHILGREPTSSEQGGLEGSGSAAGEGKPALSEEERQEQTKRMLELVAQKQREREEREEREALERERQRRRQGQELSAARQRLQEDEMRRAAEERRREKAEELAARQRVREKIERDKAERAKKYGGSVGSQPPPVAPEPGPVPSSPSQEPPTKREYDQCRIQVRLPDGTSLTQTFRAREQLAAVRLYVELHRGEELGGGQDPVQLLSGFPRRAFSEADMERPLQELGLVPSAVLIVAKKCPS

SEQ ID NO: 20 - protein, 221 aa, Homo sapiens
QVKLKIPFGNKLLDAVCLVPNKSLTYGIILTHGASGDMNLPHLMSLASHLASHGFFCLRFTCKGLNIVHRIKAYKSVLNYLKTSGEYKLAGVFLGGRSMGSRAAASVMCHIEPDDGDDFVRGLICISYPLHHPKQQHKLRDEDLFRLKEPVLFVSGSADEMCEKNLLEKVAQKMQAPHKIHWIEKANHSMAVKGRSTNDVFKEINTQILFWIQEITEMDKK

SEQ ID NO: 21 - protein, 305 aa, Homo sapiens
MNGLSLSELCCLFCCPPCPGRIAAKLAFLPPEATYSLVTEPEPGPGGAGAAPLGTLRASSGAPGRWKLHLTERADFQYSQRELDTIEVFPTKSARRNRVSCMYVRCVTGARYTVLFSHSNAVDLGQMSSFYIGLGSRLHCNIFSYDYSGYGASSGRPSERNLYADIDAAWQALRTRYGISPDSIILYGQSIGTVPTVDLASRYECAAVVLHSPLTSGMRVAFPDTKKTYCFDAFPNIEKVSKITSPVLIIHGTEDEVIDFSHGLALYERCPKAVEPLWVEGTRHNDIELYSQYLEGLRRFISQEL

SEQ ID NO: 22 - protein, 243 aa, Homo sapiens
AQGKDQMWYEDALASSHPIILYLHGNAGTRCLFFTLQVLSSLGYHVVTFDYRGWGDSVGTPSERGMTYDALHVFDWIKARSGDNPVYIWGHSLGTGVATNLVRRLCERETPPDALILESPFTNIREEAKSHPFSIYRYFPGFDWFFLDPITSSGIKFANDENVKHISCPLLILHAEDDPVVPFQLGRKLYSIAAPARSFRDFKVQFVPFHSDLGYRHKYIYKSPELPRILREFLGKSEPEHQH

SEQ ID NO: 23 - protein, 233 aa, Homo sapiens
MDASIMDGKDLSAGAVSAVQCIANPIKLARLVMEKTPHCFLTDQGAAQFAAAMGVPEIPGEKLVTERNKKRLEKEKHEKGAQKTDCQKNLGTVGAVALDCKGNVAYATSTGGIVNKMVGRVGDSPCLAGAGGYADNDIGAVSTTGHGESILKVNLARLTLFHIEQGKTVEEAADLSLGYMKSRVKGLGGLIVVSKTGDWVAKWTSTSMPWAAAKDGKLHFGIDPDDTTITDLP

SEQ ID NO: 24 - protein, 166 aa, Homo sapiens
MLRGVLGKTFRLVGYTIQYGCIAHCAFEYVGGVVMCSGPSMEPTIQNSDIVFAENLSRHFYGIQRGDIVIAKSPSDPKSNICKRVIGLEGDKILTTSPSDFFKSHSYVPMGHVWLEGDNLQNSTDSRCYGPIPYGLIRGRIFFKIWPLSDFGFLRASPNGHRFSDD

SEQ ID NO: 25 - protein, 1091 aa, Homo sapiens
MMDSPKIGNGLPVIGPGTDIGISSLHMVGYLGKNFDSAKVPSDEYCPACREKGKLKALKTYRISFQESIFLCEDLQCIYPLGSKSLNNLISPDLEECHTPHKPQKRKSLESSYKDSLLLANSKKTRNYIAIDGGKVLNSKHNGEVYDETSSNLPDSSGQQNPIRTADSLERNEILEADTVDMATTKDPATVDVSGTGRPSPQNEGCTSKLEMPLESKCTSFPQALCVQWKNAYALCWLDCILSALVHSEELKNTVTGLCSKEESIFWRLLTKYNQANTLLYTSQLSGVKDGDCKKLTSEIFAEIETCLNEVRDEIFISLQPQLRCTLGDMESPVFAFPLLLKLETHIEKLFLYSFSWDFECSQCGHQYQNRHMKSLVTFTNVIPEWHPLNAAHFGPCNNCNSKSQIRKMVLEKVSPIFMLHFVEGLPQNDLQHYAFHFEGCLYQITSVIQYRANNHFITWILDADGNWLECDDLKGPCSERHKKFEVPASEIHIVIWERKISQVTDKEAACLPLKKTNDQHALSNEKPVSLTSCSVGDAASAETASVTHPKDISVAPRTLSQDTAVTHGDHLLSGPKGLVDNILPLTLEETIQKTASVSQLNSEAFLLENKPVAENTGILKTNTLLSQESLMASSVSAPCNEKLIQDQFVDISFPSQVVNTNMQSVQLNTEDTVNTKSVNNTDATGLIQGVKSVEIEKDAQLKQFLTPKTEQLKPERVTSQVSNLKKKETTADSQTTTSKSLQNQSLKENQKKPFVGSWVKGLISRGASFMPLCVSAHNRNTITDLQPSVKGVNNFGGFKTKGINQKASHVSKKARKSASKPPPISKPPAGPPSSNGTAAHPHAHAASEVLEKSGSTSCGAQLNHSSYGNGISSANHEDLVEGQIHKLRLKLRKKLKAEKKKLAALMSSPQSRTVRSENLEQVPQDGSPNDCESIEDLNELPYPIDIASESACTTVPGVSLYSSQTHEEILAELLSPTPVSTELSENGEGDFRYLGMGDSHIPPPVPSEFNDVSQNTHLRQDHNYCSPTKKNPCEVQPDSLTNNACVRTLNLESPMKTDIFDEFFSSSALNALANDTLDLPHFDEYLFENY

SEQ ID NO: 26 - protein, 439 aa, Homo sapiens
MLTGVTDGIFCCLLGTPPNAVGPLESVESSDGYTFVEVKPGRVLRVKHAGPAPAAAPPPPSSASSDAAQGDLSGLVRCQRRITVYRNGRLLVENLGRAPRADLLHGQNGSGEPPAALEVELADPAGSDGRLAPGSAGSGSGSGSGGRRRRARRPKRTIHIDCEKRITSCKGAQADVVLFFIHGVGGSLAIWKEQLDFFVRLGYEVVAPDLAGHGASSAPQVAAAYTFYALAEDMRAIFKRYAKKRNVLIGHSYGVSFCTFLAHEYPDLVHKVIMINGGGPTALEPSFCSIFNMPTCVLHCLSPCLAWSFLKAGFARQGAKEKQLLKEGNAFNVSSFVLRAMMSGQYWPEGDEVYHAELTVPVLLVHGMHDKFVPVEEDQRMAEILLLAFLKLIDEGSHMVMLECPETVNTLLHEFLLWEPEPSPKALPEPLPAPPEDKK

SEQ ID NO: 27 - protein, 514 aa, Homo sapiens
MRRQWGSAMRAAEQAGCMVSASRAGQPEAGPWSCSGVILSRSPGLVLCHGGIFVPFLRAGSEVLTAGAVFLPGDSCRDDLRLHVQWAPTGAPRGQARTGPPRLARPMRDLSPPPPSQASLSQSCDWRITETLGWFALLGVRLGQEEWRRRGPMAVSPLGAVPKGAPLLVCGSPFGAFCPDIFLNTLSCGVLSNVAGPLLLTDARCLPGTEGGGVFTARPAGALVALVVAPLCWKAGEWVGFTLLCAAAPLFRAARDALHRLPHSTAALAALLPPEVGVPWGLPLRDSGPLWAAAAVLVECGTVWGSGVAVAPRLVVTCRHVSPREAARVLVRSTTPKSVAIWGRVVFATQETCPYDIAVVSLEEDLDDVPIPVPAEHFHEGEAVSVVGFGVFGQSCGPSVTSGILSAVVQVNGTPVMLQTTCAVHSGSSGGPLFSNHSGNLLGIITSNTRDNNTGATYPHLNFSIPITVLQPALQQYSQTQDLGGLRELDRAAEPVRVVWRLQRPLAEAPRSKL

SEQ ID NO: 28 - protein, 305 aa, Homo sapiens
MAVARLAAVAAWVPCRSWGWAAVPFGPHRGLSVLLARIPQRAPRWLPACRQKTSLSFLNRPDLPNLAYKKLKGKSPGIIFIPGYLSYMNGTKALAIEEFCKSLGHACIRFDYSGVGSSDGNSEESTLGKWRKDVLSIIDDLADGPQILVGSSLGGWLMLHAAIARPEKVVALIGVATADTLVTKFNQLPVELKKEVEMKGVWSMPSKYSEEGVYNVQYSFIKEAEHHCLLHSPIPVNCPIRLLHGMKDDIVPWHTSMQVADRVLSTDVDVILRKHSDHRMREKADIQLLVYTIDDLIDKLSTIVN

SEQ ID NO: 29 - protein, 728 aa, Homo sapiens
QQGSITLSLWTLPDVLIIHLKRFRQEGDRRMKLQNMVKFPLTGLDMTPHVVKRSQSSWSLPSHWSPWRRPYGLGRDPEDYIYDLYAVCNHHGTMQGGHYTAYCKNSVDGLWYCFDDSDVQQLSEDEVCTQTAYILFYQRRTAIPSWSANSSVAGSTSSSLCEHWVSRLPGSKPASVTSAASSRRTSLASLSESVEMTGERSEDDGGCFSTRPFVRSVQRQSLSSRSSVTSPLAVNENCMRPSWSLSAKLQMRSNSPSRFSGDSPIHSSASTLEKIGEAADDKVSISCFGSLRNLSSSYQEPSDSHSLREHKAVGRAPLAVMEGVFKDESDTRRLNSSVVDTQSKHSAQGDRLPPLSGPFDNNNQIAYVDQSDSVDSSPVKEVKAPSHPGSLAKKPESTTKRSPSSKGTSEPEKSLRKGRPALASQESSLSSTSPSSPLPVKVSLKPSRSRSKADSSSRGSGRHSSPAPAQPKKESSPKSQDSVSSPSPQKQKSASALTYTASSTSAKKASGPATRSPFPPGKSRTSDHSLSREGSRQSLGSDRASATSTSKPNSPRVSQARAGEGRGAGKHVRSSSMASLRSPSTSIKSGLKRDSKSEDKGLSFFKSALRQKETRRSTDLGKTALLSKKAGGSSVKSVCKNTGDDEAERGHQPPASQQPNANTTGKEQLVTKDPASAKHSLLSARKSKSSQLDSGVPSSPGGRQSAEKSSKKLSSSMQTSARPSQKPQ

SEQ ID NO: 30 - protein, 561 aa, Homo sapiens
MCGIWALFGSDDCLSVQCLSAMKIAHRGPDAFRFENVNGYTNCCFGFHRLAVVDPLFGMQPIRVKKYPYLWLCYNGEIYNHKKMQQHFEFEYQTKVDGEIILHLYDKGGIEQTICMLDGVFAFVLLDTANKKVFLGRDTYGVRPLFKAMTEDGFLAVCSEAKGLVTLKHSATPFLKVEPFLPGHYEVLDLKPNGKVASVEMVKYHHCRDVPLHALYDNVEKLFPGFEIETVKNNLRILFNNAVKKRLMTDRRIGCLLSGGLDSSLVAATLLKQLKEAQVQYPLQTFAIGMEDSPDLLAARKVADHIGSEHYEVLFNSEEGIQALDEVIFSLETYDITTVRASVGMYLISKYIRKNTDSVVIFSGEGSDELTQGYIYFHKAPSPEKAEEESERLLRELYLFDVLRADRTTAAHGLELRVPFLDHRFSSYYLSLPPEMRIPKNGIEKHLLRETFEDSNLIPKEILWRPKEAFSDGITSVKNSWFKILQEYVEHQVDDAMMANAAQKFPFNTPKTKEGYYYRQVFERHYPGRADWLSHYWMPKWINATDPSARTLTHYKSAVKA

SEQ ID NO: 31 - protein, 830 aa, Homo sapiens
QANCQIAILYQRFQRVVFGISQLLCFSALISELTNQKEVAAWTYHYSTKAYSWNISRKYCQNRYTDLVAIQNKNEIDYLNKVLPYYSSYYWIGIRKNNKTWTWVGTKKALTNEAENWADNEPNNKRNNEDCVEIYIKSPSAPGKWNDEHCLKKKHALCYTASCQDMSCSKQGECLETIGNYTCSCYPGFYGPECEYVRECGELELPQHVLMNCSHPLGNFSFNSQCSFHCTDGYQVNGPSKLECLASGIWTNKPPQCLAAQCPPLKIPERGNMTCLHSAKAFQHQSSCSFSCEEGFALVGPEVVQCTASGVWTAPAPVCKAVQCQHLEAPSEGTMDCVHPLTAFAYGSSCKFECQPGYRVRGLDMLRCIDSGHWSAPLPTCEAISCEPLESPVHGSMDCSPSLRAFQYDTNCSFRCAEGFMLRGADIVRCDNLGQWTAPAPVCQALQCQDLPVPNEARVNCSHPFGAFRYQSVCSFTCNEGLLLVGASVLQCLATGNWNSVPPECQAIPCTPLLSPQNGTMTCVQPLGSSSYKSTCQFICDEGYSLSGPERLDCTRSGRWTDSPPMCEAIKCPELFAPEQGSLDCSDTRGEFNVGSTCHFSCDNGFKLEGPNNVECTTSGRWSATPPTCKGIASLPTPGVQCPALTTPGQGTMYCRHHPGTFGFNTTCYFGCNAGFTLIGDSTLSCRPSGQWTAVTPACRAVKCSELHVNKPIAMNCSNLWGNFSYGSICSFHCLEGQLLNGSAQTACQENGHWSTTVPTCQAGPLTIQEALTYFGGAVASTIGLIMGGTLLALLRKRFRQKDDGKCPLNPHSHLGTYGVFTNAAFDPSP

SEQ ID NO: 32 - protein, 2386 aa, Homo sapiens
MLRGPGPGLLLLAVQCLGTAVPSTGASKSKRQAQQMVQPQSPVAVSQSKPGCYDNGKHYQINQQWERTYLGNALVCTCYGGSRGFNCESKPEAEETCFDKYTGNTYRVGDTYERPKDSMIWDCTCIGAGRGRISCTIANRCHEGGQSYKIGDTWRRPHETGGYMLECVCLGNGKGEWTCKPIAEKCFDHAAGTSYVVGETWEKPYQGWMMVDCTCLGEGSGRITCTSRNRCNDQDTRTSYRIGDTWSKKDNRGNLLQCICTGNGRGEWKCERHTSVQTTSSGSGPFTDVRAAVYQPQPHPQPPPYGHCVTDSGVVYSVGMQWLKTQGNKQMLCTCLGNGVSCQETAVTQTYGGNSNGEPCVLPFTYNGRTFYSCTTEGRQDGHLWCSTTSNYEQDQKYSFCTDHTVLVQTRGGNSNGALCHFPFLYNNHNYTDCTSEGRRDNMKWCGTTQNYDADQKFGFCPMAAHEEICTTNEGVMYRIGDQWDKQHDMGHMMRCTCVGNGRGEWTCIAYSQLRDQCIVDDITYNVNDTFHKRHEEGHMLNCTCFGQGRGRWKCDPVDQCQDSETGTFYQIGDSWEKYVHGVRYQCYCYGRGIGEWHCQPLQTYPSSSGPVEVFITETPSQPNSHPIQWNAPQPSHISKYILRWRPKNSVGRWKEATIPGHLNSYTIKGLKPGVVYEGQLISIQQYGHQEVTRFDFTTTSTSTPVTSNTVTGETTPFSPLVATSESVTEITASSFVVSWVSASDTVSGFRVEYELSEEGDEPQYLDLPSTATSVNIPDLLPGRKYIVNVYQISEDGEQSLILSTSQTTAPDAPPDTTVDQVDDTSIVVRWSRPQAPITGYRIVYSPSVEGSSTELNLPETANSVTLSDLQPGVQYNITIYAVEENQESTPVVIQQETTGTPRSDTVPSPRDLQFVEVTDVKVTIMWTPPESAVTGYRVDVIPVNLPGEHGQRLPISRNTFAEVTGLSPGVTYYFKVFAVSHGRESKPLTAQQTTKLDAPTNLQFVNETDSTVLVRWTPPRAQITGYRLTVGLTRRGQPRQYNVGPSVSKYPLRNLQPASEYTVSLVAIKGNQESPKATGVFTTLQPGSSIPPYNTEVTETTIVITWTPAPRIGFKLGVRPSQGGEAPREVTSDSGSIVVSGLTPGVEYVYTIQVLRDGQERDAPIVNKVVTPLSPPTNLHLEANPDTGVLTVSWERSTTPDITGYRITTTPTNGQQGNSLEEVVHADQSSCTFDNLSPGLEYNVSVYTVKDDKESVPISDTIIPAVPPPTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSQMQVTDVQDNSISVKWLPSSSPVTGYRVTTTPKNGPGPTKTKTAGPDQTEMTIEGLQPTVEYVVSVYAQNPSGESQPLVQTAVTNIDRPKGLAFTDVDVDSIKIAWESPQGQVSRYRVTYSSPEDGIHELFPAPDGEEDTAELQGLRPGSEYTVSVVALHDDMESQPLIGTQSTAIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTDELPQLVTLPHPNLHGPEILDVPSTVQKTPFVTHPGYDTGNGIQLPGTSGQQPSVGQQMIFEEHGFRRTTPPTTATPIRHRPRPYPPNVGEEIQIGHIPREDVDYHLYPHGPGLNPNASTGQEALSQTTISWAPFQDTSEYIISCHPVGTDEEPLQFRVPGTSTSATLTGLTRGATYNVIVEALKDQQRHKVREEVVTVGNSVNEGLNQPTDDSCFDPYTVSHYAVGDEWERMSESGFKLLCQCLGFGSGHFRCDSSRWCHDNGVNYKIGEKWDRQGENGQMMSCTCLGNGKGEFKCDPHEATCYDDGKTYHVGEQWQKEYLGAICSCTCFGGQRGWRCDNCRRPGGEPSPEGTTGQSYNQYSQRYHQRTNTNVNCPIECFMPLDVQADREDSRE

SEQ ID NO: 33 - protein, 953 aa, Homo sapiens
RYNKNLEEAKRIGIKKAITANISIGAAFLLIYASYALAFWYGTTLVLSGEYSIGQVLTVFFSVLIGAFSVGQASPSIEAFANARGAAYEIFKIIDNKPSIDSYSKSGHKPDNIKGNLEFRNVHFSYPSRKEVKILKGLNLKVQSGQTVALVGNSGCGKSTTVQLMQRLYDPTEGMVSVDGQDIRTINVRFLREIIGVVSQEPVLFATTIAENIRYGRENVTMDEIEKAVKEANAYDFIMKLPHKFDTLVGERGAQLSGGQKQRIAIARALVRNPKILLLDEATSALDTESEAVVQVALDKARKGRTTIVIAHRLSTVRNADVIAGFDDGVIVEKGNHDELMKEKGIYFKLVTMQDESIPPVSFWRIMKLNLTEWPYFVVGVFCAIINGGLQPAFAIIFSKIIGVFTRIDDPETKRQNSNLFSLLFLALGIISFITFFLQGFTFGKAGEILTKRLRYMVFRSMLRQDVSWFDDPKNTTGALTTRLANDAAQVKGAIGSRLAVITQNIANLGTGIIISFIYGWQLTLLLLAIVPIIAIAGVVEMKMLSGQALKDKKELEGSGKIATEAIENFRTVVSLTQEQKFEHMYAQSLQVPYRNSLRKAHIFGITFSFTQAMMYFSYAGCFRFGAYLVAHKLMSFEDVLLVFSAVVFGAMAVGQVSSFAPDYAKAKISAAHIIMIIEKTPLIDSYSTEGLMPNTLEGNVTFGEVVFNYPTRPDIPVLQGLSLEVKKGQTLALVGSSGCGKSTVVQLLERFYDPLAGKVLICELFQLLDGKEIKRLNVQWLRAHLGIVSQEPILFDCSIAENIAYGDNSRVVSQEEIVRAAKEANIHAFIESLPNKYSTKVGDKGTQLSGGQKQRIAIARALVRQPHILLLDEATSALDTESEKVVQEALDKAREGRTCIVIAHRLSTIQNADLIVVFQNGRVKEHGTHQQLLAQKGIYFSMVSVQAGTKRQ

SEQ ID NO: 34 - protein, 1537 aa, Homo sapiens
MSLSFCGNNISSYNINDGVLQNSCFVDALNLVPHVFLLFITFPILFIGWGSQSSKVQIHHNTWLHFPGHNLRWILTFALLFVHVCEIAEGIVSDSRRESRHLHLFMPAVMGFVATTTSIVYYHNIETSNFPKLLLALFLYWVMAFITKTIKLVKYCQSGLDISNLRFCITGMMVILNGLLMAVEINVIRVRRYVFFMNPQKVKPPEDLQDLGVRFLQPFVNLLSKATYWWMNTLIISAHKKPIDLKAIGKLPIAMRAVTNYVCLKDAYEEQKKKVADHPNRTPSIWLAMYRAFGRPILLSSTFRYLADLLGFAGPLCISGIVQRVNETQNGTNNTTGISETLSSKEFLENAYVLAVLLFLALILQRTFLQASYYVTIETGINLRGALLAMIYNKILRLSTSNLSMGEMTLGQINNLVAIETNQLMWFLFLCPNLWAMPVQIIMGVILLYNLLGSSALVGAAVIVLLAPIQYFIATKLAEAQKSTLDYSTERLKKTNEILKGIKLLKLYAWEHIFCKSVEETRMKELSSLKTFALYTSLSSKLLTPFFAQTFVTHAYASGNNLKPAEAFASLSLFHILVTPLFLLSTVVRFAVKAIISVQKLNEFLLSDEIGDDSWRTGESSLPFESCKKHTGVQPKTINRKQPGRYHLDSYEQSTRRLRPAETEDIAIKVTNGYFSWGSGLATLSNIDIRIPTGQLTMIVGQVGCGKSSLLLAILGEMQTLEGKVHWSKYVYFYRNRYSVAYAAQKPWLLNATVEENITFGSPFNKQRYKAVTDACSLQPDIDLLPFGDQTEIGERGINLSGGQRQRICVARALYQNTNIVFLDDPFSALDIHLSDHLMQEGILKFLQDDKRTLVLVTHKLQYLTHADWIIAMKDGSVLREGTLKDIQTKDVELYEHWKTLMNRQDQELEKDMEADQTTLERKTLRRAMYSREAKAQMEDEDEEEEEEEDEDDNMSTVMRLRTKMPWKTCWRYLTSGGFFLLILMIFSKLLKHSVIVAIDYWLATWTSEYSINNTGKADQTYYVAGFSILCGAGIFLCLVTSLTVEWMGLTAAKNLHHNLLNKIILGPIRFFDTTPLGLILNRFSADTNIIDQHIPPTLESLTRSTLLCLSAIGMISYATPVFLVALLPLGVAFYFIQKYFRVASKDLQELDDSTQLPLLCHFSETAEGLTTIRAFRHETRFKQRMLELTDTNNIAYLFLSAANRWLEVRTDYLGACIVLTASIASISGSSNSGLVGLGLLYALTITNYLNWVVRNLADLEVQMGAVKKVNSFLTMESENYEGTMDPSQVPEHWPQEGEIKIHDLCVRYENNLKPVLKHVKAYIKPGQKVGICGRTGSGKSSLSLAFFRMVDIFDGKIVIDGIDISKLPLHTLRSRLSIILQDPILFSGSIRFNLDPECKCTDDRLWEALEIAQLKNMVKSLPGGLDAVVTEGGENFSVGQRQLFCLARAFVRKSSILIMDEATASIDMATENILQKVVMTAFADRTVVTIAHRVHTILTADLVIVMKRGNILEYDTPESLLAQENGVFASFVRADM

SEQ ID NO: 35 - protein, 676 aa, Homo sapiens
RAELVALTAVQSEQGEAGGGGSPRRLGLLGSPLPPGAPLPGPGSGSGSACGQRSSAAHKRYRRLQNWGYNVLERPRGWAFVYHVFIFLLVFSCLVLSVLSTIQEHQELANECLLILEFVMIVVFGLEYIVRVWSAGCCCRYRGWQGRFRFARKPFCVIDFIVFVASVAVIAAGTQGNIFATSALRSMRFLQILRMVRMDRRGGTWKLLGSVVYAHSKELITAWYIGFLVLIFASFLVYLAEKDANSDFSSYADSLWWGTITLTTIGYGDKTPHTWLGRVLAAGFALLGISFFALPAGILGSGFALKVQEQHRQKHFEKRRMPAANLIQAAWRLYSTDMSRAYLTATWYYYDSILPSFRELALLFEHVQRARNGGLRPLEVRRAPVPDGAPSRYPPVATCHRPGSTSFCPGESSRMGIKDRIRMGSSQRRTGPSKQHLAPPTMPTSPSSEQVGEATSPTKVQKSWSFNDRTRFRASLRLKPRTSAEDAPSEEVAEEKSYQCELTVDDIMPAVKTVIRSIRILKFLVAKRKFKETLRPYDVKDVIEQYSAGHLDMLGRIKSLQTRVDQIVGRGPGDRKAREKGDKGPSDAEVVDEISMMGRVVKVEKQVQSIEHKLDLLVGFYSRWLRSGTSASLGAVQVPLFDPDITSDYHSPVDHEDISVSAQTLSISRSVSTNMD

SEQ ID NO: 36 - protein, 361 aa, Homo sapiens
QIFPWKCQSTQRDLWNIFKLWGWTMLCCDFLAHHGTDCWTYHYSEKPMNWQRARRFCRDNYTDLVAIQNKAEIEYLEKTLPFSRSYYWIGIRKIGGIWTWVGTNKSLTEEAENWGDGEPNNKKNKEDCVEIYIKRNKDAGKWNDDACHKLKAALCYTASCQPWSCSGHGECVEIINNYTCNCDVGYYGPQCQFVIQCEPLEAPELGTMDCTHPLGNFSFSSQCAFSCSEGTNLTGIEETTCGPFGNWSSPEPTCQVIQCEPLSAPDLGIMNCSHPLASFSFTSACTFICSEGTELIGKKKTICESSGIWSNPSPICQKLDKSFSMIKEGDYNPLFIPVAVMVTAFSGLAFIIWLARRLKKG

SEQ ID NO: 37 - protein, 611 aa, Homo sapiens
QKEGKKERAVVDKVFFSRLIQILKIMVPRTFCKETGYLVLIAVMLVSRTYCDVWMIQNGTLIESGIIGRSRKDFKRYLLNFIAAMPLISLVNNFLKYGLNELKLCFRVRLTKYLYEEYLQAFTYYKMGNLDNRIANPDQLLTQDVEKFCNSVVDLYSNLSKPFLDIVLYIFKLTSAIGAQGPASMMAYLVVSGLFLTRLRRPIGKMTITEQKYEGEYRYVNSRLITNSEEIAFYNGNKREKQTVHSVFRKLVEHLHNFILFRFSMGFIDSIIAKYLATVVGYLVVSRPFLDLSHPRHLKSTHSELLEDYYQSGRMLLRMSQALGRIVLAGREMTRLAGFTARITELMQVLKDLNHGKYERTMVSQQEKGIEGVQVIPLIPGAGEIIIADNIIKFDHVPLATPNGDVLIRDLNFEVRSGANVLICGPNGCGKSSLFRVLGELWPLFGGRLTKPERGKLFYVPQRPYMTLGTLRDQVIYPDGREDQKRKGISDLVLKEYLDNVQLGHILEREGGWDSVQDWMDVLSGGEKQRMAMARLFYHKPQFAILDECTSAVSVDVEGYIYSHCRKVGITLFTVSHRKSLWKHHEYYLHMDGRGNYEFKQITEDTVEFGS

SEQ ID NO: 38 - protein, 522 aa, Homo sapiens [sequence omitted]

SEQ ID NO: 39 - protein, 236 aa, Homo sapiens
LTERADFQYSQRELDTIEVFPTKSARGNRVSCMYVRCVPGARYTVFFSHGNAVDLSQMSSFYIGLGSRLHCNIFYDYSGYGASAGRPSERNLYADIDAAWQALHTRYGISPDSIILYGQSIGTVPTVDLASRYECAAVVLHSPLTSGMRVAFPDTKTYCFDAFPNIEKVSKITSPVLIIHGMEDEVIDFSHGLALYERCPKAVEPLWVEGAGHNDIELYSQYLERLRRFISQELPS

SEQ ID NO: 40 - protein, 4647 aa, Homo sapiens
MSEPGGGGGEDGSAGLEVSAVQNVADVSVLQKHLRKLVPLLLEDGGEAPAALEAALEEKSALEQMRKFLSDPQVHTVLVERSTLKEDVGDEGEEEKEFISYNINIDIHYGVKSNSLAFIKRTPVIDADKPVSSQLRVLTLSEDSPYETLHSFISNAVAPFFKSYIRESGKADRDGDKMAPSVEKKIAELEMGLLHLQQNIEIPEISLPIHPMITNVAKQCYERGEKPKVTDFGDKVEDPTFLNQLQSGVNRWIREIQKVTKLDRDPASGTALQEISFWLNLERALYRIQEKRESPEVLLTLDILKHGKRFHATVSFDTDTGLKQALETVNDYNPLMKDFPLNDLLSATELDKIRQALVAIFTHLRKIRNTKYPIQRALRLVEAISRDLSSQLLKVLGTRKLMHVAYEEFEKVMVACFEVFQTWDDEYEKLQVLLRDIVKRKREENLKMVWRINPAHRKLQARLDQMRKFRRQHEQLRAVIVRVLRPQVTAVAQQNQGEVPEPQDMKVAEVLFDAADANAIEEVNLAYENVKEVDGLDVSKEGTEAWEAAMKRYDERIDRVETRITARLRDQLGTAKNANEMFRIFSRFNALFVRPHIRGAIREYQTQLIQRVKDDIESLHDKFKVQYPQSQACKMSHVRDLPPVSGSIIWAKQIDRQLTAYMKRVEDVLGKGWENHVEGQKLKQDGDSFRMKLNTQEIFDDWARKVQQRNLGVSGRIFTIESTRVRGRTGNVLKLKVNFLPEIITLSKEVRNLKWLGFRVPLAIVNKAHQANQLYPFAISLIESVRTYERTCEKVEERNTISLLVAGLKKEVQALIAEGIALVWESYKLDPYVQRLAETVFNFQEKVDDLLIIEEKIDLEVRSLETCMYDHKTFSEILNRVQKAVDDLNLHSYSNLPIWVNKLDMEIERILGVRLQAGLRAWTQVLLGQAEDKAEVDMDTDAPQVSHKPGGEPKIKNVVHELRITNQVIYLNPPIEECRYKLYQEMFAWKMVVLSLPRIQSQRYQVGVHYELTEEEKFYRNALTRMPDGPVALEESYSAVMGIVSEVEQYVKVWLQYQCLWDMQAENIYNRLGEDLNKWQALLVQIRKARGTFDNAETKKEFGPVVIDYGKVQSKVNLKYDSWHKEVLSKFGQMLGSNMTEFHSQISKSRQELEQHSVDTASTSDAVTFITYVQSLKRKIKQFEKQVELYRNGQRLLEKQRFQFPPSWLYIDNIEGEWGAFNDIMRRKDSAIQQQVANLQMKIVQEDRAVESRTTDLLTDWEKTKPVTGNLRPEEALQALTIYEGKFGRLKDDREKCAKAKEALELTDTGLLSGSEERVQVALEELQDLKGVWSELSKVWEQIDQMKEQPWVSVQPRKLRQNLDALLNQLKSFPARLRQYASYEFVQRLLKGYMKINMLVIELKSEALKDRHWKQLMKRLHVNWVVSELTLGQIWDVDLQKNEAIVKDVLLVAQGEMALEEFLKQAKVWNTYELDLVNYQNKCRLIRGWDDLFNKVKEHINSVSAMKLSPYYKVFEEDALSWEDKLNRIMALFDVWIDVQRRWVYLEGIFTGSADIKHLLPVETQRFQSISTEFLALMKKVSKSPLVMDVLNIQGVQRSLERLADLLGKIQKALGEYLERERSSFPRFYFVGDEDLLEIIGNSKNVAKLQKHFKKMFAGVSSIILNEDNSVVLGISSREGEEVMFKTPVSITEHPKINEWLTLVEKEMRVTLAKLLAESVTEVEIFGKATSIDPNTYITWIDKYQAQLVVLSAQIAWSENVETALSSMGGGGDAAPLHSVLSNVEVTLNVLADSVLMEQPPLRRRKLEHLITELVHQRDVTRSLIKSKIDNAKSFEWLSQMRFYFDPKQTDVLQQLSIQMANAKFNYGFEYLGVQDKLVQTPLTDRCYLTMTQALEARLGGSPFGPAGTGKTESVKALGHQLGRFVLVFNCDETFDFQAMGRIFVGLCQVGAWGCFDEFNRLEERMLSAVSQQVQCIQEALREHSNPNYDKTSAPITCELLNKQVKVSPDMAIFITMNPAYAGRSNLPDNLKKLFRSLAMTKPDRQLIAQVMLYSQGFRTAEVLANKIVPFFKLCDEQLSSQSHYDFGLRALKSVLVSAGNVKRERIQKIKREKEERGEAVDEGEIAENLPEQEILIQSVCETMVPKLVAEDIPLLFSLLSDVFPGVQYHRGEMTALREELKKVCQEMYLTYGDGEEVGGMWVEKVLQLYQITQINHGLMMVGPSGSGKSMAWRVLLKALERLEGVEGVAHIIDPKAISKDHLYGTLDPNTREWTDGLFTHVLRKIIDSVRGELQKRQWIVFDGDVDPEWVENLNSVLDDNKLLTLPNGERLSLPPNVRIMFEVQDLKYATLATVSRCGMVWFSEDVLSTDMIFNNFLARLRSIPLDEGEDEAQRRRKGKEDEGEEAASPMLQIQRDAATIMQPYFTSNGLVTKALEHAFQLEHIMDLTRLRCLGSLFSMLHQACRNVAQYNANHPDFPMQIEQLERYIQRYLVYAILWSLSGDSRLKMRAELGEYIRRITTVPLPTAPNIPIIDYEVSISGEWSPWQAKVPQIEVETHKVAAPDVVVPTLDTVRHEALLYTWLAEHKPLVLCGPPGSGKTMTLFSALRALPDMEVVGLNFSSATTPELLLKTFDHYCEYRRTPNGVVLAPVQLGKWLVLFCDEINLPDMDKYGTQRVISFIRQMVEHGGFYRTSDQTWVKLERIQFVGACNPPTDPGRKPLSHRFLRHVPVVYVDYPGPASLTQIYGTFNRAMLRLIPSLRTYAEPLTAAMVEFYTMSQERFTQDTQPHYIYSPREMTRWVRGIFEALRPLETLPVEGLIRIWAHEALRLFQDRLVEDEERRWTDENIDTVALKHFPNIDREKAMSRPILYSNWLSKDYIPVDQEELRDYVKARLKVFYEEELDVPLVLFNEVLDHVLRIDRIFRQPQGHLLLIGVSGAGKTTLSRFVAWMNGLSVYQIKVHRKYTGEDFDEDLRTVLRRSGCKNEKIAFIMDESNVLDSGFLERMNTLLANGEVPGLFEGDEYATLMTQCKEGAQKEGLMLDSHEELYKWFTSQVIRNLHVVFTMNPSSEGLKDRAATSPALFNRCVLNWFGDWSTEALYQVGKEFTSKMDLEKPNYIVPDYMPVVYDKLPQPPSHREAIVNSCVFVHQTLHQANARLAKRGGRTMAITPRHYLDFINHYANLFHEKRSELEEQQMHLNVGLRKIKETVDQVEELRRDLRIKSQELEVKNAAANDKLKKMVKDQQEAEKKKVMSQEIQEQLHKQQEVIADKQMSVKEDLDKVEPAVIEAQNAVKSIKKQHLVEVRSMANPPAAVKLALESICLLLGESTTDWKQIRSIIMRENFIPTIVNFSAEEISDAIREKMKKNYMSNPSYNYEIVNRASLACGPMVKWAIAQLNYADMLKRVEPLRNELQKLEDDAKDNQQKANEVEQMIRDLEASIARYKEEYAVLISEAQAIKADLAAVEAKVNRSTALLKSLSAERERWEKTSETFKNQMSTIAGDCLLSAAFIAYAGYFDQQMRQNLFTTWSHHLQQANIQFRTDIARTEYLSNADERLRWQASSLPADDLCTENAIMLKRFNRYPLIIDPSGQATEFIMNEYKDRKITRTSFLDDAFRKNLESALRFGNPLLVQDVESYDPVLNPVLNREVRRTGGRVLITLGDQDIDLSPSFVIFLSTRDPTVEFPPDLCSRVTFVNFTVTRSSLQSQCLNEVLKAERPDVDEKRSDLLKLQGEFQLRLRQLEKSLLQALNEVKGRILDDDTIITTLENLKREAAEVTRKVEETDIVMQEVETVSQQYLPLSTACSSIYFTMESLKQIHFLYQYSLQFFLDIYHNVLYENPNLKGVTDHTQRLSIITKDLFQVAFNRVARGMLHQDHITFAMLLARIKLKGTVGEPTYDAEFQHFLRGNEIVLSAGSTPRIQGLTVEQAEAVVRLSCLPAFKDLIAKVQADEQFGIWLDSSSPEQTVPYLWSEETPATPIGQAIHRLLLIQAFRPDRLLAMAHMFVSTNLGESFMSIMEQPLDLTHIVGTEVKPNTPVLMCSVPGYDASGHVEDLAAEQNTQITSIAIGSAEGFNQADKAINTAVKSGRWVMLKNVHLAPGWLMQLEKKLHSLQPHACFRLFLTMEINPKVPVNLLRAGRIFVFEPPPGVKANMLRTFSSIPVSRICKSPNERARLYFLLAWFHAIIQERLRYAPLGWSKKYEFGESDLRSACDTVDTWLDDTAKASGRQNISPDKIPWSALKTLMAQSIYGGRVDNEFDQRLLNTFLERLFTTRSFDSEFKLACKVDGHKDIQMPDGIRREEFVQWVELLPDTQTPSWLGLPNNAERVLLTTQGVDMISKMLKMQMLEDEDDLAYAETEKKTRTDSTSDGRPAWMRTLHTTASNWLHLIPQTLSHLKRTVENIKDPLFRFFEREVKMGAKLLQDVRQDLADVVQVCEGKKKQTNYLRTLINELVKGILPRSWSHYTVPAGMTVIQWVSDFSERIKQLQNISLAAASGGAKELKNIHVCLGGLFVPEAYITATRQYVAQANSWSLEELCLEVNVTTSQGATLDACSFGVTGLKLQGATCNNNKLSLSNAISTALPLTQLRWVKQTNTEKKASVVTLPVYLNFTRADLIFTVDFEIATKEDPRSFYERGVAVLCTE

SEQ ID NO: 41 - protein, 1232 aa, Homo sapiens
MANGVIPPPGGASPLPQVRVPLEEPPLSPDVEEEDDDLGKTLAVSRFGDLISKPPAWDPEKPSRSYSERDFEFHRHTSHHTHHPLSARLPPPHKLRRLPPTSARHTRRKRKKEKTSAPPSEGTPPIQEEGGAGVDEEEEEEEEEEGESEAEPVEPPHSGTPQKAKFSIGSDEDDSPGLPGRAAVTKPLPSVGPHTDKSPQHSSSSPSPRARASRLAGEKSRPWSPSASYDLRERLCPGSALGNPGGPEQQVPTDEAEAQMLGSADLDDMKSHRLEDNPGVRRHLVKKPSRTQGGRGSPSGLAPILRRKKKKKKLDRRPHEVFVELNELMLDRSQEPHWRETARWIKFEEDVEEETERWGKPHVASLSFRSLLELRRTIAHGAALLDLEQTTLPGIAHLVVETMIVSDQIRPEDRASVLRTLLLKHSHPNDDKDSGFFPRNPSSSSMNSVLGNHHPTPSHGPDGAVPTMADDLGEPAPLWPHDPDAKEKPLHMPGGDGHRGKSLKLLEKIPEDAEATVVLVGCVPFLEQPAAAFVRLNEAVLLESVLEVPVPVRFLFVMLGPSHTSTDYHELGRSIATLMSDKLFHEAAYQADDRQDLLSAISEFLDGSIVIPPSEVEGRDLLRSVAAFQRELLRKRREREQTKVEMTTRGGYTAPGKELSLELGGSEATPEDDPLLRTGSVFGGLVRDVRRRYPHYPSDLRDALHSQCVAAVLFIYFAALSPAITFGGLLGEKTEGLMGVSELIVSTAVLGVLFSLLGAQPLLVVGFSGPLLVFEEAFFKFCRAQDLEYLTGRVWVGLWLVVFVLALVAAEGSFLVRYISPFTQEIFAFLISLIFIYETFYKLYKVFTEHPLLPFYPPEGALEGSLDAGLEPNGSALPPTEGPPSPRNQPNTALLSLILMLGTFFIAFFLRKFRNSRFLGGKARRIIGDFGIPISILVMVLVDYSITDTYTQKLTVPTGLSVTSPDKRSWFIPPLGSARPFPPWMMVAAAVPALLVLILIFMETQITALIVSQKARRLLKGSGFHLDLLLIGSLGGLCGLFGLPWLTAATVRSVTHVNALTVMRTAIAPGDKPQIQEVREQRVTGVLIASLVGLSIVMGAVLRRIPLAVLFGIFLYMGVTSLSGIQLSQRLLLILMPAKHHPEQPYVTKVKTWRMHLFTCIQLGCIALLWVVKSTAASLAFPFLLLLTVPLRHCLLPRLFQDRELQALDSEDAEPNFDEDGQDEYNELHMPV

SEQ ID NO: 42 - protein, 1515 aa, Homo sapiens
DWNVTWNTSNPDFTKCFQNTVLVWVPCFYLWACFPFYFLYLSRHDRGYIQMTPLNKTKTALGFLLWIVCWADLFYSFWERSRGIFLAPVFLVSPTLLGITMLLATFLIQLERRKGVQSSGIMLTFWLVALVCALAILRSKIMTALKEDAQVDLFRDITFYVYFSLLLIQLVLSCFSDRSPLFSETIHDPNPCPESSASFLSRITFWWITGLIVRGYRQPLEGSDLWSLNKEDTSEQVVPVLVKNWKKECAKTRKQPVKVVYSSKDPAQPKESSKVDANEEVEALIVKSPQKEWNPSLFKVLYKTFGPYFLMSFFFKAIHDLMMFSGPQILKLLIKFVNDTKAPDWQGYFYTVLLFVTACLQTLVLHQYFHICFVSGMRIKTAVIGAVYRKALVITNSARKSSTVGEIVNLMSVDAQRFMDLATYINMIWSAPLQVILALYLLWLNLGPSVLAGVAVMVLMVPVNAVMAMKTKTYQVAHMKSKDNRIKLMNEILNGIKVLKLYAWELAFKDKVLAIRQEELKVLKKSAYLSAVGTFTWVCTPFLVALCTFAVYVTIDENNILDAQTAFVSLALFNILRFPLNILPMVISSIVQASVSLKRLRIFLSHEELEPDSIERRPVKDGGGTNSITVRNATFTWARSDPPTLNGITFSIPEGALVAVVGQVGCGKSSLLSALLAEMDKVEGHVAIKGSVAYVPQQAWIQNDSLRENILFGCQLEEPYYRSVIQACALLPDLEILPSGDRTEIGEKGVNLSGGQKQRVSLARAVYSNADIYLFDDPLSAVDAHVGKHIFENVIGPKGMLKNKTRILVTHSMSYLPQVDVIIVMSGGKISEMGSYQELLARDGAFAEFLRTYASTEQEQDAEENGVTGVSGPGKEAKQMENGMLVTDSAGKQLQRQLSSSSSYSGDISRHHNSTAELQKAEAKKEETWKLMEADKAQTGQVKLSVYWDYMKAIGLFISFLSIFLFMCNHVSALASNYWLSLWTDDPIVNGTQEHTKVRLSVYGALGISQGIAVFGYSMAVSIGGILASRCLHVDLLHSILRSPMSFFERTPSGNLVNRFSKELDTVDSMIPEVIKMFMGSLFNVIGACIVILLATPIAAIIIPPLGLIYFFVQRFYVASSRQLKRLESVSRSPVYSHFNETLLGVSVIRAFEEQERFIHQSDLKVDENQKAYYPSIVANRWLAVRLECVGNCIVLFAALFAVISRHSLSAGLVGLSVSYSLQVTTYLNWLVRMSSEMETNIVAVERLKEYSETEKEAPWQIQETAPPSSWPQVGRVEFRNYCLRYREDLDFVLRHINVTINGGEKVGIVGRTGAGKSSLTLGLFRINESAEGEIIIDGINIAKIGLHDLRFKITIIPQDPVLFSGSLRMNLDPFSQYSDEEVWTSLELAHLKDFVSALPDKLDHECAEGGENLSVGQRQLVCLARALLRKTKILVLDEATAAVDLETDDLIQSTIRTQFEDCTVLTIAHRLNTIMDYTRVIVLDKGEIQEYGAPSDLLQQRGLFYSMAKDAGLV

SEQ ID NO: 43 - protein, 357 aa, Homo sapiens
FCRAQDLEYLTGRVWVGLWLVVFVLALVAAEGSFLVRYISPFTQEIFAFLISLIFIYETFYKLYKVFTEHPLLPFYPPEPGGVPGCWSGAKWQLPPTEGPPSPRNQPNTALLSLILMLGTFFIAFFLRKFRNSRFLGGKARRIIGDFGIPISILVMVLVDYSITDTYTQKLTVPTGLSVTSPDKRSWFIPPLGSARPFPPWMMVAAAVPALLVLILIFMETQITALIVSQKARRLLKGSGFHLDLLLIGSLGGLCGLFGLPWLTAATVRSVTHVNALTVMRTAIAPGDKPQIQEVREQRVTGVLIASLVGLSIVMGAVLRRIPLAVLFGIFLYMGVTSLSGIQLSQRLLLILMPAKH

SEQ ID NO: 44 - protein, 2147 aa, Homo sapiens
MAFWTQLMLLLWKNFMYRRRQPVQLLVELLWPLFLFFILVAVRHSHPPLEHHECHFPNKPLPSAGTVPWLQGLICNVNNTCFPQLTPGEEPGRLSNFNDSLVSRLLADARTVLGGASAHRTLAGLGKLIATLRAARSTAQPQPTKQSPLEPPMLDVAELLTSLLRTESLGLALGQAQEPLHSLLEAAEDLAQELLALRSLVELRALLQRPRGTSGPLELLSEALCSVRGPSSTVGPSLNWYEASDLMELVGQEPESALPDSSLSPACSELIGALDSHPLSRLLWRRLKPLILGKLLFAPDTPFTRKLMAQVNRTFEELTLLRDVREVWEMLGPRIFTFMNDSSNVAMLQRLLQMQDEGRRQPRPGGRDHMEALRSFLDPGSGGYSWQDAHADVGHLVGTLGRVTECLSLDKLEAAPSEAALVSRALQLLAEHRFWAGVVFLGPEDSSDPTEHPTPDLGPGHVRIKIRMDIDVVTRTNKIRDRFWDPGPAADPLTDLRYVWGGFVYLQDLVERAAVRVLSGANPRAGLYLQQMPYPCYVDDVFLRVLSRSLPLFLTLAWIYSVTLTVKAVVREKETRLRDTMRAMGLSRAVLWLGWFLSCLGPFLLSAALLVLVLKLGDILPYSHPGVVFLFLAAFAVATVTQSFLLSAFFSRANLAAACGGLAYFSLYLPYVLCVAWRDRLPAGGRVAASLLSPVAFGFGCESLALLEEQGEGAQWHNVGTRPTADVFSLAQVSGLLLLDAALYGLATWYLEAVCPGQYGIPEPWNFPFRRSYWCGPRPPKSPAPCPTPLDPKVLVEEAPPGLSPGVSVRSLEKRFPGSPQPALRGLSLDFYQGHITAFLGHNGAGKTTTLSILSGLFPPSGGSAFILGHDVRSSMAAIRPHLGVCPQYNVLFDMLTVDEHVWFYGRLKGLSAAVVGPEQDRLLQDVGLVSKQSVQTRHLSGGMQRKLSVAIAFVGGSQVVILDEPTAGVDPASRRGIWELLLKYREGRTLILSTHHLDEAELLGDRVAVVAGGRLCCCGSPLFLRRHLGSGYYLTLVKARLPLTTNEKADTDMEGSVDTRQEKKNGSQGSRVGTPQLLALVQHWVPGARLVEELPHELVLVLPYTGAHDGSFATLFRELDTRLAELRLTGYGISDTSLEEIFLKVVEECAADTDMEDGSCGQHLCTGIAGLDVTLRLKMPPQETALENGEPAGSAPETDQGSGPDAVGRVQGWALTRQQLQALLLKRFLLARRSRRGLFAQIVLPALFVGLALVFSLIVPPFGHYPALRLSPTMYGAQVSFFSEDAPGDPGRARLLEALLQEAGLEEPPVQHSSHRFSAPEVPAEVAKVLASGNWTPESPSPACQCSRPGARRLLPDCPAAAGGPPPPQAVTGSGEVVQNLTGRNLSDFLVKTYPRLVRQGLKTKKWVNEVRYGGFSLGGRDPGLPSGQELGRSVEELWALLSPLPGGALDRVLKNLTAWAHSLDAQDSLKIWFNNKGWHSMVAFVNRASNAILRAHLPPGPARHAHSITTLNHPLNLTKEQLSEGALMASSVDVLVSICVVFAMSFVPASFTLVLIEERVTRAKHLQLMGGLSPTLYWLGNFLWDMCNYLVPACIVVLIFLAFQQRAYVAPANLPALLLLLLLYGWSITPLMYPASFFFSVPSTAYVVLTCINLFIGINGSMATFVLELFSDQQKLQEVSRILKQVFLIFPHFCLGRGLIDMVRNQAMADAFERLGDRQFQSPLRWEVVGKNLLAMVIQGPLFLLFTLLLQHRSQLLPQPRVRSLPLLGEEDEDVARERERVVQGATQGDVLVLRNLTKVYRGQRMPAVDRLCLGIPPGECFGLLGVNGAGKTSTFRMVTGDTLASRGEAVLAGHSVAREPSAAHLSMGYCPQSDAIFELLTGREHLELLARLRGVPEAQVAQTAGSGLARLGLSWYADRPAGTYSGGNKRKLATALALVGDPAVVFLDEPTTGMDPSARRFLWNSLLAVVREGRSVMLTSHSMEECEALCSRLAIMVNGRFRCLGSPQHLKGRFAAGHTLTLRVPAARSQPAAAFVAAEFPGAELREAHGGRLRFQLPPGGRCALARVFGELAVHGAEHGVEDFSVSQTMLEEVFLYFSKDQGKDEDTEEQKEAGVGVDPAPGLQHPKRVSQFLDDPSTAETVL

SEQ ID NO: 45 - protein, 650 aa, Homo sapiens
MRLKNLTFIIILIISGELYAEEKPCGFPHVENGRIAQYYYTFKSFYFPMSIDKKLSFFCLAGYTTESGRQEEQTTCTTEGWSPEPRCFKKCTKPDLSNGYISDVKLLYKIQENMHYGCASGYKTTGGKDEEVVQCLSDGWSSQPTCRKEHETCLAPELYNGNYSTTQKTFKVKDKVQYECATGYYTAGGKKTEEVECLTYGWSLTPKCTKLKCSSLRLIENGYFHPVKQTYEEGDVVQFFCHENYYLSGSDLIQCYNFGWYPESPVCEGRRNRCPPPPLPINSKIQTHSTTYRHGEIVHIECELNFEIHGSAEIRCEDGKSTEPPKCIEGQEKVACEEPPFIENGAANLHSKIYYNGDKVTYACKSGYLLHGSNEITCNRGKWTLPPECVENNENCKHPPVVMNGAVADGILASYATGSSVEYRCNEYYLLRGSKISRCEQGKWSSPPVCLEPCTVNVDYMNRNNIEMKWKYEGKVLHGDLIDFVCKQGYDLSPLTPLSELSVQCNRGEVKYPLCTRKESKGMCTSPPLIKHGVIISSTVDTYENGSSVEYRCFDHHFLEGSREAYCLDGMWTTPPLCLEPCTLSFTEMEKNNLLLKWDFDNRPHILHGEYIEFICRGDTYPAELYITGSILRMQCDRGQLKYPRCIPRQ

SEQ ID NO: 46 - protein, 2496 aa, Homo sapiens
MGAAAGRSPHLGPAPARRPQRSLLLLQLLLLVAAPGSTQAQAAPFPELCSYTWEAVDTKNNVLYKINICGSVDIVQCGPSSAVCMHDLKTRTYHSVGDSVLRSATRSLLEFNTTVSCDQQGTNHRVQSSIAFLCGKTLGTPEFVTATECVHYFEWRTTAACKKDIFKANKEVPCYVFDEELRKHDLNPLIKLSGAYLVDDSDPDTSLFINVCRDIDTLRDPGSQLRACPPGTAACLVRGHQAFDVGQPRDGLKLVRKDRLVLSYVREEAGKLDFCDGHSPAVTITFVCPSERREGTIPKLTAKSNCRYEIEWITEYACHRDYLESKTCSLSGEQQDVSIDLTPLAQSGGSSYISDGKEYLFYLNVCGETEIQFCNKKQAAVCQVKKSDTSQVKAAGRYHNQTLRYSDGDLTLIYFGGDECSSGFQRMSVINFECNKTAGNDGKGTPVFTGEVDCTYFFTWDTEYACVKEKEDLLCGATDGKKRYDLSALVRHAEPEQNWEAVDGSQTETEKKHFFINICHRVLQEGKARGCPEDAAVCAVDKNGSKNLGKFISSPMKEKGNIQLSYSDGDDCGHGKKIKTNITLVCKPGDLESAPVLRTSGEGGCFYEFEWHTAAACVLSKTEGENCTVFDSQAGFSFDLSPLTKKNGAYKVETKKYDFYINVCGPVSVSPCQPDSGACQVAKRQVASHDEKTWNLGLSNAKLSYYDGMIQLNYRGGTPYNNERHTPRATLITFLCDRDAGVGFPEYQEEDNSTYNFRWYTSYACPEEPLECVVTDPSTLEQYDLSSLAKSEGGLGGNWYAMDNSGEHVTWRKYYINVCRPLNPVPGCNRYASACQMKYEKDQGSFTEVVSISNLGMAKTGPVVEDSGSLLLEYVNGSACTTSDGRQTTYTTRIHLVCSRGRLNSHPIFSLNWECVVSFLWNTEAACPIQTTTDTDQACSIRDPNSGFVFNLNPLNSSQGYNVSGIGKIFMFNVCGTMPVCGTILGKPASGCEAETQTEELKNWKPARPVGIEKSLQLSTEGFITLTYKGPLSAKGTADAFIVRFVCNDDVYSGPLKFLHQDIDSGQGIRNTYFEFETALACVPSPVDCQVTDLAGNEYDLTGLSTVRKPWTAVDTSVDGRKRTFYLSVCNPLPYIPGCQGDAVGSCLVSEGNSWNLGVVQMSPQAAANGSLSIMYVNGDKCGNQRFSTRITFECAQISGSPAFQLQDGCEYVFIWRTVEACPVVRVEGDNCEVKDPRHGNLYDLKPLGLNDTIVSAGEYTYYFRVCGKLSSDVCPTSDKSKVVSSCQEKREPQGFHKVAGLLTQKLTYENGLLKMNFTGGDTCHKVYQRSTAIFFYCDRGTQRPVFLKETSDCSYLFEWRTQYACPPFDLTECSFKDGAGNSFDLSSLSRYSDNWEAITGTGDPEHYLINVCKSLAPQAGTEPCPPEAAACLLGGSKPVNLGRVRDGPQWRDGIIVLKYVDGDLCPDGIRKKSTTIRFTCSESQVNSRPMFISAVEDCEYTFAWPTATACPMKSNEHDDCQVTNPSTGHLFDLSSLSGRAGFTAAYSEKGLVYMSICGENENCPPGVGACFGQTRISVGKANKRLRYVDQVLQLVYKDGSPCPSKSGLSYKSVISFVCRPEARPTNRPMLISLDKQTCTLFFSWHTPLACEQATECSVRNGSSIVDLSPLIHRTGGYEAYDESEDDASDTNPDFYINICQPLNPMHGVPCPAGAAVCKVPIDGPPIDIGRVAGPPILNPIANEIYLNFESSTPCLADKHFNYTSLIAFHCKRGVSMGTPKLLRTSECDFVFEWETPVVCPDEVRMDGCTLTDEQLLYSFNLSSLSTSTFKVTRDSRTYSVGVCTFAVGPEQGGCKDGGVCLLSGTKGASFGRLQSMKLDYRHQDEAVVLSYVNGDRCPPETDDGVPCVFPFIFNGKSYEECIIESRAKLWCSTTADYDRDHEWGFCRHSNSYRTSSIIFKCDEDEDIGRPQVFSEVRGCDVTFEWKTKVVCPPKKLECKFVQKHKTYDLRLLSSLTGSWSLVHNGVSYYINLCQKIYKGPLGCSERASICRRTTTGDVQVLGLVHTQKLGVIGDKVVVTYSKGYPCGGNKTASSVIELTCTKTVGRPAFKRFDIDSCTYYFSWDSRAACAVKPQEVQMVNGTITNPINGKSFSLGDIYFKLFRASGDMRTNGDNYLYEIQLSSITSSRNPACSGANICQVKPNDQHFSRKVGTSDKTKYYLQDGDLDVVFASSSKCGKDKTKSVSSTIFFHCDPLVEDGIPEFSHETADCQYLFSWYTSAVCPLGVGFDSENPGDDGQMHKGLSERSQAVGAVLSLLLVALTCCLLALLLYKKERRETVISKLTTCCRRSSNVSYKYSKVNKEEETDENETEWLMEEIQLPPPRQGKEGQENGHITTKSVKALSSLHGDDQDSEDEVLTIPEVKVHSGRGAGAESSHPVRNAQSNALQEREDDRVGLVRGEKARKGKSSSAQQKTVSSTKLVSFHDDSDEDLLHI

SEQ ID NO: 47 - protein, 561 aa, Homo sapiens
LGFSLPPHLLFRPRLDLQFLQRFLQILKVLFPSWSSQNALMFLTLLCLTLLGDFDQFTCNLLYVSWRKDLTEHLHRLYFRGRAYYTLNVLRDDIDNPDQRISQDVERFCRQLSSMASKLIISPFTLVYYTYQCFQSTGWLGPVSIFGYFILGTVVNKTLMGPIVMKLVHQEKLEGDFRFKHMQIRVNAEPAAFYRAGHVEHMRTDRRLQRLLQTQRELMSKELWLYIGINTFDYLGSILSYVVIAIPIFSGVYGDLSPAELSTLVSKNAFVCIYLISCFTQLIDLSTTLSDVAGYTHRIGQLRETLLDMSLKSQDCEILGESEWGLDTPPGWPAAEPADTAFLLERVSISAPSSDKPLIKDLSLKISEGQSLLITGNTGTGKTSLLRVLGGLWTSTRGSVQMLTDFGPHGVLFLPQKPFFTDGTLREQVIYPLKEVYPDSGSADDERILRFLELAGLSNLVARTEGLDQQVDWNWYDVLSPGEMQRLSFARLFYLQPKYAVLDEATSALTEEVESELYRIGQQLGMTFISVGHRQSLEKFHSLVLKLCGGGRWELMRIKVE

SEQ ID NO: 48 - protein, 421 aa, Homo sapiens
MAATLILEPAGRCCWDEPVRIAVRGLAPEQPVTLRASLRDEKGALFQAHARYRADTLGELDLERAPALGGSFAGLEPMGLLWALEPEKPLVRLVKRDVRTPLAVELEVLDGHDPDPGRLLCRVRHERYFLPPGVRREPVRAGRVRGTLFLPPEPGPFPGIVDMFGTGGGLLEYRASLLAGKGFAVMALAYYNYEDLPKTMETLHLEYFEEAVNYLLSHPEVKGPGVGLLGISKGGELCLSMASFLKGITAAVVINGSVANVGGTLRYKGETLPPVGVNRNRIKVTKDGYADIVDVLNSPLEGPDQKSFIPVERAESTFLFLVGQDDHNWKSEFYANEACKRLQAHGRRKPQIICYPETGHYIEPPYFPLCRASLHALVGSPIIWGGEPRAHAMAQVDAWKQLQTFFHKHLGGHEGTIPSKV

SEQ ID NO: 49 - protein, 785 aa, Homo sapiens
  VARIANT (196)...(198): Xaa = Any Amino Acid
MPKAPKQQPPEPEWIGDGESTSPSGEAGRQGRNEQRGKREETARFFEELAVEDKQAGEEEKVLKEKEQQQQQQQQQQQKKKRDTRKGRRKKDVDDDGEEKELMERLKKLSVPTSDEEDEVPAPKPRGGKKTKGGNVFAALIQDQSEEEEEEEKHPPKPAKPEKNRINKAVSEEQQPALKGKKGKEEKSKGKAKVRXXXFFLPSQMEYERQVASLKAANAAENDFSVSQAEMSSRQAMLENASDIKLEKFSISAHGKELFVNADLYIVAGRRYGLVGPNGKGKTTLLKHIANRALSIPPNIDVLLCEQEVVADETPAVQAVLRADTKRLKLLEEERRLQGQLEQGDDTAAERLEKVYEELRATGAAAAEAKARRILAGLGFDPEMQNRPTQKFSGGWRMRVSLARALFMEPTLLMLDEPTNHLDLNAVIWLNNYLQGWRKTLLIVSHDQGFLDDVCTDIIHLDAQRLHYYRGNYMTFKKMYQQKQKELLKQYEKQEKKLKELKAGGKSTKQAEKQTKEALTRKQQKCRRKNQDEESQEAPELLKRPKEYTVRFTFPDPPPLSPPVLGLHGVTFGYQGQKPLFKNLDFGIDMDSRICIVGPNGVGKSTLLLLLTGKLTPTHGEMRKNHRLKIGFFNQQYAEQLRMEETPTEYLQRGFNLPYQDARKCLGRFGLESHAHTIQICKLSGGQKARVVFAELACREPDVLILDEPTNNLDIESIDALGEAINEYKGAVIVVSHDARLITETNCQLWVVEEQSVSQIDGDFEDYKREVLEALGEVMVSRPRE

SEQ ID NO: 50 - protein, 405 aa, Homo sapiens
KMLSSFLSPQNGTWADTFSLLLALAVALYLGYYWACVLQRPRLVAGPQFLAFLEPHCSITTETFYPTLWCFEGRLQSIFQVLLQSQPLVLYQSDILQTPDGGQLLLDWAKQPDSSQDPDPTTQPIVLLLPGITGSSQDTYVLHLVNQALRDGYQAVVFNNRGCRGEELRTHRAFCASNTEDLETVVNHIKHRYPQAPLLAVGISFGGILVLNHLAQARQAAGLVAALTLSACWDSFETTRSLETPLNSLLFNQPLTAGLCQLVERNRKVIEKVVDIDFVLQARTIRQFDERYTSVAFGYQDCVTYYKAASPRTKIDAIRIPVLYLSAADDPFSPVCALPIQAAQHSPYVALLITARGGHIGFLEGLLPWQHWYMSRLLHQYAKAIFQDPEGLPDLRALLPSEDRN

SEQ ID NO: 51 - skipped

SEQ ID NO: 52 - protein, 284 aa, Homo sapiens
LFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTWLLHIFIPFCIIAFIFIATVIALRKQLCQKLYSSKDVSIHCAKVTLLVPIPTQTTVLQDYSSYGSPTHALSLVPKQDPYGLMR

SEQ ID NO: 53 - protein, 442 aa, Homo sapiens
MARGYGATVSLVLLGLGLALAVIVLAVVLSRHQAPCGPQAFAHAAVAADSKVCSDIGRAILQQQGSPVDATIAALVCTSVVNPQSMGLGGGVIFTIYNVTTGAQWIGVPGELRGYAEAHRRHGRLPWAQLFQPTIALLRGGHVVAPVLSRFLHNSILRPSLQASTLRQLFFNGTEPLRPQDPLPWPALATTLETVATEGVEVFYTGRLGQMLVEDIAKEGSQLTLQDLAKFQPEVVDALEVPLGDYTLYSPPPPAGGAILSFILNVLRGFNFSTESMARPEGRVNVYHHLVETLKFAKGQRWRLGDPRSHPKLQNASRDLLGETLAQLIRQQIDGRGDHQLSHYSLAEAWGHGTGTSHVSVLGEDGSAVAATSTINTPFGAMVYSPRTGIILNNELLDLCERCPRGSGTTPSPVETGWVELPEGAGPQFQASVPHPPWCPPS

SEQ ID NO: 54 - protein, 1047 aa, Homo sapiens
MAVTLDKDAYYRRVKRLYSNWRKGEDEYANVDAIVVSVGVDEEIVYAKSTALQTWLFGYELTDTIMVFCDDKIIFMASKKKVEFLKQIANTKGNENANGAPAITLLIREKNESNKSSFDKMIEAIKESKNGKKIGVFSKDKFPGEFMKSWNDCLNKEGFDKIDISAVVAYTIAVKEDGELNLMKKAASITSEVFNKFFKERVMEIVDADEKVRHSKLAESVEKAIEEKKYLAGADPSTVEMCYPPIIQSGGNYNLKFSVVSDKNHMHFGAITCAMGIRFKSYCSNLVRTLMVDPSQEVQENYNFLLQLQEELLKELRHGVKICDVYNAVMDVVKKQKPELLNKITKNLGFGMGIEFREGSLVINSKNQYKLKKGMVFSINLGFSDLTNKEGKKPEEKTYALFIGDTVLVDEDGPATVLTSVKKKVKNVGIFLKNEDEEEEEEEKDEAEDLLGRGSRAALLTERTRNEMTAEEKRRAHQKELAAQLNEEAKRRLTEQKGEQQIQKARKSNVSYKNPSLMPKEPHIREMKIYIDKKYETVIMPVFGIATPFHIATIKNISMSVEGDYTYLRINFYCPGSALGRNEGNIFPNPEATFVKEITYRASNIKAPGEQTVPALNLQNAFRIIKEVQKRYKTREAEEKEKEGIVKQDSLVINLNRSNPKLKDLYIRPNIAQKRMQGSLEAHVNGFRFTSVRGDKVDILYNNIKHALFQPCDGEMIIVLHFHLKNAIMFGKKRHTDVQFYTEVGEITTDLGKHQHMHDRDDLYAEQMEREMRHKLKTAFKNFIEKVEALTKEELEFEVPFRDLGFNGAPYRSTCLLQPTSSALVNATEWPPFVVTLDEVELIHFERVQFHLKNFDMVIVYKDYSKKVTMINAIPVASLDPIKEWLNSCDLKYTEGVQSLNWTKIMKTIVDDPEGFFEQGGWSFLEPEGEGSDAEEGDSESEIEDETFNPSEDDYEEEEEDSDEDYSSEAEESDYSKESLGSEEESGKDWDELEEEARKADRESRYEEEEEQSRSMSRKRKASVHSSGRGSNRGSRHSSAPPKKKRK

SEQ ID NO: 55 - protein, 1530 aa, Homo sapiens
SPILCGAATALNCSLCPQDSNLSVHTENPDLTPCFQNSLLAWVPCIYLWVALPCYLLYLRHHCRGYIILSHLSKLKMVLGVLLWCVSWADLFYSFHGLVHGRAPAPVFFVTPLVVGVTMLLATLLIQYERLQGVQSSGVLIIFWFLCVVCAIVPFRSKILLAKAEGEISDPFRFTTFYIHFALVLSALILACFREKPPFFSAKNVDPNPYPETSAGFLSRLFFWWFTKMAIYGYRHPLEEKDLWSLKEEDRSQMVVQQLLEAWRKQEKQTARHKASAAPGKNASGEDEVLLGARPRPRKPSFLKALLATFGSSFLISACFKLIQDLLSFINPQLLSILIRFISNPMAPSWWGFLVAGLMFLCSMMQSLILQHYYHYIFVTGVKFRTGIMGVIYRKALVITNSVKRASTVGEIVNLMSVDAQRFMDLAPFLNLLWSAPLQIILAIYFLWQNLGPSVLAGVAFMVLLIPLNGAVAVKMRAFQVKQMKLKDSRIKLMSEILNGIKVLKLYAWEPSFLKQVEGIRQGELQLLRTAAYLHTTTTFTWMCSPFLVTLITLWVYVYVDPNNVLDAEKAFVSVSLFNILRLPLNMLPQLISNLTQASVSLKRIQQFLSQEELDPQSVERKTISPGYAITIHSGTFTWAQDLPPTLHSLDIQVPKGALVAVVGPVGCGKSSLVSALLGEMEKLEGKVHMKGSVAYVPQQAWIQNCTLQENVLFGKALNPKRYQQTLEACALLADLEMLPGGDQTEIGEKGINLSGGQRQRVSLARAVYSDADIFLLDDPLSAVDSHVAKHIFDHVIGPEGVLAGKTRVLVTHGISFLPQTDFIIVLADGQVSEMGPYPALLQRNGSFANFLCNYAPDEDQGHLEDSWTALEGAEDKEALLIEDTLSNHTDLTDNDPVTYVVQKQFMRQLSALSSDGEGQGRPVPRRHLGPSEKVQVTEAKADGALTQEEKAAIGTVELSVFWDYAKAVGLCTTLAICLLYVGQSAAAIGANVWLSAWTNDAMADSRQNNTSLRLGVYAALGILQGFLVMLAAMAMAAGGIQAARVLHQALLHNKIRSPQSFFDTTPSGRILNCFSKDIYVVDEVLAPVILMLLNSFFNAISTLVVIMASTPLFTVVILPLAVLYTLVQRFYAATSRQLKRLESVSRSPIYSHFSETVTGASVIRAYNRSRDFEIISDTKVDANQRSCYPYIISNRWLSIGVEFVGNCVVLFAALFAVIGRSSLNPGLVGLSVSYSLQVTFALNWMIRMMSDLESNIVAVERVKEYSKTETEAPWVVEGSRPPEGWPPRGEVEFRNYSVRYRPGLDLVLRDLSLHVHGGEKVGIVGRTGAGKSSMTLCLFRILEAAKGEIRIDGLNVADIGLHDLRSQLTIIPQDPILFSGTLRMNLDPFGSYSEEDIWWALELSHLHTFVSSQPAGLDFQCSEGGENLSVGQRQLVCLARALLRKSRILVLDEATAAIDLETDNLIQATIRTQFDTCTVLTIAHRLNTIMDYTRVLVLDKGVVAEFDSPANLIAARGIFYGMARDAGLA

SEQ ID NO: 56 - protein, 533 aa, Homo sapiens
PYCSLPRAPLHGFILGQTSTQPGGSIHFGCNAGYRLVGHSMAICTRHPQGYHLWSEAIPLCQALSCGLPEAPKNGMVFGKEYTVGTKAMYSCSEGYHLQAGAEATAECLDTGLWSNRNVPPQCVPVTCPDVSSISVEHGRWRLIFETQYQFQAQLMLICDPGYYYTGQRVIRCQANGKWSLGDSTPTCRILAKQKQPCPSSWGWLTEHLVIILVISCGELPIPPNGHRIGTLSVYGATAIFSCNSGYTLVGSRVRECMANGLWSGSEVRCLATQTKLHSIFYKLLFDVLSSPSLTKAGHCGTPEPIVNGHINGENYSYRGSVVYQCNAGFRLIGMSVRICQQDHHWSGKTPFCVPITCGHPGNPVNGLTQGNQFNLNDVVKFVCNPGYMAEGAARSQCLASGQWSDMLPTCRIINCTDPGHQENSVRQVHASGPHRFSFGTTVSYRCNHGFYLLGTPVLSCQGDGTWDRPRPQCLLVSCGHPGSPPHSQMSGDSYTVGAVVRYSCIGKRTLVGNSTRMCGLDGHWTGSLPHCS

SEQ ID NO: 57 - protein, 1584 aa, Homo sapiens
PLAFCGSENHSAAYRVDQGVLNNGCFVDALNVVPHVFLLFITFPILFIGWGSQSSKVHIHHSTWLHFPGHNLRWILTFMLLFVLVCEIAEGILSDGVTESHHLHLYMPAGMAFMAAVTSVVYYHNIETSNFPKLLIALLVYWTLAFITKTIKFVKFLDHAIGFSQLRFCLTGLLVILYGMLLLVEVNVIRVRRYIFFKTPREVKPPEDLQDLGVRFLQPFVNLLSKGTYWWMNAFIKTAHKKPIDLRAIGKLPIAMRALTNYQRLCEAFDAQVRKDIQGTQGARAIWQALSHAFGRRLVLSSTFRILADLLGFAGPLCIFGIVDHLGKENDVFQPKTQFLGVYFVSSQEFLANAYVLAVLLFLALLLQRTFLQASYYVAIETGINLRGAIQTKIYNKIMHLSTSNLSMGEMTAGQICNLVAIDTNQLMWFFFLCPNLWAMPVQIIVGVILLYYILGVSALIGAAVIILLAPVQYFVATKLSQAQRSTLEYSNERLKQTNEMLRGIKLLKLYAWENIFRTRVETTRRKEMTSLRAFAIYTSISIFMNTAIPIAAVLITFVGHVSFFKEADFSPSVAFASLSLFHILVTPLFLLSSVVRSTVKALVSVQKLSEFLSSAEIREEQCAPHEPTPQGPASKYQAVPLRVVNRKRPAREDCRGLTGPLQSLVPSADGDADNCCVQIMGGYFTWTPDGIPTLSNITIRIPRGQLTMIVGQVGCGKSSLLLAALGEMQKVSGAVFWSSMPFLPCCSPERETATDLDIRKRGPVAYASQKPWLLNATVEENIIFESPFNKQRYKMVIEACSLQPDIDILPHGDQTQIGERGINLSGGQRQRISVARALYQHANVVFLDDPFSALDIHLSDHLMQAGILELLRDDKRTVVLVTHKLQYLPHADWIIAMKDGTIQREGTLKDFQRSECQLFEHWKTLMNRQDQELEKETVTERKATEPPQGLSRAMSSRDGLLQDEEEEEEEAAESEEDDNLSSMLHQRAEIPWRACAKYLSSAGILLLSLLVFSQLLKHMVLVAIDYWLAKWTDSALTLTPAARNCSLSQECTLDQTVYAMVFTVLCSLGIVLCLVTSVTVEWTGLKVAKRLHRSLLNRIILAPMRFFETTPLGSILNRFSSDCNTIDQHIPSTLECLSRSTLLCVSALAVISYVTPVFLVALLPLAIVCYFIQKYFRVASRDLQQLDDTTQLPLLSHFAETVEGLTTIRAFRYEARFQQKLLEYTDSNNIASLFLTAANRWLEVRMATPLPPQEYIGACVVLIAAVTSISNSLHRELSAGLVGLGLTYALMVSNYLNWMVRNLADMELQLGAVKRIHGLLKTEAESYEGLLAPSLIPKNWPDQGKIQIQNLSVRYDSSLKPVLKHVNALIAPGQKIGICGRTGSGKSSFSLAFFRMVDTFEGHIIIDGIDIAKLPLHTLRSRLSIILQDPVLFSGTIRFNLDPERKCSDSTLWEALEIAQLKLVVKALPGGLDAIITEGGENFSQGQRQLFCLARAFVRKTSIFIMDEATASIDMATENILQKVVMTAFADRTVVTIAHRVHTILSADLVIVLKRGAILEFDKPEKLLSRKDSVFASFVRADK

SEQ ID NO: 58 - protein, 274 aa, Homo sapiens
MPRNLLYSLLSSHLSPHFSTSVTSAKVAVNGVQLHYQQTGEGDHAVLLLPGMLGSGETDFGPQLKNLNKKLFTVVAWDPRGYGHSRPPDRDFPADFFERDAKDAVDLMKALKFKKVSLLGWSDGGITALIAAAKYPSYIHKMVIWGANAYVTDEDSMIYEGIRDVSKWSERTRKPLEALYGYDYFARTCEKWVDGIRQFKHLPDGNICRHLLPRVQCPALIVHGEKDPLVPRFHADFIHKHVKGSRLHLMPEGKHNLHLRFADEFNKLAEDFLQ

SEQ ID NO: 59 - protein, 1247 aa, Homo sapiens
MMREWVLLMSVLLCGLAGPTHLFQPSLVLDMAKVLLDNYCFPENLLGMQEAIQQAIKSHEILSISDPQTLASVLTAGVQSSLNDPRLVISYEPSTPEPPPQVPALTSLSEEELLAWLQRGLRHEVLEGNVGYLRVDSVPGQEVLSMMGEFLVAHVWGNLMGTSALVLDLRHCTGGQVSGIPYIISYLHPGNTILHVDTIYNRPSNTTTEIWTLPQVLGERYGADKDVVVLTSSQTRGVAEDIAHILKQMRRAIVVGERTGGGALDLRKLRIGESDFFFTVPVSRSLGPLGGGSQTWEGSGVLPCVGTPAEQALEKALAILTLRSALPGVVHCLQEVLKDYYTLVDRVPTLLQHLASMDFSTVVSEEDLVTKLNAGLQAASEDPRLLVRAIGPTETPSWPAPDAAAEDSPGVAPELPEDEAIRQALVDSVFQVSVLPGNVGYLRFDSFADASVLGVLAPYVLRQVWEPLQDTEHLIMDLRHNPGGPSSAVPLLLSYFQGPEAGPVHLFTTYDRRTNITQEHFSHMELPGPRYSTQRGVYLLTSHRTATAAEEFAFLMQSLGWATLVGEITAGNLLHTRTVPLLDTPEGSLALTVPVLTFIDNHGEAWLGGGVVPDAIVLAEEALDKAQEVLEFHQSLGALVEGTGHLLEAHYARPEVVGQTSALLRAKLAQGAYRTAVDLESLASQLTADLQEVSGDHRLLVFHSPGELVVEEAPPPPPAVPSPEELTYLIEALFKTEVLPGQLGYLRFDAMAELETVKAVGPQLVRLVWQQLVDTAALVIDLRYNPGSYSTAIPLLCSYFFEAEPRQHLYSVFDRATSKVTEVWTLPQVAGQRYGSHKDLYILMSHTSGSAAEAFAHTMQDLQRATVIGEPTAGGALSVGIYQVGSSPLYASMPTQMAMSATTGKAWDLAGVEPDITVPMSEALSIAQDIVALRAKVPTVLQTAGKLVADNYASAELGAKMATKLSGLQSRYSRVTSEVALAEILGADLQMLSGDPHLKAAHIPENAKDRIPGIVPMQIPSPEVFEELIKFSFHTNVLEDNIGYLRFDMFGDGELLTQVSRLLVEHIWKKIMHTDAMIIDMRFNIGGPTSSIPILCSYFFDEGPPVLLDKIYSRPDDSVSELWTHAQVVGERYGSKKSMVILTSSVTAGTAEEFTYIMKRLGRALVIGEVTSGGCQPPQTYHVDDTNLYLTIPTARSVGASDGSSWEGVGVTPHVVVPAEEALARAKEMLQHNQLRVKRSPGLQDHL

SEQ ID NO: 60 - protein, 300 aa, Homo sapiens
MAEVNIIYVTVFILKGITNRPELQAPCFGVFLVIYLVTVLGNLGLITLIKIDTRLHTPMYYFLSHLAFVDLCYSSAITPKMMVNFVVERNTIPFHACATQLGCFLTFMITECFLLASMAYDCYVAICSPLHYSTLMSRRVCIQLVAVPYIYSFLVALFHTVITFRLTYCGPNLINHFYCDDLPFLALSCSDTHMKEILIFAFAGFDMISSSSIVLTSYIFIIAAILRIRSTQGQHKAISTCGSHMVTVTIFYGTLIFMYLQPKSNHSLDTDKMASVFYTVVIPMLNPLIYSLRNKEVKDA

SEQ ID NO: 61 - protein, 418 aa, Homo sapiens
MIQLTATPVSALVDEPVHIRATGLIPFQMVSFQASLEDENGDMFYSQAHYRANEFGEVDLNHASSLGGDYMGVHPMGLFWSLKPEKLLTRLLKRDVMNRPFQVQVKLYDLELIVNNKVASAPKASLTLERWYVAPGVTRIKVREGRLRGALFLPPGEGLFPGVIDLFGGLGGLLEFRASLLASRGFASLALAYHNYEDLPRKPEVTDLEYFEEAANFLLRHPKVFGSGVGVVSVCQGVQIGLSMAIYLKQVTATVLINGTNFPFGIPQVYHGQIHQPLPHSAQLISTNALGLLELYRTFETTQVGASQYLFPIEEAQGQFLFIVGEGDKTINSKAHAEQAIGQLKRHGKNNWTLLSYPGAGHLIEPPYSPLCCASTTHDLRLHWGGEVIPHAAAQEHAWKEIQRFLRKHLIPDVTSQL

SEQ ID NO: 62 - protein, 262 aa, Homo sapiens
ISPQSRDAKPNPEEPIDEDEDIQTERIRTATALTTSILDEVELKGCSSVLGHLGYCPQENVLWPMLTLREHLEVYAAVKGLRKADARLAIARLVSAFKLHEQLNVPVQKLTAGITRKLCFVLSLLGNSPVLLLDEPSTGIDPTGQQQMWQAIQAVVKNTERGVLLTTHNLAEAEALCDRVAIMVSGRLRCIGSIQHLKNKLGKDYILELKVKETSQVTLVHTEILKLFPQAAGQERYSSLLTYKLPVADVYPLSQTFHKLEA

SEQ ID NO: 63 - protein, 1303 aa, Homo sapiens
WNTSNPDFTKCFQNTVLVWVPCFYLWACFPFYFLYLSRHDRGYIQMTPLNKTKTALGFLLWIVCWADLFYSFWERSRGIFLAPVFLVSPTLLGITMLLATFLIQLERRKGVQSSGIMLTFWLVALVCALAILRSKIMTALKEVDLFRDITFYVYFSLLLIQLVLSCFSDRSPLFSETIHDPNPCPESSASFLSRITFWWITGLIVRGYRQPLEGSDLWSLNKEDTSEQVVPVLVKNWKKECAKTRNSSGSGESCSANTEALFPAPTCHKSFQALSLLLCRLLIKFVNDTKAPDWQGYFYTVLLFVTACLQTLVLHQYFHICFVSGMRIKTAVIGAVYRKALVITNSARKSSTVGEIVNLMSVDAQRFMDLATYINMIWSAPLQVILALYLLWLVVAPDVLTAVSSKVAHMKSKDNRIKLMNEILNGIKVLKLYAWELAFKDKVLAIRQEELKVLKKSAYLSAVGTFTWVCTPFLVALCTFAVYVTIDENNILDAQTAFVSLALFNILRFPLNILPMVISSIVQVQGEAGATSERGPWGSRPRKHGTRQASFSVAEPGVLCRFSITFSIPEGALVAVVGQVGCGKSSLLSALLAEMDKVEGHVAIKGSVAYVPQQAWIQNDSLRENILFGCQLEEPYYRSVIQACALLPDLEILPSGDRTEIGEKGVNLSGGQKQRVSLARAVYSNADIYLFDDPLSAVDAHVGKHIFENVIGPKGMLKNKSCLISCDLQVKLSVYWDYMKAIGLFISFLSIFLFMCNHVSALASNYWLSLWTDDPIVNGTQEHTKVRLSVYGALGISQGIAVFGYSMAVSIGGILASRCLHVDLLHSILRSPMSFFERTPSGNLVNRFSKELDTVDSMIPEVIKMFMGSLFNVIGACIVILLATPIAAIIIPPLGLIYFFVQRFYVASSRQLKRLESVSRSPVYSHFNETLLGVSVIRAFEEQERFIHQSDLKVDENQKAYYPSIVANRWLAVRLECVGNCIVLFAALFAVISRHSLSAGLVGLSVSYSLQVTTYLNWLVRMSSEMETNIVAVERLKEYSETEKEAPWQIQETAPPSSWPQVGRVEFRNYCLRYREDLDFVLRHINVTINGGEKVGIVGRTGAGKSSLTLGLFRINESAEGEIIIDGINIAKIGLHDLRFKITIIPQDPVLFSGSLRMNLDPFSQYSDEEVWTSLELAHLKDFVSALPDKLDHECAEGGENLSVGQRQLVCLARALLRKTKILVLDEATAAVDLETDDLIQSTIRTQFEDCTVLTIAHRLNTIMDYTRVIVLDKGEIQEYGAPSDLLQQRGLFYSMAKDAGLV

SEQ ID NO: 64 - protein, 736 aa, Homo sapiens
HRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPEILHGEHTPSHQDNFSPGQEVFYSCEPGYDLRGAASLHCTPQGDWSPEAPRCAVKSCDDFLGQLPHGRVLFPLNLQLGAKVSFVCDEGFRLKGSSVSHCVLVGMRSLWNNSVPVCEHIFCPNPPAILNGRHTGTPSGDIPYGKEISYTCDPHPDRGMTFNLIGESTIRCTSDPHGNGVWSSPAPRCELSVRAGHCKTPEQFPFASPTIPINDFEFPVGTSLNYECRPGYFGKMFSISCLENLVWSSVEDNCRRKSCGPPPEPFNGMVHINTDTQFGSTVNYSCNEGFRLIGSPSTTCLVSGNNVTWDKKAPICEIISCEPPPTISNGDFYSNNRTSFHNGTVVTYQCHTGPDGEQLFELVGERSIYCTSKDDQVGVWSSPPPRCISTNKCTAPEVENAIRVPGNRSFFTLTEIIRFRCQPGFVMVGSHTVQCQTNGRWGPKLPHCSRVCQPPHILHGEHTLSHQDNFSPGQEVFYSCEPSYDLRGAASLHCTPQGDWSPEAPRCTVKSCDDFLGQLPHGRVLLPLNLQLGAKVSFVCDEGFRLKGRSASHCVLAGMKALWNSSVPVCER

SEQ ID NO: 65 - protein, 738 aa, Homo sapiens
MRGPPAWPLRLLEPPSPAEPGRLLPVACVWAAASRVPGSLSPFTGLRPARLWGAGPALLWGVGAARRWRSGCRGGGPGASRGVLGLARLLGLWARGPGSCRCGAFAGPGAPRLPRARFPGGPAAAAWAGDEAWRRGPAAPPGDKGRLRPAAAGLPEARKLLGLAYPERRRLAAAVGFLTMSSVISMSAPFFLGKIIDVIYTNPTVDYSDNLTRLCLGLSAVFLCGAAANAIRVYLMQTSGQRIVNRLRTSLFSSILRQEVAFFDKTRTGELINRLSSDTALLGRSVTENLSDGLRAGAQASVGISMMFFVSPNLATFVLSVVPPVSIIAVIYGRYLRKLTKVTQDSLAQATQLAEERIGNVRTVRAFGKEMTEIEKYASKVDHVMQLARKEAFARAGFFGATGLSGNLIVLSVLYKGGLLMGSAHMTVGELSSFLMYAFWVGISIGGLSSFYSELMKGLGAGGRLWELLEREPKLPFNEGVILNEKSFQGALEFKNVHFAYPARPEVPIFQDFSLSIPSGSVTALVGPSGSGKSTVLSLLLRLYDPASGTISLDGHDIRQLNPVWLRSKIGTVSQEPILFSCSIAENIAYGADDPSSVTAEEIQRVAEVANAVAFIRNFPQGFNTVVGEKGVLLSGGQKQRIAIARALLKNPKILLLDEATSALDAENEYLVQEALDRLMDGRTVLVIAHRLSTIKNANMVAVLDQGKITEYGKHEELLSKPNGIYRKLMNKQSFISA

SEQ ID NO: 66 - protein, 1273 aa, Homo sapiens
GILTWYIEAVHPGMYGLPRPWYFPLQKSYWLGSGRTEAWEWSWPWARTPRLSVMEEDQACAMESRRFEETRGMEEEPTHLPLVVCVDKLTKVYKDDKKLALNKLSLNLYENQVVSFLGHNGAGKTTHHNVLFDRLTVEEHLWFYSRLKSMAQEEIRREMDKMIEDLELSNKRHSLVQTLSGGMKRKLSVAIAFVGGSRAIILDEPTAGVDPYARRAIWDLILKYKPGRTILLSTHHMDEADLLGDRIAIISHGKLKCCGSPLFLKGTYGDGYRLTLVKRPAEPGGPQEPGLASSPPGRAPLSSCSELQVSQFIRKHVASCLLVSDTSTELSYILPSEAAKKGAFERLFQHLERSLDALHLSSFGLMDTTLEEVFLKVSEEDQSLENSEADVKESRKDVLPGAEGPASGEGHAGNLARCSELTQSQASLQSASSVGSARGDEGAGYTDVYGDYRPLFDNPQDPDNVSLQEVEAEALSRVGQGSRKLDGGWLKVRQFHGLLVKRFHCARRNSKALFSQILLPAFFVCVAMTVALSVPEIGDLPPLVLSPSQYHNYTQPRGNFIPYANEERREYRRHPAGASLVGGASEGAGTALVGQAGEGAGLARGGDWQPHLTLIWGRGGEDLGPESAAPAPPCAGITVTNHPMNKTSASLSLDYLLQGTDVVIAIFIIVAMSFVPASFVVFLVAEKSTKAKHLQFVSGCNPIIYWLANYVWDMLNYLVPATCCVIILFVFDLPAYTSPTNFPAVLSLFLLYGWSITPIMYPASFWFEVPSSAYVFLIVINLFIGITATVATFLLQLFEHDKDLKVVNSYLKSCFLIFPNYNLGHGLMEMAYNEYINEYYAKIGQFDKMKSPFEWDIVTRGLVAMAVEGVVGFLLTIMCQYNFLRRPQRMPVSTKPVEDDVDVASERQRVLRGDSDNDMCFGLLGVNGAGKTSTFKMLTGDESTTGGEAFVNGHSVLKELLQVQQSLGYCPQCDALFDELTAREHLQLYTRLRGISWKDEARVVKWALEKLELTKYADKPAGTYSGGNKRKLSTAIALIGYPAFIFLDEPTTGMDPKARRFLWNLILDLIKTGRSVVLTSHSMEECEALCTRLAIMVNGRLRCLGSIQHLKNRFGDGYMITVRTKSSQSVKDVVRFFNRNFPEAMLKERHHTKVQYQLKSEHISLAQVFSKMEQVSGVLGIEDYSVSQTTLDNVFVNFAKKQSDNLEQQETEPPSALQSPLGCLLSLLRPRSAPTELRALVADEPEDLDTEDEGLISFEEERAQLSFNTDTLC

SEQ ID NO: 67 - protein, 1479 aa, Homo sapiens [sequence omitted]

SEQ ID NO: 68 - protein, 583 aa, Homo sapiens
MKYILVTGGVISGIGKGIIASSIGTILKSCGLRVTAIKIDPYINIDAGTFSPYEHGEVFVLNDGGEVDLDLGNYERFLDINLYKDNNITTGKIYQHVINKERRGDYLGKTVQVVPHITDAVQEWVMNQAKVPVDGNKEEPQICLGGTIGDIEGMPFVEAFRQFQFKAKRENFCNIHVSLVPQLSATGEQKTKPTQNSVRALRGLGLSPDLIVCRSSTPIEMAVKEKISMFCHVNPEQVICIHDVSSTYRVPVLLEEQSIVKYFKERLHLPIGDSASNLLFKWRNMADRYERLQKICSIALVGKYTKLRDCYASVFKALEHSALAINHKLNLMYIDSIDLEKITETEDPVKFHEAWQKLCKADGILVPGGFGIRGTLGKLQAISWARTKKIPFLGVCLGMQLAVIEFARNCLNLKDADSTEFRPNAPVPLVIDMPEHNPGNLGGTMRLGIRRTVFKTENSILRKLYGDVPFIEERHRHRFEVNPNLIKQFEQNDLSFVGQDVDGDRMEIIELANHPYFVGVQFHPEFSSRPMKPSPPYLGLLLAATGNLNAYLQQGCKLSSSDRYSDASDDSFSEPRIAELEIS

SEQ ID NO: 69 - protein, 1241 aa, Homo sapiens
SPILCGAATALNCSLCPQDSNLSVHTENPDLTPCFQNSLLAWVPCIYLWVALPCYLLYLRHHCRGYIILSHLSKLKMVLGVLLWCVSWADLFYSFHGLVHGRAPAPVFFVTPLVVGVTMLLATLLIQYERLQGVQSSGVLIIFWFLCVVCAIVPFRSKILLAKAEGEISDPFRFTTFYIHFALVLSALILACFREKPPFFSAKNVDPNPYPETSAGFLSRLFFWWFTKMAIYGYRHPLEEKDLWSLKEEDRSQMVVQQLLEAWRKQEKQTARHKASAAPGKNASGEDEVLLGARPRPRKPSFLKALLATFGSSFLISACFKLIQDLLSFINPQLLSILIRFISNPMAPSWWGFLVAGLMFLCSMMQSLILQHYYHYIFVTGVKFRTGIMGVIYRKALVITNSVKRASTVGEIVNLMSVDAQRFMDLAPFLNLLWSAPLQIILAIYFLWQNLGPSVLAGVAFMVLLIPLNGAVAVKMRAFQVKQMKLKDSRIKLMSEILNGIKVLKLYAWEPSFLKQVEGIRQGELQLLRTAAYLHTTTTFTWMCSPFLVTLITLWVYVYVDPNNVLDAEKAFVSVSLFNILRLPLNMLPQLISNLTQASVSLKRIQQFLSQEELDPQSVERKTISPGYAITIHSGTFTWAQDLPPTLHSLDIQVPKGALVAVVGPVGCGKSSLVSALLGEMEKLEGKVHMKGSVAYVPQQAWIQNCTLQENVLFGKALNPKRYQQTLEACALLADLEMLPGGDQTEIGEKGINLSGGQRQRVSLARAVYSDADIFLLDDPLSAVDSHVAKHIFDHVIGPEGVLAGKTRVLVTHGISFLPQTDFIIVLADGQVSEMGPYPALLQRNGSFANFLCNYAPDEDQGHLEDSWTALEGAEDKEALLIEDTLSNHTDLTDNDPVTYVVQKQFMRQLSALSSDGEGQGRPVPRRHLGPSEKVQVTEAKADGALTQEEKAAIGTVELSVFWDYAKAVGLCTTLAICLLYVGQSAAAIGANVWLSAWTNDAMADSRQNNTSLRLGVYAALGILQGFLVMLAAMAMAAGGIQAARVLHQALLHNKIRSPQSFFDTTPSGRILNCFSKDIYVVDEVLAPVILMLLNSFFNAISTLVVIMASTPLFTVVILPLAVLYTLVQRFYAATSRQLKRLESVSRSPIYSHFSETVTGASVIRAYNRSRDFEIISDTKVDANQRSCYPYIISNRSEAASLAPCSSRNSQQALWCSGSLSLLSPKQKTGPALPLPHFLLI

SEQ ID NO: 70 - protein, 573 aa, Homo sapiens
AFHQGSLILCLALQSDRLLIKGGRIINDDQSLYADVYLEDGLIKQIGENLIVPGGVKTIEANGRMVIPGGIDVNTYLQKPSQGMTAADDFFQGTRAALVGGTTMIIDHVVPEPGSSLLTSFEKWHEAADTKSCCDYSLHVDITSWYDGVREELEVLVQDKGVNSFQVYMAYKDVYQMSDSQLYEAFTFLKGLGAVILVHAENGDLIAQEQKRILEMGITGPEGHALSRPEELEAEAVFRAITIAGRINCPVYITKVMSKSAADIIALARKKGPLVFGEPIAASLGTDGTHYWSKNWAKAAAFVTSPPLSPDPTTPDYLTSLLACGDLQVTGSGHCPYSTAQKAVGKDNFTLIPEGVNGIEERMTVVWDKAVATGKMDENQFVAVTSTNAAKIFNLYPRKGRIAVGSDADVVIWDPDKLKTITAKSHKSAVEYNIFEGMECHGSPLVVISQGKIVFEDGNINVNKGMGRFIPRKAFPEHLYQRVKIRNKVFGLQGVSRGMYDGPVYEVPATPKYATPAPSAKSSPSKHQPPPIRNLHQSNFSLSGAQIDDNNPRRTGHRIVAPPGGRSNITSLG

SEQ ID NO: 71 - protein, 351 aa, Homo sapiens
MQRALPGARQHLGAILASASVVVKALCAAVLFLYLLSFAVDTGCLAVTPGYLFPPNFWIWTLATHGLMEQHVWDVAISLTTVVVAGRLLEPLWGALELLIFFSVVNVSVGLLGAFAYLLTYMASFNLVYLFTVRIHGALGFLGGVLVALKQTMGDCVVLRVPQVRVSVMPMLLLALLLLLRLATLLQSPALASYGFGLLSSWVYLRFYQRHSRGRGDMADHFAFATFFPEILQPVVGLLANLVHSLLVKVKICQKTVKRYDVGAPSSITISLPGTDPQDAERRRQLALKALNERLKRVEDQSIWPSMDDDEEESGAKVDSPLPSDKAPTPPGKGAAPESSLITFEAAPPTL

SEQ ID NO: 72 - protein, 745 aa, Homo sapiens [sequence omitted]

SEQ ID NO: 73 - protein, 334 aa, Homo sapiens
MDLDVVNMFVIAGGTLAIPILAFVASFLLWPSALIRIYYWYWRRTLGMQVRYVHHEDYQFCYSFRGRPGHKPSILMLHGFSAHKDMWLSVVKFLPKNLHLVCVDMPGHEGTTRSSLDDLSIDGQVKRIHQFVECLKLNKKPFHLVGTSMGGQVAGVYAAYYPSDVSSLCLVCPAGLQYSTDNQFVQRLKELQGSAAVEKIPLIPSTPEEMSEMLQLCSYVRFKVPQQILQGLVDVRIPHNNFYRKLFLEIVSEKSRYSLHQNMDKIKVPTQIIWGKQDQVLDVSGADMLAKSIANCQVELLENCGHSVVMERPRKTAKLIIDFLASVHNTDNNK

SEQ ID NO: 74 - protein, 559 aa, Homo sapiens
SDRLLIRGGRIVNDDQSFYADVHVEDGLIKQIGENLIVPGGIKTIDAHGLMVLPGGVDVHTRLQMPVLGMTPADDFCQGTKAALAGGTTMILDHVFPDTGVSLLAAYEQWRERADSAACCDYSLHVDITRWHESIKEELEALVKEKGVNSFLVFMAYKDRCQCSDSQMYEIFSIIRDLGALAQVHAENGDIVEEEQKRLLELGITGPEGHVLSHPEEVEAEAVYRAVTIAKQANCPLYVTKVMSKGAADAIAQAKRRGVVVFGEPITASLGTDGSHYWSKNWAKAAAFVTSPPVNPDPTTADHLTCLLSSGDLQVTGSAHCTFTTAQKAVGKDNFALIPEGTNGIEERMSMVWEKCVASGKMDENEFVAVTSTNAAKIFNFYPRKGRVAVGSDADLVIWNPKATKIISAKTHNLNVEYNIFEGVECRGAPAVVISQGRVALEDGKMFVTPGAGRFVPRKTFPDFVYKRIKARNRLAEIHGVPRGLYDGPVHEVMVPAKPGSGAPARASCPGKISVPPVRNLHQSGFSLSGSQADDHIARRTAQKIMAPPGGRSNITSLS

SEQ ID NO: 75 - protein, 743 aa, Homo sapiens
MARRSVLYFILLNALINKGQACFCDHYAWTQWTSCSKTCNSGTQSRHRQIVVDKYYQENFCEQICSKQETRECNWQRCPINCLLGDFGPWSDCDPCIEKQGTSNFHYLNHLFTSFFHLDSSFIRIHKVMKVLNFTTKAKDLHLSDVFLKALNHLPLEYNSALYSRIFDDFGTHYFTSGSLGGVYDLLYQFSSEELKNSGLTEEEAKHCVRIETKKRVLFAKKTKVEHRCTTNKLSEKHEGSFIQGAEKSISLIRGGRSEYGAALAWEKGSSGLEEKTFSEWLESVKENPAVIDFELAPIVDLVRNIPCAVTKRNNLRKALQEYAAKFDPCQCAPCPNNGRPTLSGTECLCVCQSGTYGENCEKQSPDYKSNAVDGQWGCWSSWSTCDATYKRSRTRECNNPAPQRGGKRCEGEKRQEEDCTFSIMENNGQPCINDDEEMKEVDLPEIEADSGCPQPVPPENGFIRNEKQLYLVGEDVEISCLTGFETVGYQYFRCLPDGTWRQGDVECQRTECIKPVVQEVLTITPFQRLYRIGESIELTCPKGFVVAGPSRYTCQGNSWTPPISNSLTCEKDTLTKLKGHCQLGQKQSGSECICMSPEEDCSHHSEDLCVFDTDSNDYFTSPACKFLAEKCLNNQQLHFLHIGSCQDGRQLEWGLERTRLSSNSTKKESCGYDTCYDWEKCSASTSKCVCLLPPQCFKGGNQLYCVKMGSSTSEKTLNICEVGTIRCANRKMEILHPGKCLA

SEQ ID NO: 76 - protein, 321 aa, Homo sapiens
MERKNQTAITEFIILGFSNLNELQFLLFTIFFLTYFCTLGGNILIILTTVTDPHLHTPMYYFLGNLAFIDICYTTSNVPQMMVHLLSKKKSISYVGCVVQLFAFVFFVGSECLLLAAMAYDRYIAICNPLRYSVILSKVLCNQLAASCWAAGFLNSVVHTVLTFCLPFCGNNQINYFFCDIPPLLILSCGNTSVNELALLSTGVFIGWTPFLCIVLSYICIISTILRIQSSEGRRKAFSTCASHLAIVFLFYGSAIFTYVRPISTYSLKKDRLVSVLYSVVTPMLNPIIYTLRNKDIKEAVKTIGSKWQPPISSLDSKLTY

SEQ ID NO: 77 - protein, 297 aa, Homo sapiens
MDPGKDKEGVPQPSGPPARKKFVIPLDEDEVPPGVAKPLFRSTQSLPTVDTSAQAAPQTYAEYAISQPLEGAGATCPTGSEPLAGETPNQALKPGAKSNSIIVSPRQRGNPVLKFVRNVPWEFGDVIPDYVLGQSTCALFLSLRYHNLHPDYIHGRLQSLGKNFALRVLLVQVDVKDPQQALKELAKMCILADCTLILAWSPEEAGRYLETYKAYEQKPADLLMEKLEQDFVSRVTECLTTVKSVNKTDSQTLLTTFGSLEQLIAASREDLALCPGLGPQKARRLFDVLHEPFLKVP

SEQ ID NO: 78 - protein, 579 aa, Homo sapiens
MLANSASVRILIKGGKVVNDDCTHEADVYIENGIIQQVGRELMIPGGAKVIDATGKLVIPGGIDTSTHFHQTFMNATCVDDFYHGTKAALVGGTTMIIGHVLPDKETSLVDAYEKCRGLADPKVCCDYALHVGITWWAPKVKAEMETLVREKGVNSFQMFMTYKDLNWNMLRDSELYQVLHACKDIGAIARVHAENGELVAEASLQPRILDGGTPGAKEALDLGITGPEGIEISRPEELEAEATHRVITIANRTHCPIYLVNVSSISAGDVIAAAKMQGKVVLAETTTAHATLTGLHYYHQDWSHAAAYVTVPPLRLDTNTSTYLMSLLANDTLNIVASDHRPFTTKQKAMGKEDFTKIPHGVSGVQDRMSVIWERGVVGGKMDENRFVAVTSSNAAKLLNLYPRKGRIIPGADADVVVWDPEATKTISASTQVQGGDFNLYENMRCHGVPLVTISRGRVVYENGVFMCAEGTGKFCPLRSFPDTVYKKLVQREKTLKVRGVDRTPYLGDVAVVVHPGKKEMGTPLADTPTRPVTRHGGMRDLHESSFSLSGSQIDDHVPKRASARILAPPGGRSSGIW

SEQ ID NO: 79 - protein, 366 aa, Homo sapiens
VTAVAQQNQGEVPEPQDMKVAEVLFDAADANAIEEVNLAYENVKEVDGLDVSKEGTEAWEAAMKRYDERIDRVETRITARLRDQLGTAKNANEMFRIFSRFNALFVRPHIRGAIREYQTQLIQRVKDDIESLHDKFKVQYPQSQACKMSHVRDLPPVSGSIIWAKQIDRQLTAYMKRVEDVLGKGWENHVEGQKLKQDGDSFRMKLNTQEIFDDWARKVQQRNLGVSGRIFTIESTRVRGRTGNVLKLKVNFLPEIITLSKEVRNLKWLGFRVPLAIVNKAHQANQLYPFAISLIESVRTYERTCEKVEERNTISLLVAGLKKEVQALIAEGIALVWESYKLDPYVQRLAETVFNFQEKVCSHVIL

SEQ ID NO: 80 - protein, 1335 aa, Homo sapiens
MSDSVILRSIKKFGEENDGFESDKSWWSLNPYVFLIRLQDEKKGDGVRVGFFQLFRFSSSTDIWLMFVGSLCAFLHGIAQPGVLLIFGTMTDVFIDYDVELQELQIPGKACVNNTIVWTNSSLNQNMTNGTRCGLLNIESEMIKFASYYAGIAVAVLITGYIQICFWVIAAARQIQKMRKFYFRRIMRMEIGWFDCNSVGELNTRFSDDINKINDAIADQMALFIQRMTSTICGFLLGFFRGWKLTLVIISVSPLIGIGAATIGLSVSKFTDYELKAYAKAGVVADEVISSMRTVAAFGGEKREVERYEKNLVFAQRWGIRKGIVMGFFTGFVWCLIFLCYALAFWYGSTLVLDEGEYTPGTLVQIFLSVIVGALNLGNASPCLEAFATGRAAATSIFETIDRKPIIDCMSEDGYKLDRIKGEIEFHNVTFHYPSRPEVKILNDLNMVIKPGEMTALVGPSGAGKSTALQLIQRFYDPCEGMVTVDGHDIRSLNIQWLRDQIGIVEQEPVLFSTTIAENIRYGREDATMEDIVQAAKEANAYNFIMDLPQQFDTLVGEGGGQMSGGQKQRVAIARALIRNPKILLLDMATSALDNESEAMVQEVLSKIQHGHTIISVAHRLSTVRAADTIIGFEHGTAVERGTHEELLERKGVYFTLVTLQSQGNQALNEEDIKGKCFFPILVLDATEDDMLARTFSRGSYQDSLRASIRQRSKSQLSYLVHEPPLAVVDHKSTYEEDRKDKDIPVQEEVEPAPVRRILKFSAPEWPYMLVGSVGAAVNGTVTPLYAFLFSQILGTFSIPDKEEQRSQINGVCLLFVAMGCVSLFTQFLQGYAFAKSGELLTKRLRKFGFRAMLGQDIAWFDDLRNSPGALTTRLATDASQVQGAAGSQIGMIVNSFTNVTVAMIIAFSFSWKLSLVILCFFPFLALSGATQTRMLTGFASRDKQALEMVGQITNEALSNIRTVAGIGKERRFIEALETELEKPFKTAIQKANIYGFCFAFAQCIMFIANSASYRYGGYLISNEGLHFSYVFRVISAVVLSATALGRAFSYTPSYAKAKISAARFFQLLDRQPPISVYNTAGEKWDNFQGKIDFVDCKFTYPSRPDSQVLNGLSVSISPGQTLAFVGSSGCGKSTSIQLLERFYDPDQGKVMIDGHDSKKVNVQFLRSNIGIVSQEPVLFACSIMDNIKYGDNTKEIPMERVIAAAKQAQLHDFVMSLPEKYETNVGSQGSQLSRGEKQRIAIARAIVRDPKILLLDEATSALDTESEKTVQVALDKAREGRTCIVIAHRLSTIQNADIIAVMAQGVVIEKGTHEELMAQKGAYYKLVTTGSPIS

SEQ ID NO: 81 - protein, 1279 aa, Homo sapiens
MDLEAAKNGTAWRPTSAEGDFELGISSKQKRKKTKTVKMIGVLTLFRYSDWQDKLFMSLGTIMAIAHGSGLPLMMIVFGEMTDKFVDTAGNFSFPVNFSLSLLNPGKILEEEMTRYAYYYSGLGAGVLVAAYIQVSFWTLAAGRQIRKIRQKFFHAILRQEIGWFDINDTTELNTRLTDDISKISEGIGDKVGMFFQAVATFFAGFIVGFIRGWKLTLVIMAISPILGLSAAVWAKILSAFSDKELAAYAKAGAVAEEALGAIRTVIAFGGQNKELERYQKHLENAKEIGIKKAISANISMGIAFLLIYASYALAFWYGSTLVISKEYTIGNAMTVFFSILIGAFSVGQAAPCIDAFANARGAAYVIFDIIDNNPKIDSFSERGHKPDSIKGNLEFNDVHFSYPSRANVKILKGLNLKVQSGQTVALVGSSGCGKSTTVQLIQRLYDPDEGTINIDGQDIRNFNVNYLREIIGVVSQEPVLFSTTIAENICYGRGNVTMDEIKKAVKEANAYEFIMKLPQKFDTLVGERGAQLSGGQKQRIAIARALVRNPKILLLDEATSALDTESEAEVQAALDKAREGRTTIVIAHRLSTVRNADVIAGFEDGVIVEQGSHSELMKKEGVYFKLVNMQTSGSQIQSEEFELNDEKAATRMAPNGWKSRLFRHSTQKNLKNSQMCQKSLDVETDGLEANVPPVSFLKVLKLNKTEWPYFVVGTVCAIANGGLQPAFSVIFSEIIAIFGPGDDAVKQQKCNIFSLIFLFLGIISFFTFFLQGFTFGKAGEILTRRLRSMAFKAMLRQDMSWFDDHKNSTGALSTRLATDAAQVQGATGTRLALIAQNIANLGTGIIISFIYGWQLTLLLLAVVPIIAVSGIVEMKLLAGNAKRDKKELEAAGKIATEAIENIRTVVSLTQERKFESMYVEKLYGPYRNSVQKAHIYGITFSISQAFMYFSYAGCFRFGAYLIVNGHMRFRDVILVFSAIVFGAVALGHASSFAPDYAKAKLSAAHLFMLFERQPLIDSYSEEGLKPDKFEGNITFNEVVFNYPTRANVPVLQGLSLEVKKGQTLALVGSSGCGKSTVVQLLERFYDPLAGTVLLDGQEAKKLNVQWLRAQLGIVSQEPILFDCSIAENIAYGDNSRVVSQDEIVSAAKAANIHPFIETLPHKYETRVGDKGTQLSGGQKQRIAIARALIRQPQILLLDEATSALDTESEKVVQEALDKAREGRTCIVIAHRLSTIQNADLIVVFQNGRVKEHGTHQQLLAQKGIYFSMVSVQAGTQNL

SEQ ID NO: 82 - protein, 1280 aa, Homo sapiens
MDLEGDRNGGAKKKNFFKLNNKSEKDKKEKKPTVSVFSMFRYSNWLDKLYMVVGTLAAIIHGAGLPLMMLVFGEMTDIFANAGNLEDLMSNITNRSDINDTGFFMNLEEDMTRYAYYYSGIGAGVLVAAYIQVSFWCLAAGRQIHKIRKQFFHAIMRQEIGWFDVHDVGELNTRLTDDVSKINEGIGDKIGMFFQSMATFFTGFIVGFTRGWKLTLVILAISPVLGLSAAVWAKILSSFTDKELLAYAKAGAVAEEVLAAIRTVIAFGGQKKELERYNKNLEEAKRIGIKKAITANISIGAAFLLIYASYALAFWYGTTLVLSGEYSIGQVLTVFFSVLIGAFSVGQASPSIEAFANARGAAYEIFKIIDNKPSIDSYSKSGHKPDNIKGNLEFRNVHFSYPSRKEVKILKGLNLKVQSGQTVALVGNSGCGKSTTVQLMQRLYDPTEGMVSVDGQDIRTINVRFLREIIGVVSQEPVLFATTIAENIRYGRENVTMDEIEKAVKEANAYDFIMKLPHKFDTLVGERGAQLSGGQKQRIAIARALVRNPKILLLDEATSALDTESEAVVQVALDKARKGRTTIVIAHRLSTVRNADVIAGFDDGVIVEKGNHDELMKEKGIYFKLVTMQTAGNEVELENAADESKSEIDALEMSSNDSRSSLIRKRSTRRSVRGSQAQDRKLSTKEALDESIPPVSFWRIMKLNLTEWPYFVVGVFCAIINGGLQPAFAIIFSKIIGVFTRIDDPETKRQNSNLFSLLFLALGIISFITFFLQGFTFGKAGEILTKRLRYMVFRSMLRQDVSWFDDPKNTTGALTTRLANDAAQVKGAIGSRLAVITQNIANLGTGIIISFIYGWQLTLLLLAIVPIIAIAGVVEMKMLSGQALKDKKELEGSGKIATEAIENFRTVVSLTQEQKFEHMYAQSLQVPYRNSLRKAHIFGITFSFTQAMMYFSYAGCFRFGAYLVAHKLMSFEDVLLVFSAVVFGAMAVGQVSSFAPDYAKAKISAAHIIMIIEKTPLIDSYSTEGLMPNTLEGNVTFGEVVFNYPTRPDIPVLQGLSLEVKKGQTLALVGSSGCGKSTVVQLLERFYDPLAGKVLLDGKEIKRLNVQWLRAHLGIVSQEPILFDCSIAENIAYGDNSRVVSQEEIVRAAKEANIHAFIESLPNKYSTKVGDKGTQLSGGQKQRIAIARALVRQPHILLLDEATSALDTESEKVVQEALDKAREGRTCIVIAHRLSTIQNADLIVVFQNGRVKEHGTHQQLLAQKGIYFSMVSVQAGTKRQ

SEQ ID NO: 83 - protein, 378 aa, Homo sapiens
MLLTVYCVRRDLSEVTFSLQVDADFELHNFRALCELESGIPAAESQIVYAERPLTDNHRSLASYGLKDGDVVILRQKENADPRPPVQFPNLPRIDFSSIAVPGTSSPRQRQPPGTQQSHSSPGEITSSPQGLDNPALLRDMLLANPHELSLLKERNPPLAEALLSGDLEKFSRVLVEQQQDRARREQERIRLFSADPFDLEAQAKIEEDIRQQNIEENMTIAMEEAPESFGQVVMLYINCKVNGHPVKAFVDSGAQMTIMSQACAERCNIMRLVDRRWAGIAKGVGTQKIIGRVHLAQVQIEGDFLPCSFSILEEQPMDMLLGLDMLKRHQCSIDLKKNVLVIGTTGSQTTFLPEGELPECARLAYGAGREDVRPEEI

SEQ ID NO: 84 - protein, 78 aa, Homo sapiens
QTGPSVTVTCTEGKWNKQCRIKCEDTAPHAVLPSGSECATSCLDHNSESIILPMNVTVRDIPHWLNPTRVEVSDQGHL

SEQ ID NO: 85 - protein, 417 aa, Homo sapiens
MSATLILEPPGRCCWNEPVRIAVRGLAPEQRVTLRASLRDEKGALFRAHARYCADARGELDLERAPALGGSFAGLEPMGLLWALEPEKPFWRFLKRDVQIPFVVELEVLDGHDPEPGRLLCQAQHERHFLPPGVRRQSVRAGRVRATLFLPPGPGPFPGIIDIFGIGGGLLEYRASLLAGHGFATLALAYYNFEDLPNNMDNISLEYFEEAVCYMLQHPQVKGPGIGLLGISLGADICLSMASFLKNVSATVSINGSGISGNTAINYKHSSIPPLGYDLRRIKVAFSGLVDIVDIRNALVGGYKNPSMIPIEKAQGPILLIVGQDDHNWRSELYAQTVSERLQAHGKEKPQIICYPGTGHYIEPPYFPLCPASLHRLLNKHVIWGGEPRAHSKAQEDAWKQILAFFCKHLGGTQKTA

SEQ ID NO: 86 - protein, 1066 aa, Homo sapiens
  VARIANT (116)...(117): Xaa = Any Amino Acid
ILHGEHTLSHQDNFSPGQEVFYSCEPSYDLRGAASLHCTPQGDWSPEAPRCTVKSCDDFLGQLPHGRVLLPLNLQLGAKVSFVCDEGFRLKGRSASHCVLAGMKALWNSSVPVCEXXMIKTVFLFFSLPISNNAHENPKEVAIHLHSQGGSSVHPRTLQTNEENSRYIHTEFKMFSTTQISKMETGLEYDIALANNECKNSYSLVTREIFVIHYIDCALPFPGIICGLPPTIANGDFTSISREYFHYGSVVTYHCNLGSRGKKVFELVGEPSIYCTSKDDQVGIWSGPAPQCIIPNKCTPPNVENGILVSDNRSLFSLNEVVEFRCQPGFGMKGPSHVKCQALNKWEPELPSCSRVCQPPPDVLHAERTQRDKDNFSPGQEVFYSCEPGYDLRGSTYLHCTPQGDWSPAAPRCEVKSCDDFLGQLPNGHVLFPLNLQLGAKVDFVCDEGFQLKGSSASYCVLAGMESLWNSSVPVCERVTFQANLSPSSVQYLTHDTLRTEESSDYSTWLQNIFFPTGKSCETPPVPVNGMVHVITDIHVGSRINYSCTTGHRLIGHSSAECILSGNTAHWSMKPPICQRIPCGLPPNITNGYFISTDREYFHYGSVVTYHCNLGSRGRKVFELVGEPSIYCTSKDDQVVVWSGPVPQCIIPNKCTPPNVENGILVSDNRSLFSLNEVVEFRCQPGFVMKGPHRVQCQALNKWEPELPSCSRGYSKRNSPITNKYSGTVLSTMCQPPPEILHGEHTLSHQDNFLPGQEVFYSCEPSYDLRGAASLHCMPQGDWTPEAPRCTGASLSPSHGSLTPVVLFFLLVKSCDDFLGQLPHGRVLFPLNLQLGAKVSFVCDEGSASHCVLAGTKALWNSSVPVCEQIFCPNPPAILNGRHTGTPPGDIPYGKEVSYTCDPHPDRGMTFNLIGESTIRRTSEPHGNGVWSSPAPRCELPVGADQCNVPEWLPFARPTNLTDDFEFPIGTYLNYECRPGYSGRPFSIICLKNSVWTSAKDKCKRKSCRNPPDPVNGMAHVIKDIQFRSQIKYSCPKGYRLIGSSSATCIISGNTVIWDNKTPVCD

SEQ ID NO: 87 - protein, 683 aa, Homo sapiens
ICCPDDPQPAKDQLATVPKDIPLDCDCVLTGEDILGEVANRTAQGLEGLVSDSACTVGTIDAEQLSDTDSVQMFLELEKECLCEEGVTPLVELQNQISSEGLAASQDAENLLVISHFSGAALEKEQHLGLLHVRAKDYDTRLDCGYFNTLDSSQVPNAVELIAHVDIMRDTSTVSKEECEKVPFSPRTAEFKSRQPADLDSLEKLDPGGLLNSDHRVSHEEKLSGFIASELAKDNGSLSQGDCSQTEGNGEECIERVTFSFAFNHELTDVTSGPEVEVLYESNLLTDEIHLESGNVTVNQENNSLTSMGNVVTCELSVEKVCDEDGEAKELDYQATLLEDQAPAHFHRNFPEQVFQDLQRKSPESEILSLHLLVEELRLNPDGVETVNDTKPELNVASSEGGEMERRDSDSFLNIFPEKQVTKAGNTEPVLEEWIPVLQRPSRTAAVPTVKDALDAALPSPEEGTSIAAVPAPEGTAVVAALVPFPHEDILVASIVSLEEEDVTAAAVSAPERATVPAVTVSVPEGTAAVAAVSSPEETAPAVAAAITQEGMSAVAGFSPEWAALAVTVPITEEDAAAVPTPEVAAIPAASVPTPEVPAIPAAAVPPMEEVSPIGVPFLGVSAHTDSVPISEEGTPVLEEASSTGMWIKEDLDSLVFGIKEVTSTVLHGKVPLAATAGLNSDE

SEQ ID NO: 88 - protein, 174 aa, Homo sapiens
SEGNKRRLSTAIALMGRSSVIFLDEPSTGMDPVARRLLWNMVTKTRESGKAIVMTSHSMEECDALCTSLAIMVQGKFTCLGSPQHLKSKFGNIYILKVKVKTEDKLEDFKCYVATTFPGEIAMVTVFLLLLLKVFGILEEAKEQFDLEDYSVSQITLEQVFLTFANPEKASSDD

SEQ ID NO: 89 - protein, 124 aa, Homo sapiens
DCGPPPELPFAFPINPLYDTEFKTGTTLKYTCHPGHGKINSSRLICDAKDSWNYSIFCAIAKCEPPPDIRNGKHSGGDQEFYTYASSVTYSCNPYFSLIGNVSISCTVENETIGVWSPNPPICE

SEQ ID NO: 90 - protein, 294 aa, Homo sapiens
ISKDRKERVHQGMVRAATVGYGILREGGSAVDAVEGAVVALEDDPEFNAGCGSVLNTNGEVEMDASIMDGKDLSAGAVSAVQCIANPIKLARLVMEKTPHCFLTDQGAAQFAAAMGVPEIPGEKLVTERNKKRLEKEKHEKGAQKTDCQKNLGTVGAVALDCKGNVAYATSTGGIVNKMVGRVGDSPCLGAGGYADNDIGAVSTTGHGESILKVNLARLTLFHIEQGKTVEEAADLSLGYMKSRVKGLGGLIVVSKTGDWVAKWTSTSMPWAAAKDGKLHFGIDPDDTTITDLP

SEQ ID NO: 91 - protein, 467 aa, Homo sapiens
LQLGAKVSFVCDEGFRLKGRSASHCVLAGMKALWNSSVPVCERIICGLPPTIANGDFTSISREYFHYGSVVTYHCNLGSRGKKVFELVGEPSIYCTSKDDQVGIWSGPAPQCIIPNKCTPPNVENGILVSDNRSLFSLNEVVEFRCQPGFGMKGPSHVKCQALNKWEPELPSCSRVCQPPPDVLHAERTQRDKDNFSPGQEVFYSCEPGYDLRGSTYLHCTPQGDWSPAAPRCEVKSCDDFLGQLPNGHVLFPLNLQLGAKVDFVCDEGFQLKGSSASYCVLAGMESLWNSSVPVCEQIFCPNPPAILNGRHTGTPPGDIPYGKEVSYTCDPHPDRGMTFNLIGESTIRRTSEPHGNGVWSSPAPRCELPVGADQCNVPEWLPFARPTNLTDDFEFPIGTYLNYECRPGYSGRPFSIICLKNSVWTSAKDKCKRKSCRNPPDPVNGMAHVIKDIQFRSQIKYSCPKG

SEQ ID NO: 92 - protein, 446 aa, Homo sapiens
HGRVLLPLNLQLGAKVSFVCDEGFRLKGRSASHCVLAGMKALWNSSVPVCERIICGLPPTIANGDFTSISREYFHYGSVVTYHCNLGSRGKKVFELVGEPSIYCTSKDDQVGIWSGPAPQCIIPNKCTPPNVENGILVSDNRSLFSLNEVVEFRCQPGFGMKGPSHVKCQALNKWEPELPSCSRVCQPPPDVLHAERTQRDKDNFSPGQEVFYSCEPGYDLRGSTYLHCTPQGDWSPAAPRCEVKSCDDFLGQLPNGHVLFPLNLQLGAKVDFVCDEGFQLKGSSASYCVLAGMESLWNSSVPVCEQIFCPNPPAILNGRHTGTPPGDIPYGKEVSYTCDPHPDRGMTFNLIGESTIRRTSEPHGNGVWSSPAPRCELPVGAGQYPLPHILNGFRICSEVEVFEYLNAVTDSCDPAPGPDPFSLIGESTIYCGDNSVWNHAAPECK